(19) United States
(12) Patent Application Publication
(10) Pub. No.: US 2010/0135910 A1
(43) Pub. Date: Jun. 3, 2010

(54) Modulation of Anergy and Methods for Isolating Anergy-Modulating Compounds

(75) Inventors: Anjana Rao, Cambridge, MA (US); Patrick G. Hogan, Cambridge, MA (US); Vigo Heissmeyer, Brookline, MA (US)

Correspondence Address: FISH & RICHARDSON PC, P.O. BOX 1022, MINNEAPOLIS, MN, 55440-1022, US

(73) Assignee: The CBR Institute for Biomedical Research, Inc., a Massachusetts corporation

(21) Appl. No.: 12/477,622

(22) Filed: Jun. 3, 2009

Related U.S. Application Data
(62) Division of application No. 10/575,932, filed on Dec. 26, 2006.
(60) Provisional application No. 60/512,235, filed on Oct. 17, 2003.

(51) Int. Cl. A61K 49/00; G01N 33/53; A61P 37/02

(52) U.S. Cl. 424/9.2; 435/7.1; 435/7.24

(57) ABSTRACT

The present invention provides methods for identifying compounds capable of modulating anergy by inhibiting the production or activity of anergy associated E3 ubiquitin ligases or by altering the interaction between a ligase and its substrate.

### STATEMENT AS TO FEDERALLY SPONSORED RESEARCH

This invention was made with Government support under National Institutes of Health Grant Nos. RO1AI48213, RO1AI40127, and RO3HD39685. The Government has certain rights in this invention.

### TECHNICAL FIELD

This invention relates to anergy-associated proteins and modulation of anergy.

### BACKGROUND

One of the salient features of the normal immune system is its ability to mount responses against foreign antigens while not attacking self-antigens. This discrimination is imposed largely during development in the thymus where many autoreactive T cells are triggered to undergo apoptosis in a process known as clonal deletion. However, there is at least a second mechanism for inducing tolerance outside the thymus in the periphery. This mechanism, also termed peripheral tolerance, can be induced by activation of T cell receptors (TCR) without costimulation.

Costimulation is necessary for a productive response to antigen (reviewed in Jenkins M. K., (1994) Immunity 1:443-446; Lenschow et al., (1996) Annu Rev Immunol 14:233-258; and Parijs et al. (1996) Science 280:243-248). In T cells, a predominant costimulatory receptor is CD28, which binds the costimulatory ligands B7-1 (CD80) and B7-2 (CD86) expressed on the surface of antigen-presenting cells (APC). Combined engagement of TCR and CD28 results in full activation of a number of signaling pathways that ultimately lead to IL-2 production and cell proliferation.

TCR engagement in the absence of costimulation results in a partial response. The incompletely stimulated T cells enter a long-lived unresponsive state, known as tolerance or anergy. Critically, once tolerance is induced, the anergic T cell is blocked from the response evoked by exposure to an antigen presented by an APC. In such cells, the combined engagement of the T cell receptor (TCR) and CD28 does not trigger the level of IL-2 production and the extent of proliferation that occurs in fully activated T cells (reviewed in Schwartz R. H., (1990) Science 248: 1349-1356, and Schwartz R. H., (1996) J Exp Med. 184(1):1-8).

Antigen binding to the B cell antigen receptor causes analogous biochemical and biological effects to antigen binding to the T cell receptor. B cell receptor ligation results in B cell proliferation and induces the expression of T cell costimulatory molecules such as B7-2, priming the B cell to produce antibodies. B cell receptor activation in the absence of CD19 costimulation results in a partial, tolerant or anergic response.

There is considerable evidence that tumors can induce immune tolerance in order to functionally inactivate T cells that may mount a tumor-specific response.

### SUMMARY

The present invention is based, in part, on the discovery that Ca²⁺-induced anergy is a multi-step program implemented, at least partly, through proteolytic degradation of specific signaling proteins. Without intending to be bound by theory, it is believed that calcineurin increases mRNA and protein levels of certain anergy-associated E3 ubiquitin ligases, such as Itch, Cbl-b and Grail, and induces expression of Tsg101, which is the ubiquitin-binding component of the ESCRT-1 endosomal sorting complex. Subsequent stimulation or homotypic adhesion promotes membrane translocation of Itch and the related protein Nedd4, resulting in degradation of two key signaling proteins, PLC-γ and PKCθ. T cells from Itch- and Cbl-b-deficient mice are resistant to anergy induction. Anergic T cells show impaired Ca²⁺ mobilization after TCR triggering and are unable to maintain a mature immunological synapse, instead showing late disorganization of the outer LFA-1-containing ring.

Accordingly, in one aspect, the invention includes a method of identifying an anergy modulating agent, comprising: (a) providing an E3 ubiquitin ligase polypeptide, E3 ubiquitin ligase substrate polypeptide, and a test compound; (b) contacting the test compound, the ligase polypeptide, and the ligase substrate polypeptide together under conditions that allow the ligase polypeptide to bind or ubiquitinate the substrate polypeptide; and (c) determining whether the test compound decreases the level of binding or ubiquitination of the substrate polypeptide by the ligase polypeptide, relative to the level of binding or ubiquitination in the absence of the test compound. A decrease indicates that the test compound is an anergy modulating agent. In certain embodiments, the E3 ligase polypeptide is selected from the group consisting of: Itch, GRAIL, Cbl, Cbl-b, Cbl-b3, Aip4, and Nedd4, or a polypeptide that is substantially identical thereto. The E3 ligase polypeptide can comprise an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12 or a polypeptide that is substantially identical thereto. In certain embodiments, the substrate polypeptide is selected from the group consisting of: PLC-γ, PKCθ, and RasGAP, or a polypeptide that is substantially identical thereto. The substrate polypeptide can comprise an amino acid sequence selected from the group consisting of SEQ ID NO:13, SEQ ID NO:14, SEQ ID NO:15, SEQ ID NO:16, SEQ ID NO:17, and SEQ ID NO:18 or a polypeptide that is substantially identical thereto.

In other embodiments, the method further includes (d) determining whether the agent reduces anergy in an immune cell (e.g. a T cell or a B cell) in vivo or in vitro and/or optimizing the pharmacological activity of the agent using modeling software and/or medicinal chemistry. In some embodiments, the test compound is cell-permeant.

In further embodiments, the ligase polypeptide is Itch and the substrate polypeptide is PLC-γ, or the ligase polypeptide is Itch and the substrate polypeptide is PKCθ, or the ligase polypeptide is Aip4 and the substrate polypeptide is PLC-γ, or the ligase polypeptide is Aip4 and the substrate polypeptide is PKCθ.

In another aspect, the invention includes a process for making an anergy modulating agent, the process includes manufacturing the agent identified using any one of the methods disclosed herein for identifying an anergy modulating agent. In one embodiment, an anergy modulating composition can be made by combining an anergy modulating agent manufactured according to the processes disclosed herein with a pharmaceutically acceptable carrier, to thereby manufacture an anergy modulating composition. In another embodiment, an anergy modulating composition can be combined into a pharmaceutical composition suitable for administration to an animal via a route selected from the group consisting of oral, parenteral, topical, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrasternal.

In another aspect, the invention includes a method of identifying an anergy modulating agent, comprising: (a) providing a test compound and a polypeptide selected from the group consisting of: Itch, Aip4, GRAIL, Cbl, Cbl-b, Cbl-b3, Nedd4, PLC-γ and PLCθ, or a biologically active fragment thereof; (b) contacting the test compound and the polypeptide or fragment thereof under conditions that allow the test compound to bind the polypeptide or fragment thereof; (c) determining whether the test compound binds the polypeptide or fragment thereof; and (d) determining whether the test compound reduces anergy in an immune cell (e.g. a T cell or a B cell) in vivo or in vitro, wherein a test compound that reduces anergy is an anergy modulating agent. In another embodiment, the method also includes optimizing the pharmaceutical activity of the agent using modeling software and/or medicinal chemistry.

In another aspect, the invention includes a method of identifying an anergy modulating agent, comprising: (a) providing a test compound and a polypeptide comprising Itch, Aip4, or a HECT fragment of Itch or Aip4; (b) contacting the test compound and the polypeptide under conditions that allow the test compound to interact with the polypeptide; (c) contacting the polypeptide with a reaction mix comprising E1, E2, tagged ubiquitin, and ATP; and (d) determining whether the test compound prevents the autoubiquitination of the polypeptide in the presence of the reaction mix; wherein a test compound that prevents the autoubiquitination of the polypeptide is an anergy modulating agent. In another embodiment, the method includes: (e) determining whether the agent reduces anergy in an immune cell (e.g., T cell or B cell) in vivo or in vitro. In some embodiments, the tagged ubiquitin includes a biotin, epitope, or fluorescent tag. In some embodiments, the E2 is UbCH7. In some embodiments, the method also includes optimizing the pharmacological activity of the agent using modeling software and/or medicinal chemistry.

In another aspect, the invention includes a method of identifying an anergy modulating agent, comprising: (a) contacting a test compound and an E3 ubiquitin ligase polypeptide under conditions that allow the test compound to interact with the ligase polypeptide; (b) contacting the ligase polypeptide with a reaction mix comprising E1, E2, tagged ubiquitin, ATP, and an E3 ubiquitin ligase substrate polypeptide; and (c) determining whether the test compound inhibits the ligase polypeptide from transubiquitinating the substrate polypeptide in the presence of the reaction mix, wherein a test compound that inhibits transubiquitination is an anergy modulating agent. In some embodiments, the E2 is UbCH7. In one embodiment, the method also comprises: (d) determining whether the agent reduces anergy in an immune cell (e.g., T cell or B cell) in vivo or in vitro. In certain embodiments, the test compound is cell-permeant.

In another aspect, the invention features a method of inhibiting anergy in a cell or patient, which comprises administering to a cell or patient an agent capable of inhibiting the production, activation, activity, or substrate binding ability of an anergy associated E3 ubiquitin ligase, in an amount sufficient to inhibit anergy in the cell or patient. In some embodiments, the ligase is selected from the group consisting of Itch, Grail, Cbl, Cbl-b, Cbl-b3, AIP4, and Nedd4, or a polypeptide that is substantially identical thereto. In certain embodiments, the agent is administered to a patient in need of treatment that inhibits anergy in the patient's immune cells. In some cases the patient is suffering from cancer. In some of those cases the agent is administered as a part of a combination therapy for cancer.

In another aspect, the invention includes a method identifying an agent that inhibits protein-protein interaction between an anergy associated E3 ubiquitin ligase and an E3 ubiquitin ligase substrate, and the method comprises: (a) providing an E3 ubiquitin ligase polypeptide, E3 ubiquitin ligase substrate polypeptide, and a test compound, wherein the ligase polypeptide or the substrate polypeptide is labeled; (b) contacting the ligase polypeptide, the substrate polypeptide, and the test compound with each other; and (c) determining the amount of label bound to the unlabeled polypeptide, wherein a reduction in the amount of label that binds the unlabeled polypeptide indicates that the test compound is an agent that inhibits protein-protein interaction between an anergy associated E3 ubiquitin ligase and an E3 ubiquitin ligase substrate.

In another aspect, the invention includes a method of identifying an agent that inhibits protein-protein interaction between an anergy associated E3 ubiquitin ligase and an E2 ubiquitin ligase, comprising: (a) providing E3 ubiquitin ligase polypeptide, E2 ubiquitin ligase polypeptide, and a test compound, wherein the E3 ligase polypeptide or the E2 ubiquitin ligase polypeptide is labeled; (b) contacting E3 ubiquitin ligase polypeptide, the E2 ubiquitin ligase polypeptide, and the test compound with each other; and (c) determining the amount of label bound to the unlabeled ligase polypeptide, wherein a reduction in the amount of label that binds the unlabeled ligase indicates that the test compound is an agent that inhibits protein-protein interaction between an anergy associated E3 ubiquitin ligase and an E2 ubiquitin ligase.

In yet another aspect, the invention includes a method for decreasing a protein-protein interaction between an E3 ubiquitin ligase and an E3 ubiquitin ligase substrate, comprising: contacting an anergy associated E3 ubiquitin ligase with an agent that decreases an interaction between the anergy associated E3 ubiquitin ligase and an E3 ubiquitin ligase substrate, such that the protein-protein interaction between the ligase and the substrate is decreased. In some embodiments, the ligase is Itch and the substrate is PLC-γ, or the ligase is Itch and the substrate is PKCθ, or the ligase is Aip4 and the substrate is PLC-γ, or the ligase is Aip4 and the substrate is PKCθ.

In another aspect, the invention includes a method of evaluating a test compound for an ability to modulate anergy, and the method comprises: (a) contacting an immune cell with a test compound and (b) determining whether the test compound modulates transcription of at least one anergy associated E3 ubiquitin ligase gene, wherein a test compound that reduces transcription is an anergy modulating agent. In one embodiment, the method also includes (c) determining whether the agent reduces tolerance induction in T or B cells in vivo or in vitro. In some embodiments E3 ligase gene encodes a ligase selected from the group consisting of Itch, Grail, Cbl, Cbl-b, Cbl-b3, AIP4, and Nedd4, or a polypeptide that is substantially identical thereto.

In some embodiments, the methods disclosed herein for identifying an anergy modulating agent or the methods disclosed herein for identifying an agent that inhibits protein-protein interactions can be performed using high-throughput screening methods.

In one aspect, the invention includes an agent identified by any one of the methods disclosed herein for identifying an anergy modulating agent.

In another aspect, the invention includes a vector comprising an isolated nucleic acid molecule encoding an anergy associated polypeptide or biologically active fragment thereof. In some embodiments, the anergy associated polypeptide is selected from the group consisting of Itch, GRAIL, Cbl, Cbl-b, Cbl-b3, Aip4, Nedd4, PLC-γ, PKCθ, and RasGAP, or a polypeptide that is substantially identical thereto. An anergy associated polypeptide can comprise an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, and SEQ ID NO:18, or a polypeptide that is substantially identical thereto. In some embodiments the vector is contained by a host cell.

In one aspect the invention includes a host cell that contains an exogenously introduced isolated nucleic acid molecule capable of expressing an anergy associated polypeptide or biologically active fragment thereof.

Unless otherwise defined, all technical and scientific terms used herein have the same meaning as commonly understood by one of ordinary skill in the art to which this invention belongs. Although methods and equipment or software similar or equivalent to those described herein can be used in the practice of the present invention, suitable methods, equipment, and software are described below. All publications and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features and advantages of the invention will be apparent from the description and drawings, and from the claims.

### DESCRIPTION OF DRAWINGS

FIG. 1A illustrates the Aip4 amino acid sequence.

FIG. 1B illustrates the Itch amino acid sequence.

FIG. 2A illustrates the human Nedd4 amino acid sequence.

FIG. 2B illustrates the mouse Nedd4 amino acid sequence.

FIG. 3A illustrates the human Cbl amino acid sequence.

FIG. 3B illustrates the mouse Cbl amino acid sequence.

FIG. 4A illustrates the human Cbl-b amino acid sequence.

FIG. 4B illustrates the mouse Cbl-b amino acid sequence.

FIG. 5A illustrates the human Cbl-3 amino acid sequence.

FIG. 5B illustrates the mouse Cbl-3 amino acid sequence.

FIG. 6A illustrates the human Grail amino acid sequence.

FIG. 6B illustrates the mouse Grail amino acid sequence.

FIG. 7A illustrates the human PLC-γ amino acid sequence.

FIG. 7B illustrates the mouse PLC-γ amino acid sequence.

FIG. 8A illustrates the human PKCθ amino acid sequence.

FIG. 8B illustrates the mouse PKCθ amino acid sequence.

FIG. 9A illustrates the human RasGAP amino acid sequence.

FIG. 9B illustrates the mouse RasGAP amino acid sequence.

FIG. 10 is an immunoblot illustrating that E6AP is capable of auto-ubiquitination.

FIG. 11 is an SDS-polyacrylamide gel illustrating that the HECT domain of E6AP suffices for self-ubiquitination.

FIG. 12 is an SDS-polyacrylamide gel illustrating that AIP4 and E6AP self-ubiquitinate in vitro.

FIG. 13 is a diagram illustrating the steps of an exemplary assay to identify inhibitors of E3 ligase activity.

FIG. 14A is a group of immunoblots illustrating changes in signaling proteins in anergic T cells. T cell anergy was induced by treating the Th1 cell clone D5 with (+) or without (−) 1 μM ionomycin for 16 hours. The cells were washed to remove the ionomycin, and incubated at higher cell density for 1-2 hours at 37° C. Whole cell extracts were analyzed by Western blotting.

FIG. 14B is a composite picture of an immunoblot illustrating the effect of ionomycin and high cell density on PLC-γ1 levels in a D5 Th1 clone. Anergy was induced by treating the D5 Th1 clone with 1 μM ionomycin for 16 hours. Cells were washed to remove the ionomycin and incubated at higher cell density for 1 hour at 37° C. Extracts were assayed for PLC-γ1 levels by immunoblotting. Extracts were prepared either directly (lanes 1, 2) or after resuspension at high cell density and incubation for 1 hr (lanes 3, 4).

FIG. 14C is a chart and immunoblot illustrating the effect of restimulation on PLC-γ1 levels in a D5 Th1 clone. Cells were prepared as described in FIG. 14B, and restimulated with anti-CD3, anti-CD3/anti-CD28, ionomycin or PMA/ionomycin for 1 h.

FIG. 14D is a bar graph and immunoblot illustrating the extent of anergy induction in a proliferation assay, and the extent of decrease in PLC-γ1 levels after the step of incubation at high cell density, in parallel in a single culture of untreated (−) and ionomycin-pretreated (+) D5 cells. Cells were prepared as described for FIG. 14B.

FIG. 14E is a set of graphs illustrating calcium mobilization in anergic T cells in response to TCR stimulation. Primary Th1 cells from 2B4 mice were either left untreated (top panel) or pretreated with ionomycin for 16 hours (lower panel) prior to fura-2 labeling and [Ca]i imaging.

FIG. 15A is a flowchart for generating anergic and activated primary Th1 cells, and a group of immunoblots illustrating the effect of anergy and activation on the level of various proteins in the cell. CD4+ cells were isolated and differentiated into Th1 cells in vitro, then stimulated with either plate-bound anti-CD3 to induce anergy or with a combination of anti-CD3 and anti-CD28 to induce productive activation. In both cases the cells go through a phase of active proliferation but cells that only received anti-CD3 stimulation respond much less to subsequent restimulation than cells that were stimulated with both anti-CD3 and anti-CD28. This protocol was chosen in preference to anergy induction by sustained treatment with ionomycin as in D5 T cells, because levels of homotypic adhesion were variable in ionomycin-pretreated primary Th1 cells, depending on mouse strain and exact conditions of Th1 differentiation and ionomycin pretreatment employed. Equal numbers of anergized (right lane) and activated (left lane) T cells were analyzed by immunoblotting for protein levels of the indicated proteins. Diminished protein levels were observed for PLC-γ1, PKCθ, RasGAP and Lck but not for PLC-γ2.

FIG. 15B is a chart and a group of immunoblots illustrating that Nedd4 is preactivated for membrane localization in T cells subjected to sustained Ca2+ signaling. D5 cells were left untreated (upper panel) or pretreated with ionomycin for 16 hrs (lower panel), then stimulated for 1 h with either anti-CD3 or anti-CD3/anti-CD28. The cells were fractionated, and fractions were analyzed by immunoblotting for levels of Nedd4 protein.

FIG. 15C is a chart and immunoblot illustrating the upregulation of Itch protein in anergic D5 Th1 cells. Cells were left resting (lane 4) or were stimulated for 16 hrs with 0.25 or 1 μg/ml plate-bound anti-CD3, without (lanes 2-4) or with costimulation through 2 μg/ml anti-CD28 (lane 1). Stimulation increases cell size and leads to an overall increase of cytoplasmic protein as compared to resting conditions (compare lanes 1-3 with lane 4). At low anti-CD3 concentrations, stimulation through the TCR alone induces a considerably greater increase in Itch protein levels relative to combined anti-CD3/anti-CD28 stimulation (compare lane 3 with lane 1). High concentrations of anti-CD3 (lane 2) do not induce the increase, a finding best explained by the antagonism between Ca²⁺ and PMA-stimulated signaling pathways for upregulation of anergy-associated genes. Concurrent PMA stimulation counters the ability of Ca²⁺ signaling to upregulate most anergy-associated genes; similarly, low doses of anti-CD3 which predominantly induce Ca²⁺ influx upregulate the anergy-associated genes, but this is not observed if cells are stimulated with higher doses of anti-CD3 which activate other signaling pathways as well. Although a loading control was not available for this experiment, Itch and Cbl-b levels were also upregulated in the experiment of FIG. 15A, in which PLC-γ1 and PKCθ levels decline but PLC-γ2 levels are not changed.

FIG. 15D is a pair of immunoblots illustrating that Itch is a target of the AP-1-independent transcriptional program driven by NFAT. NIH3T3 cells were twice infected with control IRES GFP-retrovirus or retrovirus encoding CA-NFAT1-RIT, a constitutively-active NFAT1 harboring mutations within the AP-1 interaction surface (RIT). Two days after the last infection, extracts were prepared and analyzed for Itch as well as Nedd4 expression by western blotting. The ratio of specific band densities for Itch versus Nedd4 in duplicate experiments was normalized to the ratio observed in the control infection and is depicted as Itch/Nedd4.

FIG. 16A is a chart and a set of immunoblots illustrating calcineurin-dependent degradation of target proteins in anergic T cells. D5 T cells were treated with ionomycin (iono), cyclosporin A (CsA) or both for 16 hrs, then washed and incubated at increased cell density for 1 hr. Cell extracts were prepared and analyzed by immunoblotting for the indicated proteins or for the extent of ubiquitin modification of total protein in the lysates. The faster-migrating band in the PKCθ immunoblot (asterisk) is the original ZAP70 signal on the same blot, which was reprobed without prior stripping.

FIG. 16B is a set of immunoblots illustrating the effect of anti-CD3 stimulation on CD4T cells. CD4 T cells from DO11.10 mice or mice that were orally tolerized with ovalbumin in the drinking water were purified and subjected to anti-CD3 stimulation for the indicated times. Extracts were analyzed by immunoblotting for PLC-γ1, PKCθ and Lck proteins. T cells from tolerized mice showed an early decrease in PLC-γ1 and PKCθlevels under these conditions (right panel), suggesting that degradation was primarily associated with the initial phase of TCR stimulation. In contrast T cells from untreated mice showed a decline in the levels of these proteins at later times (2-3 h; left panel), suggesting that a downregulatory program similar to anergy might be turned on normally after late times of T cell activation. Note that this downregulation was not observed in the pulse-chase shown in (16C); we attribute this to a difference in the strength of stimulus in the two experiments since bead-bound anti-CD3 was used in (A) while plate-bound anti-CD3 was used in (16C).

FIG. 16C is a set of autoradiographs illustrating the time course of degradation of PKCθ in CD4T cells. CD4 T cells from control or by gastric injection tolerized DO11.10 mice were pulse labeled with 35S-cysteine/methionine, then washed and incubated for the indicated times with complete media in the presence of plate bound anti-CD3. Cell extracts were immunoprecipitated with antibodies against PKCθ and analyzed by autoradiography.

FIG. 16D is a set of graphs illustrating decreased Ca²⁺ mobilization in T cells made orally tolerant to high-dose antigen in vivo. CD4 T cells were isolated from DO11.10 TCR transgenic mice that were left untreated (top panel) or received gastric injections (g.i.) of ovalbumin to induce T cell tolerance (bottom panel), and labeled with fura-2. After an observation period of 100 sec, streptavidin was added to induce TCR crosslinking (TCR); at 600 sec, ionomycin (iono) was added to identify responsive cells (arrows). Ca²⁺ mobilization was monitored by time-lapse video microscopy. Individual (gray) and averaged (black) traces from ˜100 CD4+ and ionomycin-responsive single cells are shown. The in vivo-tolerized T cells show very low levels of Ca2+ mobilization in response to TCR crosslinking.

FIG. 17A is a schematic representation of the domain organization of PLC-γ1, PKCθ, RasGAP, Itch, and Nedd4. Domains indicated are PH (pleckstrin homology); EF hand; X and Y, the split catalytic region of PLC-γ1; SH2 and SH3, src homology type 2 and 3; and C1 and C2 domains. WW, protein interaction domains; HECT, catalytic domain involved in ubiquitin transfer.

FIG. 17B is a chart and a set of immunoblots illustrating physical interaction of Nedd4 and Itch with PLC-γ1. AU-tagged PLC-γ1 was co-expressed in HEK 293 cells with myc-tagged Itch or a myc-tagged Nedd4 isoform (accession number KIAA0093). Anti-myc immunoprecipitates (top two panels) or whole cell lysates (bottom two panels) were analyzed by immunoblotting for levels of the indicated proteins. PLC-γ1 in immunoprecipitates was detected with the cocktail of monoclonal antibodies (Upstate) (top panel).

FIG. 17C is a chart and a set of immunoblots illustrating that Itch induces mono-, di- and poly-ubiquitination of PLC-γ1. HEK 293 cells were transfected in duplicate with expression vectors coding for HA-tagged ubiquitin, AU.1-tagged PLC-γ1 and/or myc-tagged Itch as indicated, and one culture of each pair was stimulated with 3 μM ionomycin for 30 min before cell extraction. Cell extracts were immunoprecipitated with AU.1 antibodies and analyzed for ubiquitin-modified or total immunoprecipitated PLC-γ1 (upper two panels), or were directly analyzed for PLC-γ1 and Itch expression by immunoblotting (lower two panels).

FIG. 17D is a set of immunoblots illustrating that Itch and Nedd4 promote PLC-γ1 degradation. HEK 293 cells were transfected and stimulated with ionomycin as indicated. A comparison of endogenous and transfected Nedd4 or Itch protein levels is shown in the lower panel.

FIG. 17E is a set of immunoblots illustrating changes in Nedd4, Itch and LAT proteins in various cell fractions. D5 cells were left untreated (−) or were stimulated with ionomycin (+) for 16 hrs, then washed and incubated at increased cell density for 2 hours. Cell extracts were prepared by lysis in hypotonic buffer and fractionated (see Examples). One-fourth of the supernatant from each centrifugation step (cytoplasm, detergent soluble and detergent insoluble fractions) was analyzed for Nedd4, Itch, and LAT proteins.

FIG. 17F is a chart and set of immunoblots illustrating that the proteasome inhibitor MG132 does not inhibit PLC-γ1 degradation and promotes accumulation of a modified form of PKCθ. D5 T cells were treated with ionomycin for 16 h, then washed and incubated in the absence or presence of 10 μM MG132. Extracts were immunoblotted for PLC-γ1 and PKCθ. The mechanism by which MG132 increases the level of mono-ubiquitinated PKCθ is possibly secondary: blocking proteasome function may lead to an increase in the overall amount of ubiquitin-conjugates in the cell, thus tending to saturate deubiquitinating enzymes and decreasing the efficiency of deubiquitination of any individual substrate.

FIG. 17G is a set of immunoblots illustrating that PKCθ becomes monoubiquitinated in cells subjected to sustained Ca2+ signaling. 10⁸ D5 cells were either left untreated or pretreated with ionomycin, lysed and immunoprecipitated with antibodies to PKCθ in RIPA buffer. The immunoprecipitates were analyzed for ubiquitin modification by immunoblotting.

FIG. 18A is a chart and a set of immunoblots illustrating the upregulation of Itch, Cbl-b and Tsg101 in anergic T cells. D5 Th1 cells were left resting or were stimulated with ionomycin, cyclosporin A or both. RIPA extracts were probed for Itch, Tsg101, Cbl-b and Nedd4 protein in immunoblots, and the intensities were quantified by NIH IMAGE Quant and corrected for the background within the specific lane.

FIG. 18B is a bar graph illustrating the effect of ionomycin and cyclosporin A on mRNA levels of various proteins in D5 cells. D5 cells were left untreated or stimulated with ionomycin or ionomycin and cyclosporin A for 10 hours, and mRNA levels of Itch, cbl-b, Grail and PLC-γ1 were evaluated by real-time RT-PCR, normalizing to L32-levels. The ratio of mRNA levels in ionomycin-treated or ionomycin/CsA-treated to untreated cells is shown.

FIG. 19A is a set of graphs illustrating an assessment of ionomycin-induced T cell unresponsiveness. Ionomycin-induced unresponsiveness was assessed in primary Th1 cells by intracellular cytokine staining for IL-2 after restimulation with anti-CD3/anti-CD28.

FIG. 19B is a set of images illustrating the distribution of ICAM-1 (red) and I-Ek-MCC (green) molecules in T cell-bilayer contact zones as captured at different time points in control and ionomycin-treated cells. Control and ionomycin-treated cells were incubated for 40 minutes on planar phospholipid bilayers containing Oregon green-labeled I-EK/agonist moth cytochrome C peptide complexes and Cy₃-labelled ICAM-1.

FIG. 19C is a set of images illustrating the cell-bilayer contacts, seen as dark areas on IRM images, recorded after 10, 20 and 30 minutes of incubation in control and anergized Th1 cells.

FIG. 20A illustrates the human Tsg101 amino acid sequence.

FIG. 20B illustrates the mouse Tsg101 amino acid sequence.

FIG. 21 is a set of autoradiograms illustrating calcineurin-dependent degradation of PKCθ in anergic T cells. Th1 cells from BALB/c mice were left untreated or pretreated with ionomycin for 16 h, pulse-labeled for 2 h with ³⁵S cysteine/methionine, washed and stimulated with plate-bound anti-CD3 antibody during the indicated chase periods. PKCθ immunoprecipitates were analyzed by autoradiography.

FIG. 22 is a set of images and a bar graph illustrating the role of PLC-γ1 in synapse stability. Involvement of PLC-γ1 in synapse stability was evaluated by allowing mature T cell synapses to form, then adding weak (U73343) or strong (U73122) PLC-γ1 inhibitors. The graph shows the percentage of cells with mature synapses relative to the same cells before addition of inhibitors.

FIG. 23 is a bar graph illustrating that naïve T cells from Itch−/− and Cbl-b−/− mice are resistant to ionomycin-induced anergy. Since Itch−/− and Cbl-b−/− mice have an age- and strain-dependent autoimmune phenotype, we repeated the experiment shown in FIG. 18C with purified naïve T cells to exclude the possibility that the lack of anergy induction observed with Itch−/− and Cbl-b−/− CD4 T cells reflected hyperproliferation of preactivated T cells. CD4 T cells isolated from spleen of wild-type, Cbl-b−/− and Itch−/− mice were selected for CD62L expression by magnetic selection (MACS, Miltenyi Biotec, Auburn, Calif.). The cells were left untreated or stimulated for 16 h with 50 ng/ml ionomycin, washed and stimulated with anti-CD3/anti-CD28. Proliferative responses were measured by ³H-thymidine incorporation.

FIG. 24 is a bar graph illustrating results obtained using an assay as described in the present specification.

FIGS. 25A-D are a set of experimental results comparing anergy induction in cells obtained from mice of three genotypes: Wild-Type, Cblb^{-/-}, and Itch^{-/-}. FIG. 25A is a histogram quantifying the proliferation responses of cells from the three mice. FIG. 25B is an immunoblot showing the breakdown of PLC-γ in response to anergy stimulus in cells from the three mice. FIG. 25C is an immunoblot showing the breakdown of PKC-θ in response to anergy stimulus in cells from the three mice. FIG. 25D is a series of images comparing synapse disintegration following anergy stimulus.

### DETAILED DESCRIPTION

In order that the present invention may be more readily understood, certain terms are first defined. Additional definitions are set forth throughout the detailed description.

The term “tolerance,” as used herein, refers to a down-regulation of at least one element of an immune response, for example, the down-regulation of a humoral, cellular, or both humoral and cellular responses. The term tolerance includes not only complete immunologic tolerance to an antigen, but also to partial immunologic tolerance, i.e., a degree of tolerance to an antigen that is greater than what would be seen if a method of the invention were not employed.

“Cellular tolerance,” or “anergy,” refers to downregulation of at least one response of an immune cell, e.g., a B cell or a T cell. Such downregulated responses may include, e.g., decreased proliferation in response to antigen stimulation, decreased cytokine (e.g., IL-2) production; and others.

As used herein, an “E3 ubiquitin ligase polypeptide” is an E3 ubiquitin ligase, or a biologically active fragment of such an E3 ubiquitin ligase, involved in anergy that can bind or ubiquitinate an E3 ubiquitin ligase substrate.

An “E2 ubiquitin ligase polypeptide” is an E2 ubiquitin ligase, or a biologically active fragment of such an E2 ubiquitin ligase, involved in anergy.

As used herein, an “E3 ubiquitin ligase substrate polypeptide” is an E3 ubiquitin ligase substrate, or a biologically active fragment of such a substrate, that can be bound or ubiquitinated by an “E3 ubiquitin ligase polypeptide.”

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded DNA.

The term “isolated or purified nucleic acid molecule” includes nucleic acid molecules that are separated from other nucleic acid molecules that are present in the natural source of the nucleic acid. For example, with regard to genomic DNA, the term “isolated” includes nucleic acid molecules that are separated from the chromosome with which the genomic DNA is naturally associated. An “isolated” nucleic acid can be free of sequences that flank the endogenous nucleic acid (i.e., sequences located at the 5′ and/or 3′ ends of the nucleic acid) in the genomic DNA of the organism from which the nucleic acid is obtained or derived (e.g., synthesized) from. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which flank the endogenous nucleic acid molecule in genomic DNA of the cell from which the nucleic acid is derived. The term therefore includes, for example, a recombinant DNA which is incorporated into a vector (e.g., an autonomously replicating plasmid or virus), or into the genomic DNA of a prokaryote or eukaryote. The term also includes a recombinant DNA that exists as a separate molecule (e.g., a cDNA or a genomic DNA fragment produced by PCR or restriction endonuclease treatment) independent of other sequences. It also includes a recombinant DNA that is part of a hybrid gene encoding additional polypeptide sequences. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A “substantially identical” nucleic acid means a nucleic acid sequence that encodes a polypeptide differing only by conservative amino acid substitutions, e.g., substitution of one amino acid for another of the same class (e.g., valine for leucine or isoleucine, arginine for lysine, etc.) or by one or more non-conservative substitutions, deletions, or insertions located at positions of the amino acid sequence which do not destroy the function of the polypeptide. A “substantially identical” polypeptide means a polypeptide differing only by conservative amino acid substitutions, e.g., substitution of one amino acid for another of the same class (e.g., valine for glycine, arginine for lysine, etc.) or by one or more non-conservative substitutions, deletions, or insertions located at positions of the amino acid sequence which do not destroy the function of the polypeptide. The terms “peptide”, “polypeptide”, and “protein” are used interchangeably herein.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue can be replaced with another amino acid residue from the same side chain family.

Homology is typically measured using sequence analysis software (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). Such software matches similar sequences by assigning degrees of homology to various substitutions, deletions, and other modifications.

A “substantially pure” preparation or a preparation that is “substantially free” of other material is a preparation that contains at least 60% by weight (dry weight) the compound of interest, e.g., a candidate compound or agent described herein. Preferably the preparation is at least 75%, more preferably at least 90%, and most preferably at least 99%, by weight the compound of interest. Purity can be measured by any appropriate method, e.g., column chromatography, polyacrylamide gel electrophoresis, or HPLC analysis.

By “purified antibody” is meant antibody that is at least 60%, by weight, free from the proteins and naturally-occurring organic molecules with which it is naturally associated. The preparation can be at least 75%, e.g., at least 90%, or at least 99%, by weight, antibody.

The terms “therapeutically effective amount” and “effective to treat,” as used herein, refer to an amount or concentration of a compound or pharmaceutical composition described herein utilized for a period of time (including acute or chronic administration and periodic or continuous administration) that is effective within the context of its administration for causing an intended effect or physiological outcome. A therapeutically effective amount of a compound or pharmaceutical composition may vary according to factors such as the disease state, age, sex, and weight of the individual, and any other variable known to those of skill in the medicinal field.

The term “patient” is used throughout the specification to describe an animal, human or non-human, to whom treatment according to the methods of the present invention is provided. Veterinary applications are clearly contemplated by the present invention. The term includes but is not limited to birds, reptiles, amphibians, and mammals, e.g., humans, other primates, pigs, rodents such as mice and rats, rabbits, guinea pigs, hamsters, cows, horses, cats, dogs, sheep and goats. Preferred subjects are humans, farm animals, and domestic pets such as cats and dogs. The term “treat(ment),” is used herein to denote delaying the onset of, inhibiting, alleviating the effects of, or prolonging the life of a patient.

The terms “activate,” “induce,” “inhibit,” “elevate,” “increase,” “decrease,” “reduce,” or the like, denote quantitative differences between two states, e.g., a statistically significant difference, between the two states.

### Tolerance Induction

The present invention is based, in part, on evidence disclosed herein for a complex multi-step programme in which T cell anergy is imposed by degradation of key signaling proteins that act proximal to the TCR. Without intending to be bound by theory, in the first step of the programme, Ca²⁺/calcineurin signaling appears to increase mRNA and protein levels of three distinct E3 ubiquitin ligases, Itch, Cbl-b and Grail. Ca²⁺/calcineurin signaling also appears to increase mRNA and protein levels of the ubiquitin receptor Tsg101. Tsg101 is the key ubiquitin-binding component of the endosomal sorting complex, ESCRT-1, which sorts proteins associated with endosomal membranes into small internal vesicles of multivesicular bodies, which are later degraded when they fuse with lysosomes.

The second step of the programme appears to be the degradation of key signaling proteins, which is implemented upon T cell-APC contact. By ubiquitinating the TCR, Cbl-b promotes its internalisation and retention in endosomes. At the same time, Itch moves to detergent-insoluble membrane fractions (“raft” membranes, endosomal membranes, or both) where it colocalizes with and mono-ubiquitinates two key signalling proteins, PLC-γ1 and PKCθ, promoting their interaction with Tsg101 and targeting them for lysosomal degradation. As a result of this multistep programme, degradation of PLC-yl and PKCθ in anergic T cells can be dependent on Ca²⁺/calcineurin signalling.

Anergic T cells show impaired Ca²⁺ mobilization after TCR triggering and are unable to maintain a mature immunological synapse. Instead they show late disorganization of the outer LFA-1-containing ring and displaying a “migratory” phenotype resembling that of cells that do not receive a TCR-mediated “stop” signal. It is likely that synapse disorganization initially arises because degradation of active PLC-γ1 and PKCθ leads to diminished TCR/LFA-1 signaling. Once this happens the mature synapse cannot be maintained and the inability to sustain stable APC contact further reduces the antigen responses of anergic T cells. Genetic evidence for the involvement of Itch and Cbl-b in T cell anergy includes the finding that Itch^{-/-} and Cbl-b^{-/-} T cells are resistant to anergy induction, especially at low doses of ionomycin (see Example 3, below).

### Screening Methods

The present invention provides screens for identifying compounds (e.g., small organic or inorganic molecules (e.g., having a molecular weight of less than 2500 Da), polypeptides (e.g., an antibody such as an intrabody), peptides, peptide fragments, peptidomimetics, antisense oligonucleotides, or ribozymes) capable of inhibiting the production, activity, activation, and/or substrate binding ability of anergy-associated E3 ubiquitin ligases (i.e., Itch, Cbl-b, Cbl, Cbl-3, Grail, Nedd4, and Aip4). The screens can be performed in a high-throughput format. Such inhibitors can modulate anergy induction and are useful, e.g., to interfere with the documented ability of tumors to induce tolerance in T cells. Such compounds can be therapeutically useful in boosting the immune response to tumors, and might be particularly useful for eliminating surviving tumor cells after chemotherapy. Such compounds may also be therapeutically useful in boosting the immune response to a pathogenic infection, e.g., a viral, bacterial, or parasitic infection.

As used herein, the term “anergy-associated” nucleic acids or their corresponding protein products are those whose expression is modulated (e.g., increased or decreased) in response to calcium induced signaling. Changes in the expression of anergy-associated nucleic acids or proteins may be a causative factor in inducing, promoting, and/or maintaining tolerance or anergy (i.e., an anergy-inducing nucleic acid), or may simply be a result of the anergic state (i.e., an anergy-induced nucleic acid). Anergy-associated gene products may have a negative feedback on the production of an immune response, e.g., by uncoupling an antigen receptor, e.g., a T or a B cell receptor, from the proximal signaling pathways.

Anergy-associated nucleic acids and proteins include anergy-associated E3 ubiquitin ligases (alternatively referred to herein as “E3 ligase(s),” “E3 ubiquitin ligase(s)” and “ligase(s)”), e.g., Itch, Cbl-b, Cbl, Cbl-3, Grail, Nedd4, and atrophin-1 interacting protein 4 (Aip4), the nucleic acid and amino acid sequences for which are known and described herein. Also included within the terms (i.e., “anergy associated E3 ubiquitin ligase” and “ligase”) are biologically active (e.g., substrate binding and/or ubiquitinating, and/or E2 binding), domains or fragments of the of the E3 ubiquitin ligase. An example of such a domain or fragment is the so-called HECT domain of Itch and Aip4. Also included are chimeric recombinant proteins, e.g., E3 ubiquitin ligase or a biologically active fragment thereof fused to another peptide or protein such that biological activity is preserved. The E3 ubiquitin ligase or fragment thereof can be natural, recombinant or synthesized. In certain embodiments, the E3 ubiquitin ligase can be from, e.g., a mammal, e.g., a human, or yeast. An E3 ubiquitin ligase can be obtained, e.g., in cell extracts of cells that normally express E3 ubiquitin ligase, or by expressing recombinant E3 ubiquitin ligase protein in eukaryotic or prokaryotic cells.

The nucleic acid and amino acid sequences of human and mouse Itch, Cbl-b, Cbl, Cbl-3, Grail, Nedd4, and Aip4 are known and can be found at the National Center for Biotechnology Information (NCBI) database using GenBank accession numbers. The NCBI database is accessible on the World Wide Web at address ncbi.nlm.nih.gov. The GenBank accession numbers for the Itch nucleic acid and amino acid sequences are XM_{—}192925 and XP_{—}192925, respectively. The GenBank accession numbers for the Aip4 nucleic acid and amino acid sequences are NM_{—}031483 and NP_{—}113671, respectively. The GenBank accession numbers for Nedd4 nucleic acid and amino acid sequences are XM_{—}046129 and XP_{—}046129, respectively for human Nedd4, and NM_{—}010890 and NP_{—}035020, respectively for mouse Nedd4. The GenBank accession numbers for Cbl nucleic acid and amino acid sequences are NM_{—}005188 and NP_{—}005179, respectively, for human Cbl, and AK085140 and NP_{—}031645, respectively, for mouse Cbl. The GenBank accession numbers for Cbl-b nucleic acid and amino acid sequences are U26710 and Q13191, respectively, for human Cbl-b, and XM_{—}156257 and XP_{—}156257, respectively, for mouse (partial sequence) Cbl-b. The GenBank accession numbers for Cbl-3 nucleic acid and amino acid sequences are NM_{—}012116 and NP_{—}036248, respectively, for human Cbl-3, and NM_{—}023224 and NP_{—}075713, respectively for mouse Cbl-3. The GenBank accession numbers for Grail nucleic acid and amino acid sequences are NM_{—}024539 and NP_{—}078815, respectively, for human Grail, and NM_{—}023270 and NP_{—}075759, respectively, for mouse Grail.

Anergy associated nucleic acids and proteins also include anergy-associated E3 ubiquitin ligase substrate(s) (alternatively referred to herein as “ligase substrate(s)” and “substrate(s)”), e.g., phospholipase-C₁₋₇ (PLC-γ), protein kinase C-θ (PKCθ), the Ras GTPase-activating protein (RasGAP), Lck, ZAP-70, and the signalling subunits of the TCR/CD3 complex (e.g., CD3 epsilon, delta, and zeta). The nucleic acid and amino acid sequences for PLC-γ, PKCθ, RasGAP, Lck, ZAP-70, and the signalling subunits of the TCR/CD3 complex, are known and described herein. Also included within the terms are biologically active domains or fragments of the substrate capable of being bound and/or ubiquitinated by an anergy associated E3 ubiquitin ligase, i.e., Itch, Cbl-b, Cbl, Cbl-3, Grail, Nedd4, and/or Aip4, or fragments thereof. Also included are chimeric recombinant proteins, e.g., ligase substrate or a biologically active fragment thereof fused to another peptide or protein such that biological activity is preserved. The ligase substrate or biologically active fragment can be natural, recombinant or synthesized. In certain embodiments, the ligase substrate can be from, e.g., a mammal, e.g., a human, or yeast. The ligase substrate can be obtained, e.g., in cell extracts of cells that normally express ligase substrate, or by expressing recombinant ligase substrate protein in eukaryotic or prokaryotic cells.

The nucleic acid and amino acid sequences of PLC-γ, PKCθ, RasGAP, Lck, ZAP-70, and the signalling subunits of the TCR/CD3 are known and can be found at the NCBI database using GenBank accession numbers. The GenBank accession numbers for PLC-γ nucleic acid and amino acid sequences are NM_{—}002660 and NP_{—}002651, respectively, for human PLC-γ, and XM_{—}130636 and XP_{—}130636, respectively, for mouse PLC-γ. The GenBank accession numbers for PKCθ nucleic acid and amino acid sequences are NM_{—}002660 and NP_{—}006248, respectively, for human PKCθ, and NM_{—}008859 and NP_{—}032885, respectively, for mouse PKCθ. The GenBank accession numbers for RasGAP nucleic acid and amino acid sequences are NM_{—}002890 and NP_{—}002881, respectively, for human RasGAP, and NM_{—}145452 and NP_{—}663427, respectively, for mouse (partial sequence) RasGAP. The GenBank accession numbers for Lck nucleic acid and amino acid sequences are NM_{—}005356 and NP_{—}005347, respectively, for human Lck, and BC011474 and AAH11474, respectively, for mouse Lck. The GenBank accession numbers for ZAP-70 nucleic acid and amino acid sequences are NM_{—}001079 and NP_{—}001070, respectively, for human ZAP-70, and NM_{—}009539 and NP_{—}033565, respectively, for mouse ZAP-70. The GenBank accession numbers for CD3 epsilon nucleic acid and amino acid sequences are NM_{—}000733 and NP_{—}000724, respectively, for human CD3 epsilon, and NM_{—}007648 and NP_{—}031674, respectively, for mouse CD3 epsilon. The GenBank accession numbers for CD3 delta nucleic acid and amino acid sequences are NM_{—}000732 and NP_{—}000723, respectively, for human CD3 delta, and NM_{—}013487 and NP_{—}038515, respectively, for mouse CD3 delta. The GenBank accession numbers for CD3 zeta nucleic acid and amino acid sequences are NM_{—}000734 and NP_{—}000725, respectively, for human CD3 zeta, and NM_{—}031162 and NP_{—}112439, respectively, for mouse CD3 zeta.

Anergy associated nucleic acids and proteins also include the ubiquitin receptor Tsg101. The GenBank accession numbers for Tsg101 nucleic acid and amino acid sequences are NM_{—}006292 and NP_{—}006283, respectively for human Tsg101, and NM_{—}021884 and NP_{—}068684, respectively for mouse Tsg101.

Anergy associated nucleic acids and proteins also include nucleic acid sequences and amino acid sequences that are substantially identical to the anergy associated nucleic acids and proteins described herein, as well as homologous sequences.

By anergy associated protein fragment is meant some portion of, or a synthetically produced sequence derived from, the protein (e.g., the naturally occurring protein). In some embodiments, the fragment is less than about 150 amino acid residues, e.g., less than about 100, 50, 30, 20, 10, or 6 amino acid residues. The fragment can be greater than about 3 amino acid residues in length. Fragments include, e.g., truncated secreted forms, cleaved fragments, proteolytic fragments, splicing fragments, other fragments, and chimeric constructs between at least a portion of the relevant gene and another molecule. In some embodiments, the fragment is biologically active. The ability of a fragment to exhibit a biological activity of the anergy associated protein can be assessed by, e.g., its ability to ubiquitinate and/or bind (in the case of E3 ubiquitin ligases) ligase substrates, or to be ubiquitinated and/or bound (in the case of E3 ubiquitin ligase substrates) by E3 ubiquitin ligases. Also included are fragments containing residues that are not required for biological activity of the fragment or that result from alternative mRNA splicing or alternative protein processing events. Examples of useful fragments include those listed in Table 1, below.

TABLE 1
Exemplary anergy associated protein fragments

| gene | figure | SEQ ID NO | amino acid nos. | domain |
|---|---|---|---|---|
| mouse itch | 1B | 2 | 8-101 | C2 protein kinase C conserved region 2 |
| | | | 283-360 | homologous to splicing factor PRP40 |
| | | | 395-460 | |
| | | | 306-854 | HUL4 ubiquitin-protein ligase domain |
| | | | 499-854 | HECT ubiquitin-protein ligase domain |
| | | | 278-310 | WW domains |
| | | | 310-341 | |
| | | | 390-422 | |
| | | | 430-461 | |
| human itch | 1A | 1 | 10-111 | C2 protein kinase C conserved region 2 |
| | | | 291-368 | homologous to splicing factor PRP40 |
| | | | 403-468 | |
| | | | 314-862 | HUL4 ubiquitin-protein ligase domain |
| | | | 507-862 | HECT ubiquitin-protein ligase domain |
| | | | 286-318 | WW domains |
| | | | 318-349 | |
| | | | 398-430 | |
| | | | 438-469 | |
| human NEDD | 2A | 3 | 20-124 | C2 protein kinase C conserved region 2 |
| | | | 20-171 | homologous to calcium-dependent lipid-binding |
| | | | | protein |
| | | | 196-224 | WW domains |
| | | | 349-380 | |
| | | | 423-452 | |
| | | | 474-505 | |
| | | | 350-897 | HUL4 ubiquitin-protein ligase domain |
| | | | 427-504 | homologous to splicing factor PRP40 |
| | | | 543-899 | HECT ubiquitin-protein ligase domain |
| mouse NEDD | 2B | 4 | 6-73 | C2 protein kinase C conserved region 2 |
| | | | 144-172 | WW domains |
| | | | 296-328 | |
| | | | 351-382 | |
| | | | 297-774 | HUL4 ubiquitin-protein ligase domain |
| | | | 301-381 | homologous to splicing factor PRP40 |
| | | | 420-776 | HECT ubiquitin-protein ligase domain |
| human Cbl | 3A | 5 | 49-176 | Cbl N-terminal domain, binds phosphorylated |
| | | | | tyrosines |
| | | | 178-262 | Cbl EF hand-like domain |
| | | | 264-349 | Cbl SH2-like domain |
| | | | 373-434 | HRD ubiquitin ligase domain |
| | | | 381-423 | RING finger domain |
| | | | 861-894 | ubiquitin associated domain |
| mouse Cbl | 3B | 6 | 48-174 | Cbl N-terminal domain, binds phosphorylated |
| | | | | tyrosines |
| | | | 176-260 | Cbl EF hand-like domain |
| | | | 262-347 | Cbl SH2-like domain |
| | | | 358-415 | HRD ubiquitin ligase domain |
| | | | 363-404 | RING finger domain |
| | | | 847-884 | ubiquitin associated domain |
| human Cbl-b | 4A | 7 | 42-168 | Cbl N-terminal domain, binds phosphorylated |
| | | | | tyrosines |
| | | | 171-254 | Cbl EF hand-like domain |
| | | | 256-341 | Cbl SH2-like domain |
| | | | 365-419 | HRD ubiquitin ligase domain |
| | | | 371-415 | RING finger domain |
| | | | 933-969 | ubiquitin associated domain |
| mouse Cbl-b (partial) | 4B | 8 | 498-534 | ubiquitin associated domain |
| human Cbl-3 | 5A | 9 | 13-146 | Cbl N-terminal domain, binds phosphorylated |
| | | | | tyrosines |
| | | | 149-232 | Cbl EF hand-like domain |
| | | | 234-322 | Cbl SH2-like domain |
| | | | 350-421 | HRD ubiquitin ligase domain |
| | | | 325-401 | RING finger domain |
| mouse Cbl-3 | 5B | 10 | 16-145 | Cbl N-terminal domain, binds phosphorylated |
| | | | | tyrosines |
| | | | 148-231 | Cbl EF hand-like domain |
| | | | 234-318 | Cbl SH2-like domain |
| | | | 332-442 | HRD ubiquitin ligase domain |
| | | | 350-392 | RING finger domain |
| human Grail | 6A | 11 | 83-183 | protease-associated domain |
| | | | 268-385 | HRD ubiquitin ligase domain |
| | | | 274-321 | RING finger domain |
| mouse Grail | 6B | 12 | 83-183 | protease-associated domain |
| | | | 268-368 | HRD ubiquitin ligase domain |
| | | | 222-321 | RING finger domain |
| human PLCγ-1 | 7A | 13 | 321-454 | PLC catalytic domain |
| | | | 925-1070 | |
| | | | 550-657 | SH2 domain |
| | | | 667-756 | |
| | | | 793-849 | SH3 domain |
| | | | 864-924 | pleckstrin homology domain |
| | | | 1090-1212 | C2 domain |
| mouse PLCγ-1 | 7B | 14 | 208-342 | PLC catalytic domain |
| | | | 822-957 | |
| | | | 436-545 | SH2 domain |
| | | | 556-644 | |
| | | | 684-737 | SH3 domain |
| | | | 751-821 | pleckstrin homology domain |
| | | | 977-1100 | C2 domain |
| human PKCγθ | 8A | 15 | 160-209 | PKC C1 domain |
| | | | 232-281 | |
| | | | 379-634 | kinase catalytic domain |
| | | | 635-701 | PKC C-terminal domain |
| mouse PKCγθ | 8B | 16 | 160-209 | PKC C1 domain |
| | | | 232-281 | |
| | | | 379-634 | kinase catalytic domain |
| | | | 635-701 | PKC C-terminal domain |
| human RasGAP | 9A | 17 | 179-260 | SH2 domain |
| | | | 351-441 | |
| | | | 287-339 | SH3 domain |
| | | | 494-577 | pleckstrin homology domain |
| | | | 590-709 | C2 domain |
| | | | 714-1044 | GTPase-activating domain |
| | | | 690-980 | IQG1 domain |
| mouse RasGAP (partial) | 9B | 18 | 53-105 | SH3 domain |
| | | | 117-207 | SH2 domain |
| | | | 260-343 | pleckstrin homology domain |
| | | | 356-475 | C2 domain |
| | | | 480-810 | GTPase-activating domain |
| | | | 456-746 | IQG1 domain |
| human Tsg101 | 20A | 19 | 23-161 | ubiquitin-conjugating enzyme catalytic domain |
| | | | 222-389 | ATPase domain |
| | | | 243-342 | syntaxin homology domain |
| mouse Tsg101 | 20B | 20 | 23-172 | ubiquitin-conjugating enzyme catalytic domain |
| | | | 223-390 | ATPase domain |
| | | | 244-343 | syntaxin homology domain |

Useful fragments of the present invention can be in an isolated form or as a part of a longer amino acid sequence (e.g., as a component of a fusion protein, and the like). Nucleic acid sequences comprising sequences encoding useful fragments of anergy associated proteins (e.g., nucleic acid sequences encoding any of the protein fragments described above) can be utilized in the methods of the present invention as well.

Fragments of a protein can be produced by any of a variety of methods known to those skilled in the art, e.g., recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. Expression of the mutagenized DNA produces polypeptide fragments. Digestion with “end-nibbling” endonucleases can thus generate DNAs that encode an array of fragments. DNAs that encode fragments of a protein can also be generated, e.g., by random shearing, restriction digestion, chemical synthesis of oligonucleotides, amplification of DNA using the polymerase chain reaction, or a combination of the above-discussed methods.

Fragments can also be chemically synthesized using techniques known in the art, e.g., conventional Merrifield solid phase f-Moc or t-Boc chemistry. For example, peptides of the present invention can be arbitrarily divided into fragments of desired length with no overlap of the fragments, or divided into overlapping fragments of a desired length.

Also useful in the methods of the present invention are variants of the anergy associated proteins or fragments that include “non-essential” amino acid substitutions. Non-essential amino acid substitutions refer to alterations from a wild-type sequence that can be made without abolishing or without substantially altering a biological activity, whereas an “essential” amino acid residue results in such a change.

Auto Ubiquitination Assay

There are at least two types of anergy associated E3 ubiquitin ligases. One type of ligase is referred to as a catalytic (HECT domain) type E3 ligase, which can autoubiquitinate by transferring ubiquitin from the catalytic cysteine (thioester bond) to adjacent ε-amino groups of appropriately positioned lysine residues in the HECT domain or other nearby domains. Another type of E3 ubiquitin ligase is discussed in further detail below. Itch and Aip4 (the human homolog of Itch) are HECT domain-type E3 ligases, and the HECT domain of these ligases is sufficient to cause autoubiquitination. The design of the autoubiquitination assay is based on monitoring autoubiquitination of Itch and/or its human homologue AIP4.

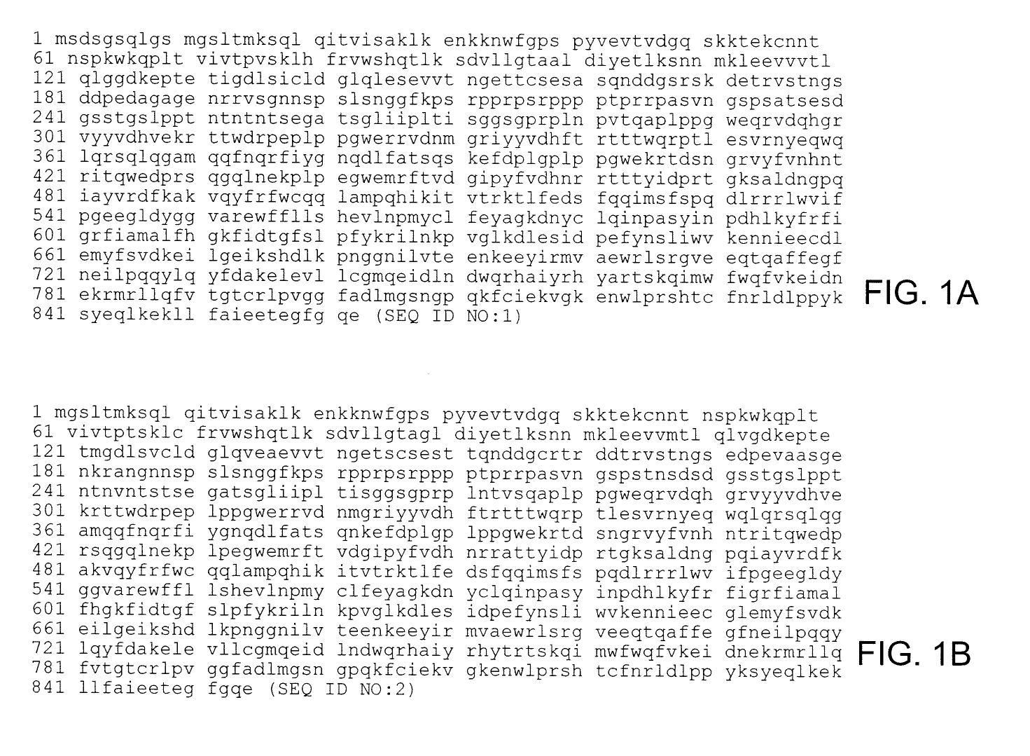

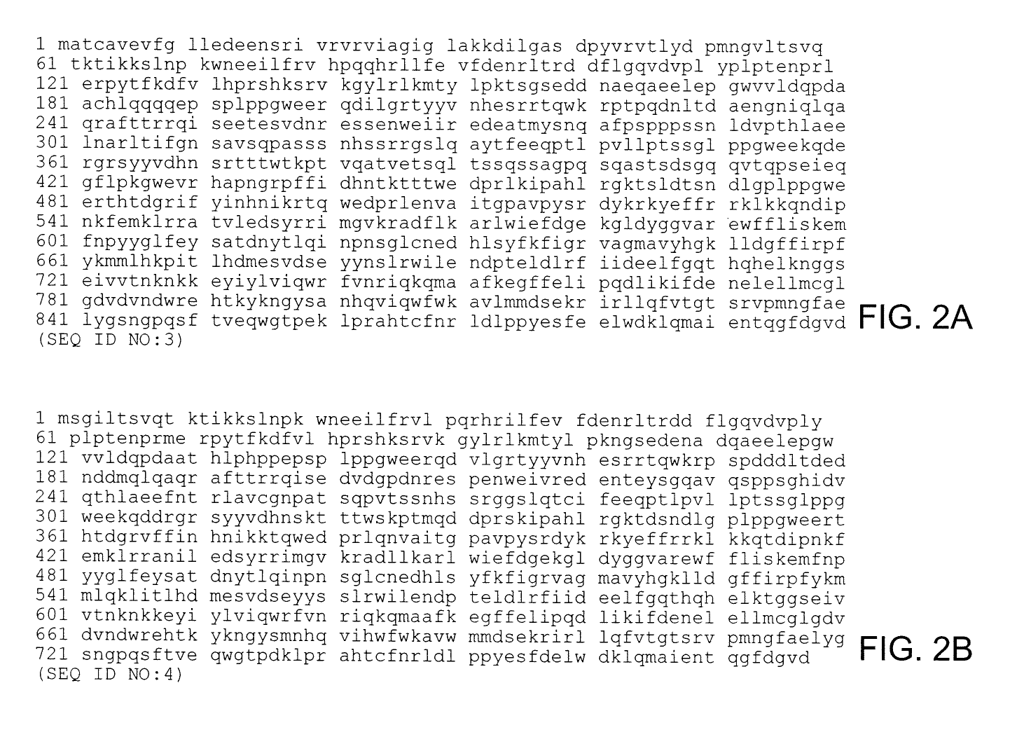

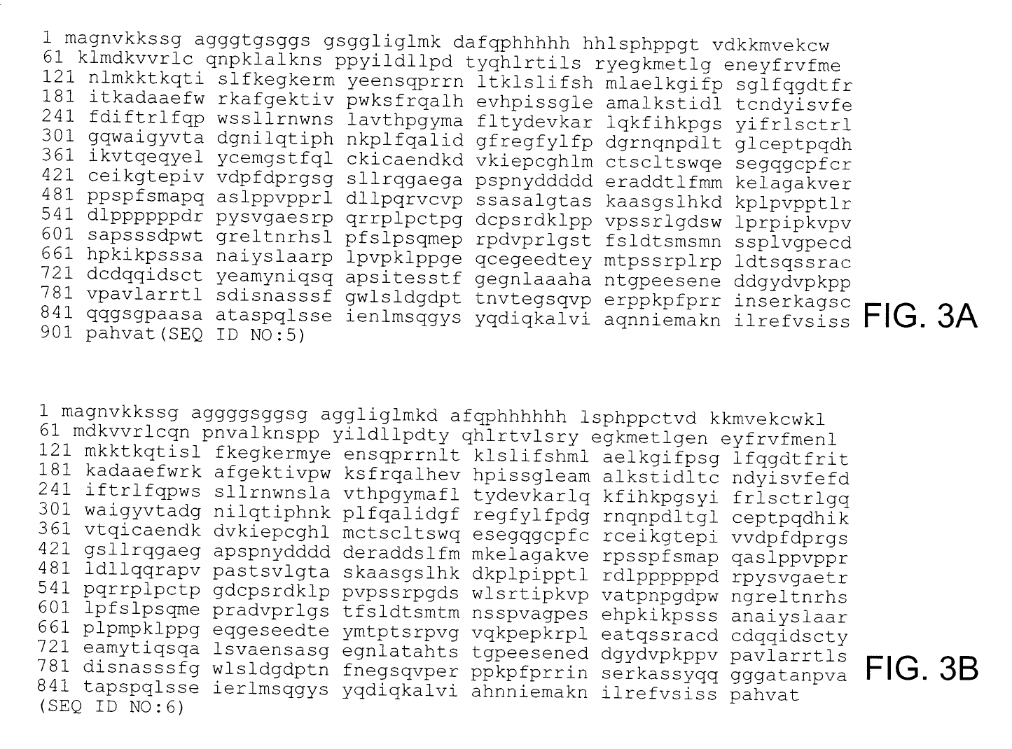

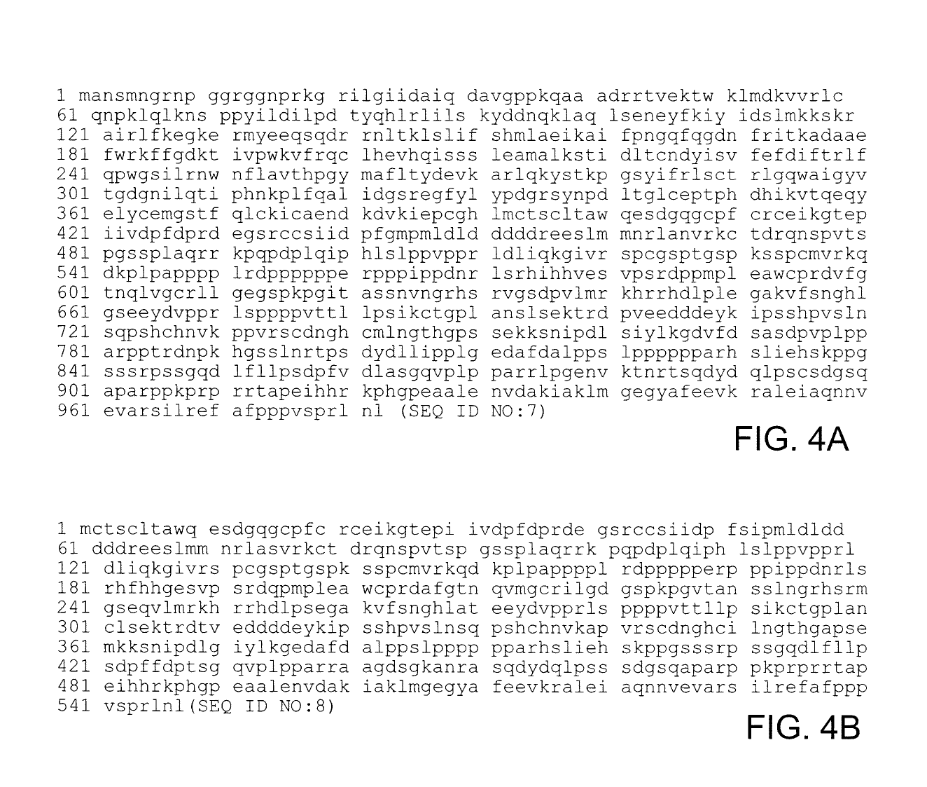

In the assay, Itch or Aip4 proteins are provided. The amino acid sequences of Itch and Aip4 are provided in FIGS. 1B and 1A, respectively. The whole protein (i.e., the entire Itch or AIP4 amino acid sequence) or a fragment thereof can be provided, depending upon the application. In one embodiment, a biologically active fragment of Itch or AIP4 is provided, such as the HECT domains of Itch or AIP4.

The Itch or AIP4 protein or fragment can be provided in an isolated form (e.g., not fused to any other sequence), or as a fusion protein. For example, the sequence can be fused to any other sequence that facilitates isolation and/or purification of the Itch or AIP4 sequence, and/or to another sequence that may be useful in the assay (e.g., a reporter gene). Exemplary sequences useful for isolation/purification include, e.g., hemaglutinin (HA) and glutathione-S-transerfase (GST), among others. Exemplary reporter proteins include, e.g., proteins encoded by lacZ, cat, gus, green fluorescent protein gene, and luciferase gene.

A test compound is provided for screening. A “test compound” can be any chemical compound, for example, a macromolecule (e.g., a polypeptide, a protein complex, or a nucleic acid) or a small molecule (e.g., an amino acid, a nucleotide, an organic or inorganic compound). The test compound can have a formula weight of less than about 10,000 grams per mole, less than 5,000 grams per mole, less than 1,000 grams per mole, or less than about 500 grams per mole. The test compound can be naturally occurring (e.g., an herb or a natural product), synthetic, or can include both natural and synthetic components. Examples of test compounds include peptides, peptidomimetics (e.g., peptoids), amino acids, amino acid analogs, polynucleotides, polynucleotide analogs, nucleotides, nucleotide analogs, and organic or inorganic compounds, e.g., heteroorganic or organometallic compounds.

The Itch or AIP4 protein (or biologically active fragment of either) is then contacted with the test compound. Contacting can be performed in/on any support, e.g., a multiwell plate (e.g., 96-well or 384-well plate), test tube, petri plate, or chip (e.g., a silicon, ceramic, or glass chip). Optionally, the Itch or AIP4 protein or fragment is immobilized in/on the support, e.g., using antibodies, such as an anti-HA antibody (e.g., 12CA5 antibody, i.e., where the protein is fused to an HA sequence) or an antibody raised against the Itch or AIP4 protein or fragment (i.e., an antibody raised against a non-biologically active portion of the protein or fragment). The test compound and protein can optionally be incubated together for a period of time.

A determination is then made as to whether the test compound is capable of binding to and/or preventing autoubiquitination by the Itch or AIP4 protein or fragments thereof. Such a determination can be made using any method known in the art. In one embodiment, whether the test compound is capable of preventing autoubiquitination is determined by adding to the Itch or Aip4 protein a reaction mix containing the enzymes and substrates required by the Itch or Aip4 protein to autoubiquitinate, e.g., purified E1 ubiquitin-activating enzyme, E2 ubiquitin-conjugating enzymes (an example of which is UbCH7), tagged ubiquitin and/or ATP. A discussion of E1, E2, and E3 enzymes can be found in Pickart, Mechanisms Underlying Ubiquitination, Annu. Rev. Biochem. 70, 503-533 (2001), the contents of which is incorporated herein by reference in its entirety. In any of the assays described herein, E1 and/or E2 can be “precharged” with tagged ubiquitin (e.g., wherein E1-ubiquitin and/or E2-ubiquitin is provided). After an incubation period, the reaction can be stopped (e.g., by adding EDTA to the mixture), the support can be washed, and streptavidin-HRP (horseradish peroxidase) can be added to the mixture (i.e., to detect ubiquitin). A substrate for colorimetric detection of the presence of streptavidin-HRP can then be added, and the results can be analyzed. In such an embodiment, the results can be analyzed using an enzyme-linked immunosorbant assay (ELISA) plate reader. In another embodiment, after the reaction mix containing the enzymes and substrates is added to the Itch or Aip4 protein and test compound mix, whether the test compound is capable of preventing autoubiquitination can be determined using SDS-PAGE and immunoblotting techniques.

Test Compounds

The test compounds referred to herein, can be screened individually or in parallel. An example of parallel screening is a high throughput screen of large libraries of chemicals. Such libraries of test compounds can be purchased, e.g., from Chembridge Corp., San Diego, Calif. (e.g., ChemBridge Diverset E). Libraries can be designed to cover a diverse range of compounds. For example, a library can include 500, 1000, 10,000, 50,000, or 100,000 or more unique compounds. Alternatively, prior experimentation and anecdotal evidence can suggest a class or category of compounds of enhanced potential. A library can be designed and synthesized to cover such a class of chemicals.

Rather than purchasing, a library may be generated. Examples of methods for the synthesis of libraries can be found in the literature, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, E. M. Gordon et al., J. Med. Chem. (1994) 37:1385-1401; DeWitt, S. H.; Czarnik, A. W. Acc. Chem. Res. (1996) 29:114; Armstrong, R. W.; Combs, A. P.; Tempest, P. A.; Brown, S. D.; Keating, T. A. Acc. Chem. Res. (1996) 29:123; Ellman, J. A. Acc. Chem. Res. (1996) 29:132; Gordon, E. M.; Gallop, M. A.; Patel, D. V. Acc. Chem. Res. (1996) 29:144; Lowe, G. Chem. Soc. Rev. (1995) 309, Blondelle et al. Trends Anal. Chem. (1995) 14:83; Chen et al. J. Am. Chem. Soc. (1994) 116:2661; U.S. Pat. Nos. 5,359,115, 5,362,899, and 5,288,514; PCT Publication Nos. WO92/10092, WO93/09668, WO91/07087, WO93/20242, WO94/08051).

Libraries of compounds can be prepared according to a variety of methods, some of which are known in the art. For example, to create a library of peptides, a “split-pool” strategy can be implemented in the following way: beads of a functionalized polymeric support are placed in a plurality of reaction vessels; a variety of polymeric supports suitable for solid-phase peptide synthesis are known, and some are commercially available (for examples, see, e.g., M. Bodansky “Principles of Peptide Synthesis”, 2nd edition, Springer-Verlag, Berlin (1993)). To each aliquot of beads is added a solution of a different activated amino acid, and the reactions are allow to proceed to yield a plurality of immobilized amino acids, one in each reaction vessel. The aliquots of derivatized beads are then washed, “pooled” (i.e., recombined), and the pool of beads is again divided, with each aliquot being placed in a separate reaction vessel. Another activated amino acid is then added to each aliquot of beads. The cycle of synthesis is repeated until a desired peptide length is obtained. The amino acid residues added at each synthesis cycle can be randomly selected; alternatively, amino acids can be selected to provide a “biased” library, e.g., a library in which certain portions of the inhibitor are selected non-randomly, e.g., to provide an inhibitor having known structural similarity or homology to a known peptide capable of interacting with an antibody, e.g., an anti-idiotypic antibody antigen binding site. It will be appreciated that a wide variety of peptidic, peptidomimetic, or non-peptidic compounds can be readily generated in this way.

The “split-pool” strategy results in a library of peptides, e g, inhibitors, which can be used to prepare a library of test compounds of the invention. In another illustrative synthesis, a “diversomer library” is created by the method of Hobbs DeWitt et al. (Proc. Natl. Acad. Sci. U.S.A. 90:6909 (1993)). Other synthesis methods, including the “tea-bag” technique of Houghten (see, e.g., Houghten et al., Nature 354:84-86 (1991)) can also be used to synthesize libraries of compounds.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

Libraries of compounds can be screened to determine whether any members of the library have a desired activity, and, if so, to identify the active species. Methods of screening combinatorial libraries are well known in the art and have been described (see, e.g., Gordon et al., J Med. Chem., supra).

Ubiquitin Transfer Assay

The present invention also provides a ubiquitin transfer assay. The assay can be used with catalytic (HECT domain) type E3 ligases or another type of E3 ligases, known as non-catalytic adapter type ligases. Adapter type E3 ligases bridge E2 ubiquitin ligases with their substrates. Adapter-type E3 ligases include Skp1/Cullin/F-box protein (SCF) complexes such as β-TrCP required for IκB degradation; SOCS proteins which downregulate cytokine signalling; and RING-finger proteins (e.g. Cbl, Cbl-b, and GRAIL). In this assay, test compounds are screened for the ability to inhibit ubiquitin transfer from the ligase (or biologically active fragment thereof) onto substrate proteins. For example, PLC-γ1, PKCθ, and RasGap are substrates for the Itch protein (see Example 3, below).

In one embodiment, test compounds are screened for the ability to prevent full-length AIP4/Itch proteins, or fragments thereof, from ubiquitinating and/or binding to full-length or N- or C-terminally deleted fragments of PLC-γ1 or PKCθ. The PLC-γ1 or PKCθ proteins can be either in vitro-translated or expressed in HEK-293 cells. The library screen is performed in a fashion similar to that described for the autoubiquitination screen (above), except that the reaction mix contains not only E1, E2, tagged ubiquitin (e.g., biotin tagged ubiquitin) and/or ATP, but also a substrate capable of being transubiquitinated by the E3 ligase (e.g., PLC-γ1 or PKCθ, e.g., where AIP4 and/or Itch proteins are used) and any other adapters or cofactors that might be needed for efficient transubiquitination.

Other Assays

The invention also includes methods, e.g., for screening (e.g., in a high throughput manner) test compounds to identify agents capable of binding to anergy associated E3 ubiquitin ligases and/or ligase substrates, inhibiting protein-protein interactions between E3 ubiquitin ligases and ligase substrates, and inhibiting production (e.g., transcription) of E3 ubiquitin ligases.

In one assay for identifying agents capable of inhibiting protein-protein interactions, a first compound is provided. The first compound is an E3 ubiquitin ligase or a biologically active fragment thereof, or the first compound is a ligase substrate or a biologically active derivative thereof. A second compound is provided which is different from the first compound and which is labeled. The second compound is an E3 ubiquitin ligase or a biologically active fragment thereof, or the second compound is a ligase substrate or a biologically active derivative thereof. A test compound is provided. The first compound, second compound and test compound are contacted with each other. The amount of label bound to the first compound is determined. A reduction in protein-protein interaction between the first compound and the second compound as assessed by label bound is indicative of the usefulness of the agent in inhibiting protein-protein interactions between anergy associated E3 ubiquitin ligases and ligase substrates. The reduction can be assessed relative to the same reaction without addition of the candidate agent.

In certain embodiments, the first compound is attached to a solid support. Solid supports include, e.g., resins, e.g., agarose and a multiwell plate. In certain embodiments, the method includes a washing step after the contacting step, so as to separate bound and unbound label.

By high-throughput screening is meant that the method can be used to screen a large number of candidate agents easily and quickly. In some embodiments, a plurality of candidate compounds is contacted with the first compound and second compound. The different candidate compounds can be contacted with the other compounds in groups or separately. In one embodiment, each of the candidate compounds is contacted with both the first compound and the second compound in separate wells. For example, the method can screen libraries of potential agents. The libraries can be in a form compatible with screening in multiwell plates, e.g., 96-well plates. The assay is particularly useful for automated execution in a multiwell format in which many of the steps are controlled by computer and carried out by robotic equipment, as are all assays described herein. The libraries can also be used in other formats, e.g., synthetic chemical libraries affixed to a solid support and available for release into microdroplets.

In certain embodiments, the first compound is an E3 ubiquitin ligase or a biologically active derivative thereof, and the second compound is an E3 ubiquitin ligase substrate or a biologically active derivative thereof. In other embodiments, the first compound is E3 ubiquitin ligase substrate or a biologically active derivative thereof, and the second compound is E3 ubiquitin ligase or a biologically active derivative thereof. The second compound can be labeled with any label that will allow its detection, e.g., a radiolabel, a fluorescent agent, biotin, a peptide tag, or an enzyme fragment. In certain embodiments, the second compound is radiolabeled, e.g., with ¹²⁵I or ³H.

In certain embodiments, the enzymatic activity of an enzyme chemically conjugated to, or expressed as a fusion protein with, the first or second compound, is used to detect bound protein. A binding assay in which a standard immunological method is used to detect bound protein is also included. Methods based on surface plasmon resonance, as, e.g., in the BIAcore biosensor (Pharmacia Biosensor, Uppsala, Sweden) or evanescent wave excitation of fluorescence can be used to measure recruitment of, e.g., E3 ubiquitin ligase substrate (or fluorescently labeled ligase substrate) to a surface on which E3 ubiquitin ligase is immobilized. In certain other embodiments, the interaction of E3 ubiquitin ligase and substrate is detected by fluorescence resonance energy transfer (FRET) between a donor fluorophore covalently linked to E3 ubiquitin ligase substrate (e.g., a fluorescent group chemically conjugated to E3 ubiquitin ligase substrate, or a variant of green fluorescent protein (GFP) expressed as an E3 ubiquitin ligase substrate-GFP chimeric protein) and an acceptor fluorophore covalently linked to an E3 ubiquitin ligase, where there is suitable overlap of the donor emission spectrum and the acceptor excitation spectrum to give efficient nonradiative energy transfer when the fluorophores are brought into close proximity through the protein-protein interaction of E3 ubiquitin ligase and its substrate.

In certain embodiments, the protein-protein interaction is detected by reconstituting domains of an enzyme, e.g., β-galactosidase (e.g., a two-hybrid system) (see, e.g., Rossi et al, Proc. Natl. Acad. Sci. USA 94:8405-8410 (1997)). The detection method used is appropriate for the particular enzymatic reaction, e.g., by chemiluminescence with Galacton Plus substrate from the Galacto-Light Plus assay kit (Tropix, Bedford, Mass.) or by fluorescence with fluorescein di-β-D-galactopyranoside (Molecular Probes, Eugene, Oreg.) for β-galactosidase. Competition of the protein-protein interaction by the candidate agents is evident in a reduction of the measured enzyme activity. This assay can be performed with proteins in vitro or in vivo. An advantage of this embodiment in vivo is that only compounds sufficiently permeable through the membrane of living cells will be scored positive, and thus agents most likely to reach effective concentrations within cells when administered therapeutically can be identified. Measurement of reconstituted β-galactosidase activity in living cells has been demonstrated with fluorescein di-β-D-galactopyranoside (Molecular Probes, Eugene, Oreg.) as substrate. See Rossi et al., Proc. Natl. Acad. Sci. USA 94:8405-8410 (1997).

In certain embodiments, the protein-protein interaction is assessed by fluorescence ratio imaging (Bacskai et al, Science 260:222-226 (1993)) of suitable chimeric constructs of E3 ubiquitin ligase and substrates in cells, or by variants of the two-hybrid assay (Fearon et al, Proc Natl Acad Sci USA 89:7958-7962 (1992); Takacs et al, Proc Natl Acad Sci USA 90:10375-10379 (1993); Vidal et al, Proc Natl Acad Sci USA 93:10315-10320 (1996); Vidal et al, Proc Natl Acad Sci USA 93:10321-10326 (1996)) employing suitable constructs of E3 ubiquitin ligase and substrates. The fluorescence ratio imaging and variant two-hybrid systems can be tailored for a high throughput assay to detect compounds that inhibit the protein-protein interaction.

Other methods for identifying agents include various cell-based methods for identifying compounds that bind E3 ubiquitin ligases, or homologs or orthologs thereof, such as the conventional two-hybrid assays of protein/protein interactions (see e.g., Chien et al., Proc. Natl. Acad. Sci. USA, 88:9578, 1991; Fields et al., U.S. Pat. No. 5,283,173; Fields and Song, Nature, 340:245, 1989; Le Douarin et al., Nucleic Acids Research, 23:876, 1995; Vidal et al., Proc. Natl. Acad. Sci. USA, 93:10315-10320, 1996; and White, Proc. Natl. Acad. Sci. USA, 93:10001-10003, 1996). Generally, the two-hybrid methods involve reconstitution of two separable domains of a transcription factor in a cell. One fusion protein contains the E3 ubiquitin ligase (or homolog or ortholog thereof) fused to either a transactivator domain or DNA binding domain of a transcription factor (e.g., of Ga14). The other fusion protein contains an E3 ubiquitin ligase substrate fused to either the DNA binding domain or a transactivator domain of a transcription factor. Once brought together in a single cell (e.g., a yeast cell or mammalian cell), one of the fusion proteins contains the transactivator domain and the other fusion protein contains the DNA binding domain. Therefore, binding of the E3 ubiquitin ligase to the substrate (i.e., in the absence of an inhibitor) reconstitutes the transcription factor. Reconstitution of the transcription factor can be detected by detecting expression of a gene (i.e., a reporter gene) that is operably linked to a DNA sequence that is bound by the DNA binding domain of the transcription factor. Kits for practicing various two-hybrid methods are commercially available (e.g., from Clontech; Palo Alto, Calif.).

In one assay for identifying agents capable of binding to E3 ubiquitin ligase or ligase substrate, binding of a test compound to a target protein is detected using capillary electrophoresis. Briefly, test compounds (e.g., small molecules) that bind to the target protein cause a change in the electrophoretic mobility of the target protein during capillary electrophoresis. Suitable capillary electrophoresis methods are known in the art (see, e.g., Freitag, J. Chromatography B, Biomedical Sciences & Applications: 722(1-2):279-301, Feb. 5, 1999; Chu and Cheng, Cellular & Molecular Life Sciences: 54(7):663-83, July 1998; Thormann et al., Forensic Science International: 92(2-3): 157-83, Apr. 5, 1998; Rippel et al., Electrophoresis: 18(12-13): 2175-83, November 1997; Hage and Tweed, J. Chromatography. B, Biomedical Sciences & Applications: 699(1-2):499-525, Oct. 10, 1997; Mitchelson et al., Trends In Biotechnology: 15(11):448-58, November 1997; Jenkins and Guerin J. Chromatography B. Biomedical Applications: 682(1):23-34, Jun. 28, 1996; and Chen and Gallo, Electrophoresis: 19(16-17):2861-9, November 1998.

In one assay for identifying agents capable of inhibiting production (e.g., transcription) of E3 ubiquitin ligases, a cell (e.g., an immune cell, e.g., a T- or a B-cell or cell line) is provided and contacted with a test agent. Whether the test agent modulates, e.g., inhibits, transcription of at least one E3 ubiquitin ligase (i.e., Itch, Cbl-b, Cbl, Cbl-3, Grail, Nedd4, and/or Aip4) or the ubiquitin receptor Tsg101 gene is then determined. A change, e.g., a decrease, in the level of transcription of the E3 ubiquitin ligase, and/or Tsg101, is indicative of the usefulness of the compound as a compound capable of modulating anergy. Transcription can be measured using any art known method, e.g., by measuring mRNA levels of one or more of the proteins.

In another assay for identifying agents capable of inhibiting production (e.g., transcription and/or translation) of anergy associated E3 ubiquitin ligases, a reporter gene coupled to the promoter of the anergy associated-gene is utilized to monitor the expression of the E3 ubiquitin ligase in the presence of an anergic state-inducing agent (e.g., ionomycin) and/or a test compound. To construct the reporter, the promoter of the selected gene (e.g., genes encoding one or more of Itch, Cbl-b, Cbl, Cbl-3, Grail, Nedd4, and/or Aip4) can be operably linked to a reporter gene, e.g., without utilizing the reading frame of the selected gene. Table 2, below, lists Genebank accession numbers for large genomic fragments of Itch, Cbl-b, Cbl, Cbl-3, Grail, Nedd4, and Aip4 together with the nucleotide range of the promoter within that fragment.

TABLE 2
Exemplary Promoters Regions

| promoter for: | Nucleotide accession # | subsequence | |
|---|---|---|---|
| human Aip4 | NT_028392.4 | 3112852 | 3117851 |
| mouse itch | NT_039210.1 | 3788654 | 3793653 |
| human cbl-b | NT_005612.13 | 11986983 | 11991982 |
| mouse cbl-b | NT_039624.1 | 49100606 | 49105605 |
| human cbl | NT_033899.5 | 22615668 | 22620667 |
| mouse cbl | NT_039473.1 | 3658578 | 3663577 |
| human cbl-3 | NT_011109.15 | 17544366 | 17549365 |
| mouse cbl-3 | NT_039400.1 | 1087708 | 1092707 |
| human Grail | NT_011651.13 | 29202698 | 29207697 |
| mouse Grail | NT_039716.1 | 4233285 | 4238284 |
| human Nedd4 | NT_010194.15 | 27075447 | 27080446 |
| mouse Nedd4 | NT_039474 | 19020597 | 19025596 |

The nucleic acid construction can be transformed into cultured cells, e.g., T cells, by a transfection protocol or lipofection to generate reporter cells. The reporter gene can be, e.g., green fluorescent protein, β-galactosidase, alkaline phosphatase, β-lactamase, luciferase, or chloramphenicol acetyltransferase. The nucleic acid construction can be maintained on an episome or inserted into a chromosome, for example using targeted homologous recombination as described in Chappel, U.S. Pat. No. 5,272,071 and WO 91/06667.

In an embodiment utilizing green fluorescent protein (GFP) or enhanced GFP (eGFP) (Clontech, Palo Alto, Calif.) the reporter cells are grown in microtiter plates wherein each well is contacted with a unique agent to be tested. Following desired treatment duration, e.g., 5 hours, 10 hours, 20 hours, 40 hours, or 80 hours, the microtiter plate is scanned under a microscope using UV lamp emitting light at 488 nm. A CCD camera and filters set to detect light at 509 nm is used to monitor the fluorescence of eGFP, the detected fluorescence being proportional to the amount of reporter produced.

In an embodiment utilizing β-galactosidase, a substrate that produces a luminescent product in a reaction catalyzed by β-galactosidase is used. Again, reporter cells are grown in microtiter plates and contacted with compounds for testing. Following treatment, cells are lysed in the well using a detergent buffer and exposed to the substrate. Lysis and substrate addition can be achieved in a single step by adding a buffer which contains a 1:40 dilution of Galacton-Star™ substrate (3-chloro-5-(4-methoxyspiro{1,2-dioxetane-3,2′-(4′chloro)-tricyclo-[3.3.1.1^{3,7}]decan}-4-yl)phenyl-B-D-galactopyranoside; Tropix, Inc., Cat.# GS100), a 1:5 dilution of Sapphire II™ luminescence signal enhancer (Tropix, Inc., Cat.#LAX250), 0.03% sodium deoxycholic acid, 0.053% CTAB, 250 mM NaCl, 300 mM HEPES, pH 7.5). The cells are incubated in the mixture at room temperature for approximately 2 hours prior to quantitation. β-galactosidase activity is monitored by the chemiluminescence produced by the product of β-galactosidase hydrolysis of the Galacton-Star™ substrate. A microplate reader fitted with a sensor can be used to quantitate the light signal. Standard software, for example, Spotfire Pro version 4.0 data analysis software, can be utilized to analyze the results. The mean chemiluminescent signal for untreated cells is determined. Compounds that exhibit a signal at least 2.5 standard deviations above the mean can be candidates for further analysis and testing. Similarly, for alkaline phosphatase, β-lactamase, and luciferase, substrates are available which are fluorescent when converted to product by enzyme.

Secondary Assays

Once a test compound is identified using one of the above-described assays, the test compound can optionally be further tested in a secondary assay. Such secondary assays can be used, e.g., to analyze the specificity of the isolated test compound and/or to confirm the anergy-modulating activity of the test compound. The secondary assay can involve, e.g., performing/repeating any assay described above, or an assay described below.

For example, with regard to specificity, ubiquitination assays similar to those described above can be performed, using E1 alone or E1+E2 alone, in the presence or absence of the test compounds, in order to determine if the test compounds block thioester bond formation or ubiquitin transfer in general. The resulting proteins can be analyzed by resolving the proteins on polyacrylamide gels under reducing or non-reducing conditions (the thioester bond is labile under reducing conditions whereas the isopeptide bond is not). As another example, a test compound found to display activity (e.g., binding activity) against one type of anergy associated E3 ubiquitin ligase and/or ligase substrate can be tested in a secondary assay against one or more of the other E3 ubiquitin ligases or ligase substrates.

With regard to confirmatory secondary assays, co-transfection experiments can be performed in a cell-based assay. For example, cells (e.g., HEK 293 cells) can be cotransfected with Itch, HA-ubiquitin and PLC-γ1 or PKCθ, and the ability of the test compound to inhibit substrate ubiquitination and degradation can be examined. Controls can include using NFκB p105 or IκBα and β-TrCP, or E6AP, E6 and p53. If test compounds are effective in such a cell-based assay, they are also likely to be cell-permeant.

Alternatively or in addition, whether the test compound can modulate anergy in a cell-based assay can be determined. Test compounds isolated using the methods described herein can be assayed to determine whether they are capable of inhibiting PLC-γ1 and PKCθ degradation, rescuing Ca²⁺ mobilization, and/or rescuing proliferation in T cells, after they have been exposed to anergy-inducing stimuli (e.g., ionomycin). Cells can be treated with ionomycin for 16 h, then incubated with the test compound during the step of restimulation through the TCR. Such assays can be carried out as described in the Example section, below.

Medicinal Chemistry

Once a compound (or agent) of interest has been identified, standard principles of medicinal chemistry can be used to produce derivatives of the compound. Derivatives can be screened for improved pharmacological properties, for example, efficacy, pharmacokinetics, stability, solubility, and clearance. The moieties responsible for a compound's activity in the assays described above can be delineated by examination of structure-activity relationships (SAR) as is commonly practiced in the art. A person of ordinary skill in pharmaceutical chemistry could modify moieties on a lead compound and measure the effects of the modification on the efficacy of the compound to thereby produce derivatives with increased potency. For an example, see Nagarajan et al. (1988) J. Antibiot. 41: 1430-8. Furthermore, if the biochemical target of the compound (or agent) is known or determined, the structure of the target and the compound can inform the design and optimization of derivatives. Molecular modeling software is commercially available (e.g., Molecular Simulations, Inc.) for this purpose.

Pharmaceutical Compositions

The compounds, nucleic acids, and polypeptides, fragments thereof, as well as antibodies, e.g., anti-E3 ubiquitin ligase polypeptide antibodies other molecules and agents of the invention (also referred to herein as “active compounds”) can be incorporated into pharmaceutical compositions. Such compositions typically include the nucleic acid molecule, protein, or antibody and a pharmaceutically acceptable carrier. A “pharmaceutically acceptable carrier” can include solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be achieved by including an agent which delays absorption, e.g., aluminum monostearate and gelatin in the composition.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

Therapeutic compositions can be administered with medicinal devices known in the art. For example, in a preferred embodiment, a therapeutic composition of the invention can be administered with a needleless hypodermic injection device, such as the devices disclosed in U.S. Pat. No. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the compounds of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. While compounds that exhibit toxic side effects may be used, care can be taken to design a delivery system that targets such compounds to the site of interest.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

For the anergy modulating agents described herein, an effective amount, e.g. of a protein or polypeptide (i.e., an effective dosage), can range from about 0.001 to 30 mg/kg body weight, e.g. about 0.01 to 25 mg/kg body weight, e.g. about 0.1 to 20 mg/kg body weight. A protein or polypeptide can be administered one time per week for between about 1 to 10 weeks, e.g. between 2 to 8 weeks, about 3 to 7 weeks, or for about 4, 5, or 6 weeks. The skilled artisan will appreciate that certain factors influence the dosage and timing required to effectively treat a patient, including but not limited to the type of patient to be treated, the severity of the disease or disorder, previous treatments, the general health and/or age of the patient, and other diseases present. Moreover, treatment of a patient with a therapeutically effective amount of a protein, polypeptide, antibody, or other compound can include a single treatment or, preferably, can include a series of treatments.

For antibodies, a useful dosage is 0.1 mg/kg of body weight (generally 10 mg/kg to 20 mg/kg). Generally, partially human antibodies and fully human antibodies have a longer half-life within the human body than other antibodies. Accordingly, lower dosages and less frequent administration are possible. Modifications such as lipidation can be used to stabilize antibodies and to enhance uptake and tissue penetration. A method for lipidation of antibodies is described by Cruikshank et al. ((1997) J. Acquired Immune Deficiency Syndromes and Human Retrovirology 14:193).

If the agent is a small molecule, exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Anergy Modulating Compounds and Modulation of Anergy

The invention provides methods for modulating, e.g., inhibiting (e.g., limiting, preventing or reducing) anergy. Compounds capable of modulating anergy can be used, e.g., for treating and/or preventing disorders, such as cancers, immune cell disorders, e.g., T cell disorders, and infectious disorders. The compounds can be useful in boosting the immune response to tumors, and may be particularly useful for eliminating surviving tumor cells after chemotherapy.

A compound capable of inhibiting anergy associated protein production, binding, and/or activity can be a chemical, e.g., a small molecule (e.g., a chemical agent having a molecular weight of less than 2500 Da, e.g., from at least about 100 Da to about 2000 Da (e.g., between about 100 to about 2000 Da, about 100 to about 1750 Da, about 100 to about 1500 Da, about 100 to about 1250 Da, about 100 to about 1000 Da, about 100 to about 750 Da, about 100 to about 500 Da, about 200 to about 1500, about 500 to about 1000, about 300 to about 1000 Da, or about 100 to about 250 Da), e.g., a small organic molecule, e.g., a product of a combinatorial library.

In other embodiments, the compound is a polypeptide (e.g., an antibody such as an intrabody), a peptide, a peptide fragment, a peptidomimetic, an antisense oligonucleotide, and/or a ribozyme. Compounds may be isolated from a natural products library, e.g., microbial broths or extracts from diverse stains of bacteria, fungi, and actinomycetes (MDS Panlabs, Bothell, Wash.); a combinatorial chemical library, e.g., an Optiverse™ Screening Library (MDS Panlabs, Bothell, Wash.); an encoded combinatorial chemical library synthesized using ECLiPS™ technology (Pharmacopeia, Princeton, N.J.); and/or another organical chemical, combinatorial chemical, or natural products library assembled according to methods known to those skilled in the art and e.g., formatted for high-throughput screening.

With regard to inhibiting anergy associated protein production, the compound can be, for example, an antisense nucleic acid effective to inhibit expression of an E3 ubiquitin ligase, i.e., Itch, Cbl-b, Cbl, Cbl-3, Grail, Nedd4, and/or Aip4. The antisense nucleic acid can include a nucleotide sequence complementary to an entire anergy associated E3 ubiquitin ligase RNA or only a portion of the RNA. On one hand, the antisense nucleic acid needs to be long enough to hybridize effectively with the RNA. Therefore, the minimum length is approximately 10, 11, 12, 13, 14, or 15 nucleotides. On the other hand, as length increases beyond about 150 nucleotides, effectiveness at inhibiting translation increases only marginally, while difficulty in introducing the antisense nucleic acid into a target area (e.g., target cells) may increase significantly. In view of these considerations, a preferred length for the antisense nucleic acid is from about 15 to about 150 nucleotides, e.g., 20, 25, 30, 35, 40, 45, 50, 60, 70, or 80 nucleotides. The antisense nucleic acid can be complementary to a coding region of the mRNA or a 5′ or 3′ non-coding region of the mRNA (or both). One approach is to design the antisense nucleic acid to be complementary to a region on both sides of the translation start site of the mRNA.

The antisense nucleic acid can be chemically synthesized, e.g., using a commercial nucleic acid synthesizer according to the vendor's instructions. Alternatively, the antisense nucleic acids can be produced using recombinant DNA techniques. An antisense nucleic acid can incorporate only naturally occurring nucleotides. Alternatively, it can incorporate variously modified nucleotides or nucleotide analogs to increase its in vivo half-life or to increase the stability of the duplex formed between the antisense molecule and its target RNA. Examples of nucleotide analogs include phosphorothioate derivatives and acridine-substituted nucleotides. Given the description of the targets and sequences, the design and production of suitable antisense molecules is within ordinary skill in the art. For guidance concerning antisense nucleic acids, see, e.g., Goodchild, “Inhibition of Gene Expression by Oligonucleotides,” in Topics in Molecular and Structural Biology, Vol. 12: Oligodeoxynucleotides (Cohen, ed.), MacMillan Press, London, pp. 53-77.

Delivery of antisense oligonucleotides can be accomplished by any method known to those of skill in the art. For example, delivery of antisense oligonucleotides for cell culture and/or ex vivo work can be performed by standard methods such as the liposome method or simply by addition of membrane-permeable oligonucleotides. To resist nuclease degradation, chemical modifications such as phosphorothionate backbones can be incorporated into the molecule.

Delivery of antisense oligonucleotides for in vivo applications can be accomplished, for example, via local injection of the antisense oligonucleotides at a selected site. This method has previously been demonstrated for psoriasis growth inhibition and for cytomegalovirus inhibition. See, for example, Wraight et al., (2001). Pharmacol Ther. Apr; 90(1):89-104.; Anderson, et al., (1996) Antimicrob Agents Chemother 40: 2004-2011; and Crooke et al., J Pharmacol Exp Ther 277: 923-937.

Similarly, the present invention anticipates that RNA interference (RNAi) techniques could be used in addition or as an alternative to, the use of antisense techniques. For example, small interfering RNA (siRNA) duplexes directed against Itch, Cbl-b, Cbl, Cbl-3, Grail, Nedd4, and Aip4 could be synthesized and used to prevent expression of the encoded protein(s).

As another example, Itch, Cbl-b, Cbl, Cbl-3, Grail, Nedd4, and/or Aip4 activity can be inhibited using an Itch, Cbl-b, Cbl, Cbl-3, Grail, Nedd4, and/or Aip4 polypeptide binding molecule such as an antibody, e.g., an anti-Itch, Cbl-b, Cbl, Cbl-3, Grail, Nedd4, and/or Aip4 polypeptide antibody, or an Itch, Cbl-b, Cbl, Cbl-3, Grail, Nedd4, and/or Aip4 polypeptide-binding fragment thereof. The antibody can be a polyclonal or a monoclonal antibody. Alternatively or in addition, the antibody can be produced recombinantly, e.g., produced by phage display or by combinatorial methods as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982.

As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Each VH and VL is composed of three CDR's and four FRs, arranged from amino-terminus to carboxy-terminus in the following order: FR1, CDR1, FR2, CDR2, FR3, CDR3, FR4.

An anti-E3 ubiquitin ligase (i.e., Itch, Cbl-b, Cbl, Cbl-3, Grail, Nedd4, and/or Aip4) polypeptide antibody can further include a heavy and light chain constant region, to thereby form a heavy and light immunoglobulin chain, respectively. The antibody can be a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2, and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

A “E3 ubiquitin ligase polypeptide-binding fragment” of an antibody refers to one or more fragments of a full-length antibody that retain the ability to specifically bind to an E3 ubiquitin ligase polypeptide or a portion thereof. “Specifically binds” means that an antibody or ligand binds to a particular target to the substantial exclusion of other substances. Examples of polypeptide binding fragments of an anti-E3 ubiquitin ligase polypeptide antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are encoded by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “E3 ubiquitin ligase polypeptide-binding fragment” of an antibody. These antibody fragments can be obtained using conventional techniques known to those with skill in the art.

The anti-E3 ubiquitin ligase polypeptide antibody can be a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel, donkey, porcine, or fowl antibody.

An anti-E3 ubiquitin ligase polypeptide antibody can be one in which the variable region, or a portion thereof, e.g., the CDRs, are generated in a non-human organism, e.g., a rat or mouse. The anti-E3 ubiquitin ligase polypeptide antibody can also be, for example, chimeric, CDR-grafted, or humanized antibodies. The anti-E3 ubiquitin ligase polypeptide antibody can also be generated in a non-human organism, e.g., a rat or mouse, and then modified, e.g., in the variable framework or constant region, to decrease antigenicity in a human.

Treatment of Cancer

Compounds described herein can have therapeutic utilities. For example, the compounds can be administered to cells in culture, e.g. in vitro or ex vivo, or in a patient, e.g., in vivo, to treat and/or prevent disorders, such as cancers, immune cell disorders, e.g., T cell disorders, and infectious disorders. In particular, compounds capable of inhibiting E3 ligase activity are expected to prevent T cells from becoming tolerant to the presence of a tumor (or individual tumor cells) in the body.

As used herein, the terms “cancer”, “hyperproliferative”, “malignant”, and “neoplastic” are used interchangeably, and refer to those cells an abnormal state or condition characterized by rapid proliferation or neoplasm. The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

The common medicinal meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be benign, premalignant or malignant.

The subject method can be useful in treating malignancies of the various organ systems, such as those affecting lung, breast, lymphoid, gastrointestinal (e.g., colon), and genitourinary tract (e.g., prostate), pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Exemplary solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The term “carcinoma” is recognized by those skilled in the art and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is recognized by those skilled in the art and refers to malignant tumors of mesenchymal derivation.

The compounds can also be used in treatments for inhibiting the proliferation of hyperplastic/neoplastic cells of hematopoietic origin, e.g., arising from myeloid, lymphoid or erythroid lineages, or precursor cells thereof. For instance, the present invention contemplates the treatment of various myeloid disorders including, but not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97). Lymphoid malignancies which may be treated by the subject method include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas contemplated by the treatment methods of the present invention include, but are not limited to, non-Hodgkin's lymphoma and variants thereof, peripheral T-cell lymphomas, adult T-cell leukemia/lymphoma (ATL), cutaneous T-cell lymphoma (CTCL), large granular lymphocytic leukemia (LGF) and Hodgkin's disease.

As used herein, the terms “leukemia” or “leukemic cancer” refers to all cancers or neoplasias of the hematopoietic and immune systems (blood and lymphatic system). These terms refer to a progressive, malignant disease of the blood-forming organs, marked by distorted proliferation and development of leukocytes and their precursors in the blood and bone marrow. The acute and chronic leukemias, together with the other types of tumors of the blood, bone marrow cells (myelomas), and lymph tissue (lymphomas), cause about 10% of all cancer deaths and about 50% of all cancer deaths in children and adults less than 30 years old. Chronic myelogenous leukemia (CML), also known as chronic granulocytic leukemia (CGL), is a neoplastic disorder of the hematopoietic stem cell.

Combination Therapy

In one embodiment, the compositions of the invention, e.g., the pharmaceutical compositions, are administered in combination therapy, i.e., combined with other agents, e.g., therapeutic agents, that are useful for treating disorders, such as cancer or T cell-mediated disorders. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment. For example, the combination therapy can include a composition of the present invention coformulated with, and/or coadministered with, one or more additional therapeutic agents, e.g., one or more anti-cancer agents, cytotoxic or cytostatic agents and/or immunosuppressants. For example, the agents of the invention or antibody binding fragments thereof may be coformulated with, and/or coadministered with, one or more additional antibodies that bind other targets (e.g., antibodies that bind other cytokines or that bind cell surface molecules), and/or one or more cytokines. Furthermore, one or more antibodies of the invention may be used in combination with two or more of the foregoing therapeutic agents. Such combination therapies may advantageously utilize lower dosages of the administered therapeutic agents, thus avoiding possible toxicities or complications associated with the various monotherapies.

The terms “cytotoxic agent” and “cytostatic agent” and “anti-tumor agent” are used interchangeably herein and refer to agents that have the property of inhibiting the growth or proliferation (e.g., a cytostatic agent), or inducing the killing, of hyperproliferative cells, e.g., an aberrant cancer cell or a T cell. In cancer therapeutic embodiments, the term “cytotoxic agent” is used interchangeably with the terms “anti-cancer” or “anti-tumor” to mean an agent, which inhibits the development or progression of a neoplasm, particularly a solid tumor, a soft tissue tumor, or a metastatic lesion.

Nonlimiting examples of anti-cancer agents include, e.g., antimicrotubule agents, topoisomerase inhibitors, antimetabolites, mitotic inhibitors, alkylating agents, intercalating agents, agents capable of interfering with a signal transduction pathway, agents that promotes apoptosis and radiation. Examples of the particular classes of anti-cancer agents are provided in detail as follows: antitubulin/antimicrotubule, e.g., paclitaxel, vincristine, vinblastine, vindesine, vinorelbin, taxotere; topoisomerase I inhibitors, e.g., topotecan, camptothecin, doxorubicin, etoposide, mitoxantrone, daunorubicin, idarubicin, teniposide, amsacrine, epirubicin, merbarone, piroxantrone hydrochloride; antimetabolites, e.g., 5-fluorouracil (5-FU), methotrexate, 6-mercaptopurine, 6-thioguanine, fludarabine phosphate, cytarabine/Ara-C, trimetrexate, gemcitabine, acivicin, alanosine, pyrazofurin, N-Phosphoracetyl-L-Asparate=PALA, pentostatin, 5-azacitidine, 5-Aza 2′-deoxycytidine, ara-A, cladribine, 5-fluorouridine, FUDR, tiazofurin, N-[5-[N-(3,4-dihydro-2-methyl-4-oxoquinazolin-6-ylmethyl)-N-methylamino]-2-thenoyl]-L-glutamic acid; alkylating agents, e.g., cisplatin, carboplatin, mitomycin C, BCNU=Carmustine, melphalan, thiotepa, busulfan, chlorambucil, plicamycin, dacarbazine, ifosfamide phosphate, cyclophosphamide, nitrogen mustard, uracil mustard, pipobroman, 4-ipomeanol; agents acting via other mechanisms of action, e.g., dihydrolenperone, spiromustine, and desipeptide; biological response modifiers, e.g., to enhance anti-tumor responses, such as interferon; apoptotic agents, such as actinomycin D; and anti-hormones, for example anti-estrogens such as tamoxifen or, for example antiandrogens such as 4′-cyano-3-(4-fluorophenylsulphonyl)-2-hydroxy-2-methyl-3′-(trifluoromethyl) propionanilide.

A particular combination of cytotoxic agents can be used depending on the condition to be treated. For example, when treating leukemias, in addition to radiation, the following drugs, usually in combinations with each other, are often used: vincristine, prednisone, methotrexate, mercaptopurine, cyclophosphamide, and cytarabine. In chronic leukemia, for example, busulfan, melphalan, and chlorambucil can be used in combination. All of the conventional anti-cancer drugs are highly toxic and tend to make patients quite ill while undergoing treatment. Vigorous therapy is based on the premise that unless every leukemic cell is destroyed, the residual cells will multiply and cause a relapse.

Another aspect of the present invention accordingly relates to kits for carrying out the combined administration of the agents with other therapeutic compounds. In one embodiment, the kit comprises an agent formulated in a pharmaceutical carrier, and at least one cytotoxic agent, formulated as appropriate, in one or more separate pharmaceutical preparations.

Nucleic Acids, Vectors and Host Cells

Another aspect of the invention pertains to isolated nucleic acid, vector and host cell compositions that can be used for expression of the anergy associated nucleic acids of the invention.

Nucleic acids useful in the present invention (e.g., nucleic acids encoding anergy associated E3 ubiquitin ligases and/or ligase substrates) can be chosen for having codons, which are preferred, or non preferred, for a particular expression system (e.g., the nucleic acid can be one in which at least one codon, at preferably at least 10%, or 20% of the codons has been altered such that the sequence is optimized for expression in E. coli, yeast, human, insect, or CHO cells).

In one embodiment, the nucleic acid differs (e.g., differs by substitution, insertion, or deletion) from that of the sequences provided, e.g., as follows: by at least one but less than 10, 20, 30, or 40 nucleotides; at least one but less than 1%, 5%, 10% or 20% of the nucleotides in the subject nucleic acid. If necessary for this analysis the sequences should be aligned for maximum homology. “Looped” out sequences from deletions or insertions, or mismatches, are considered differences. The differences are, preferably, differences or changes at nucleotides encoding a non-essential residue(s) or a conservative substitution(s).

The terms “host cell” and “recombinant host cell” are used interchangeably herein. Such terms refer not only to the particular subject cell, but also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic, e.g., bacterial cells such as E. coli, or eukaryotic, e.g., insect cells, yeast, or preferably mammalian cells (e.g., cultured cell or a cell line). Other suitable host cells are known to those skilled in the art.

Useful mammalian host cells for expressing the anergy-associated nucleic acids of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), lymphocytic cell lines, e.g., NS0 myeloma cells and SP2 cells, COS cells, HEK cells, and a cell from a transgenic animal, e.g., e.g., mammary epithelial cell.

Included within the present invention are vectors, e.g., a recombinant expression vector. The recombinant expression vectors of the invention can be designed for expression of the anergy-associated nucleic acids, in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of nucleic acids to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “regulatory sequence” is intended to include promoters, enhancers and other expression control elements (e.g., polyadenylation signals) that control the transcription or translation of genes. Such regulatory sequences are described, for example, in Goeddel; Gene Expression Technology: Methods in Enzymology 185, Academic Press, San Diego, Calif. (1990). The design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the nucleic acids and regulatory sequences, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

Standard recombinant DNA methodologies are used to obtain anergy associated nucleic acids, incorporate these nucleic acids into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989).

The invention is illustrated in part by the following examples, which are not to be taken as limiting the invention in any way.

### EXAMPLES

### Example 1

### Assay for Ubiquitin Ligase Activity of HECT-Type E3 Ligases

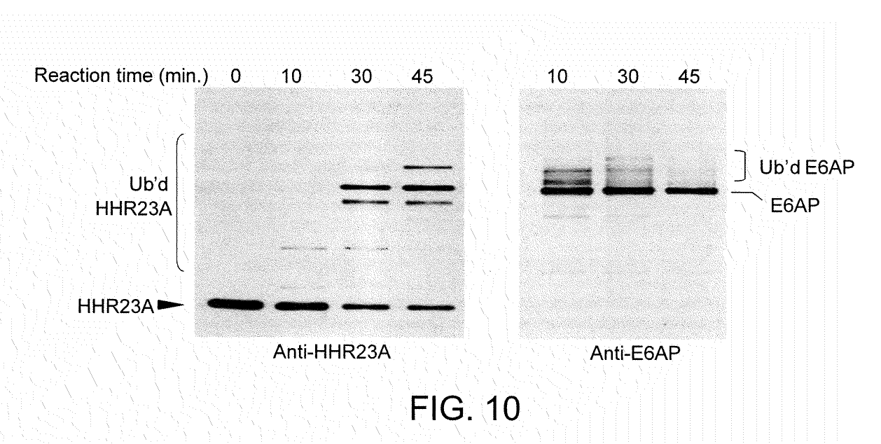

HECT-type E3 ligases can auto-ubiquitinate themselves by transferring ubiquitin from the catalytic cysteine (thio-ester bond) to adjacent ε-amino groups of appropriately positioned lysine residues in the HECT domain or other nearby domains. FIG. 10 documents auto-ubiquitination of full-length E6AP protein. To generate the data in FIG. 10, reactions containing bacterially-expressed HHR23A substrate, purified E6AP, E1, E2 (UbCH7), ubiquitin and ATP were resolved by SDS-PAGE and immunoblotted with antibodies against HHR23A (lanes 1-4) and E6AP (lanes 5-7). As can be seen in FIG. 10, there is marked ubiquitination at 10 min, a time when trans-ubiquitination of the substrate HHR23A is just barely detectable. In contrast to the time-dependent increase in the amount of ubiquitin-conjugated HHR23A substrate, there was apparently no increase in the amount of self-Ub-E6AP conjugates with time (lanes 5-7). This however was an artifact caused by inefficient transfer of high molecular weight, poly-ubiquitinated E6AP to the blot, since similar experiments with in vitro-translated, ³⁵S-labelled E6AP showed increasing levels of poly-ubiquitinated forms with increasing times of incubation.

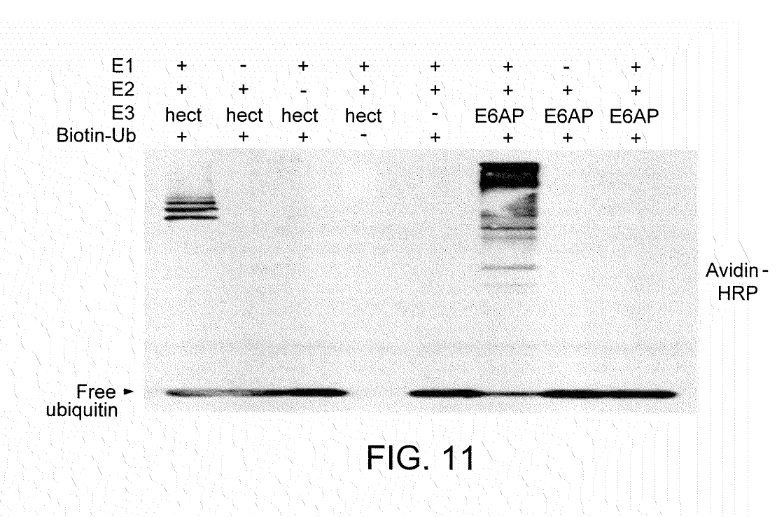

FIG. 11 shows that the HECT domain of E6AP is sufficient for self-ubiquitination. To generate the data in FIG. 11, reactions containing bacterially-expressed E6AP HECT domain or insect cell-expressed full-length E6AP, E1, E2, and biotin-Ub were resolved by SDS-PAGE and probed with avidin-HRP to detect Ub conjugates. As can be seen in FIG. 11, all components, i.e., E1, E2 (UbCH7), the HECT domain and ubiquitin, are required for self-ubiquitination. In other experiments, ATP was also shown to be essential.

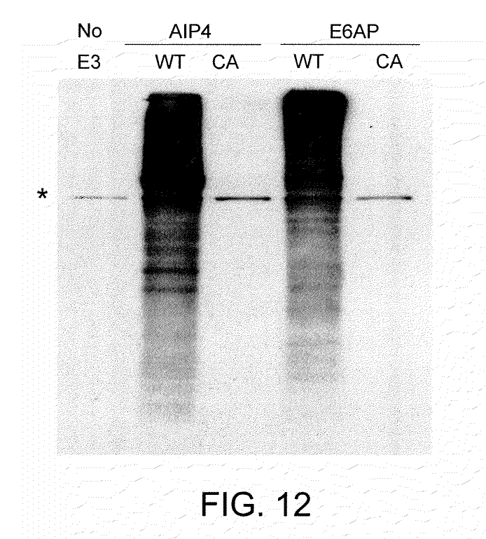

FIG. 12 shows auto-ubiquitination of AIP4, the human homologue of Itch, with E6AP as positive control. To generate the data in FIG. 12, reactions containing insect cell-expressed full-length wild type (WT) or catalytically-inactive (C>A) mutant AIP4 or E6AP, purified E1, E2 (UbCH7), and biotin-Ub were resolved by SDS-PAGE and probed with avidin-HRP to detect ubiquitin conjugates. The AIP4 and E6AP-dependent smears likely represent ubiquitin conjugated to full-length E3 enzymes or E3 proteolytic fragments, as well as some free ubiquitin chains. Asterisk, non-specific band. Thus, the AIP4 reagent was validated, and AIP4 and Itch are very highly homologous at the sequence level.

### Example 2

### In Vitro Assays for Screening for Inhibitors of Ubiquitin Ligases

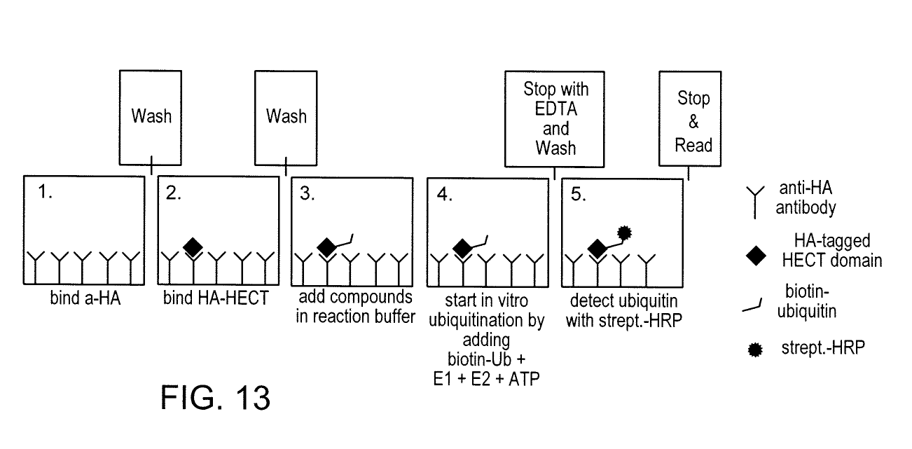

The design of one assay is based on monitoring auto-ubiquitination of Itch or its human homologue AIP4 (see FIG. 13). Briefly, the HECT domains of Itch and AIP4 (e.g. Itch amino acids 439-850), fused to the HA epitope tag at their N-termini, are expressed as GST fusion proteins in bacteria, cleaved with Precission protease to remove the GST, then immobilized in 96-well or 384-well plates that are coated with the 12CA5 antibody to the HA tag (steps 1 & 2 of FIG. 13). After washing, a robot can be used to dispense library compound into the wells (step 3 of FIG. 13). After a brief incubation period (10-biotin-Ub and ATP are added to each well (step 4 of FIG. 13). After an incubation period determined separately to give optimal signal-to-noise ratio for biotin-Ub transfer, the reaction is stopped with 10 mM EDTA, the plates are washed, allowed to bind streptavidin-HRP (horseradish peroxidase) (step 5 of FIG. 13), washed again, and developed with substrate for colorimetric detection on an ELISA plate reader. Compounds that show inhibition in the assay are rescreened at varying doses in high-throughput format to provide an estimate of inhibitory potency (Ki), and are also screened in a standard assay involving analysis by SDS-PAGE (discussed in Example 1, see FIGS. 10 to 12). A FRET-based assay suitable for monitoring ubiquitin transfer in a high-throughput format has been described in the literature (see, e.g., Boisclair et al., (2000) J Biomol Screen 5:319) and could be adapted for use in the presently described system.

In another assay, the ability of HECT-type and adaptor-type E3 ubiquitin ligases to ubiquitinate cellular substrates can be tested in vitro. The design of the library screen is exactly as depicted in FIG. 13 except that the reaction step contains not only E1, E2, biotin-Ub and ATP but also the substrate and any other adapters or cofactors that might be needed for efficient transubiquitination. Compounds that show inhibition are rescreened at varying doses, and the compounds with greatest inhibitory potency are subjected to secondary screening.

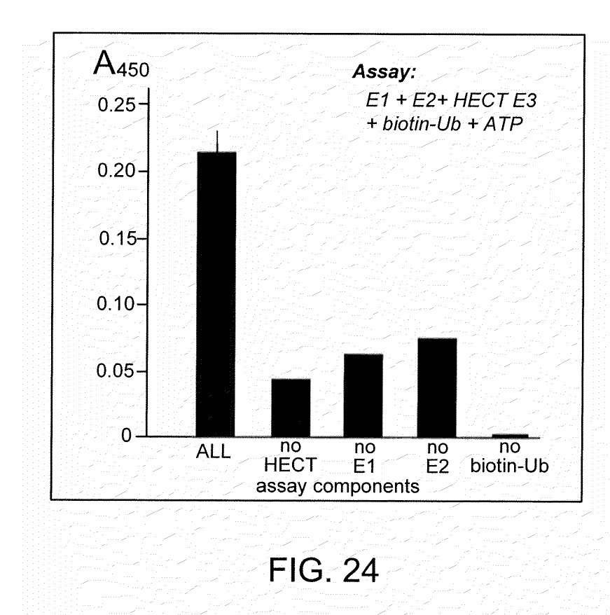

FIG. 24 provides results obtained using an assay as described in the present specification. In this assay, 96-well plates were coated with anti-HA, washed, quenched with PBS-BSA, and used to immobilize the HA-tagged HECT domain of E6AP. The reaction was initiated by addition of E1, E2, biotin-Ub and ATP, following which the wells are washed thoroughly, allowed to bind streptavidin-HRP, and developed with substrate in an ELISA format. The reaction with all components (E1, E2, HECT, biotin-Ub and ATP) showed strong colour development (see FIG. 24, left bar). The reaction lacking biotin-Ub is blank as expected (right bar). The other three reactions (lacking E1, E2 or HECT) show background absorbance, which could be due to nonspecific sticking of biotin-Ub to the wells, covalent transfer of the biotin-Ub from one of the remaining Ub ligases (E1, E2 or HECT) to the anti-HA antibody coating the wells, or both.

### Example 3

### Calcineurin Imposes T Cell Unresponsiveness Through Targeted Proteolysis of Signaling Proteins

### Mice

BALB/cJ, DO11.10 and 2B4 TCR-transgenic mice were obtained from Jackson laboratories, held and bred under pathogen-free conditions in a barrier facility.

### Induction of Oral Tolerance In Vivo

Female DO11.10 TCR-transgenic mice (6 to 8 weeks) received ovalbumin either in the drinking water as described earlier or were given gastric injections of 28 mg OVA in 0.7 ml PBS on two consecutive days (days 1 and 2), and sacrificed on day 4 for T cell isolation from spleen and lymph nodes. Age- and sex-matched littermate controls received identical injections of PBS alone.

### Cell Culture, Cell Stimulation and Anergy Induction Ex Vivo

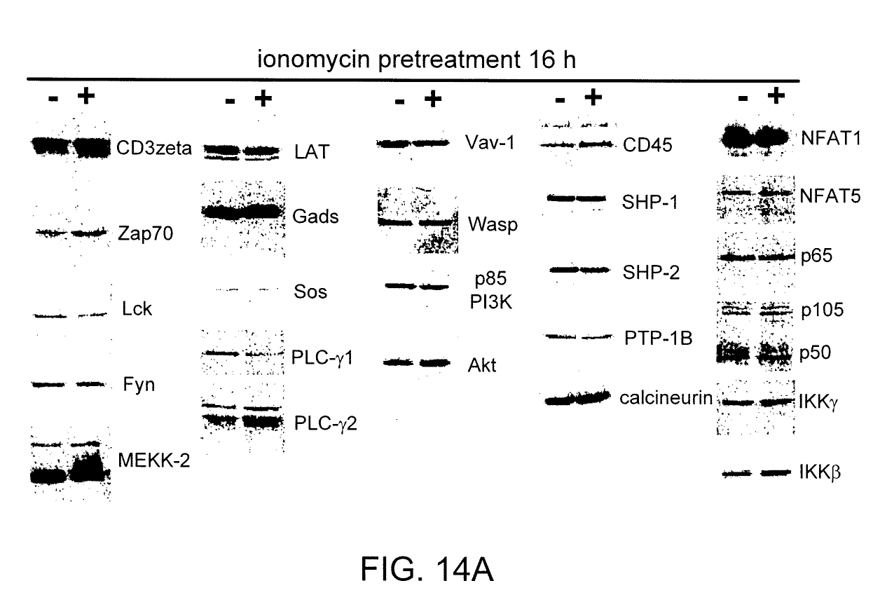

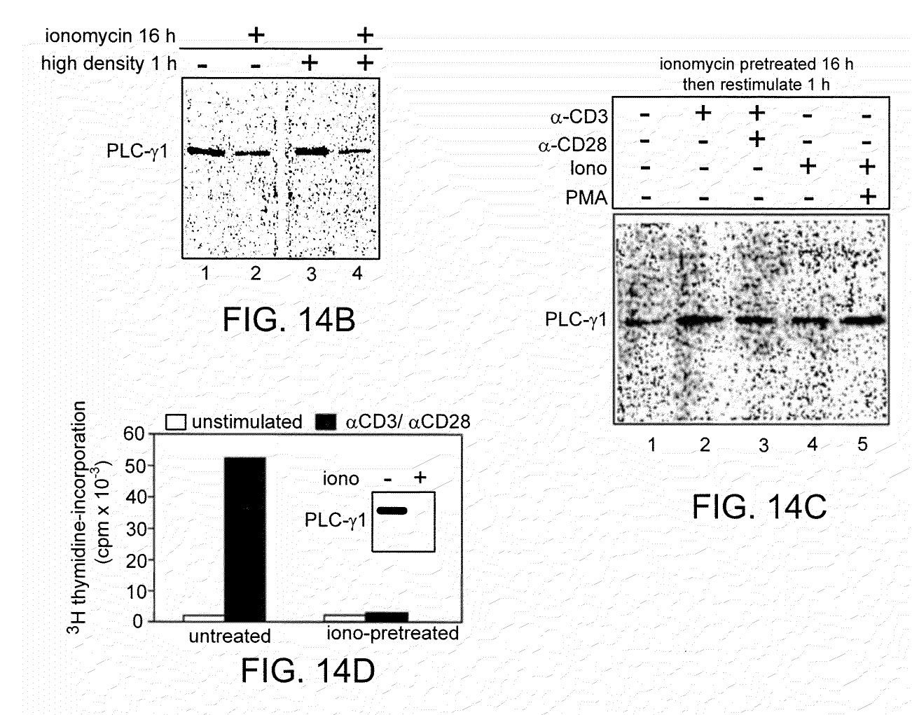

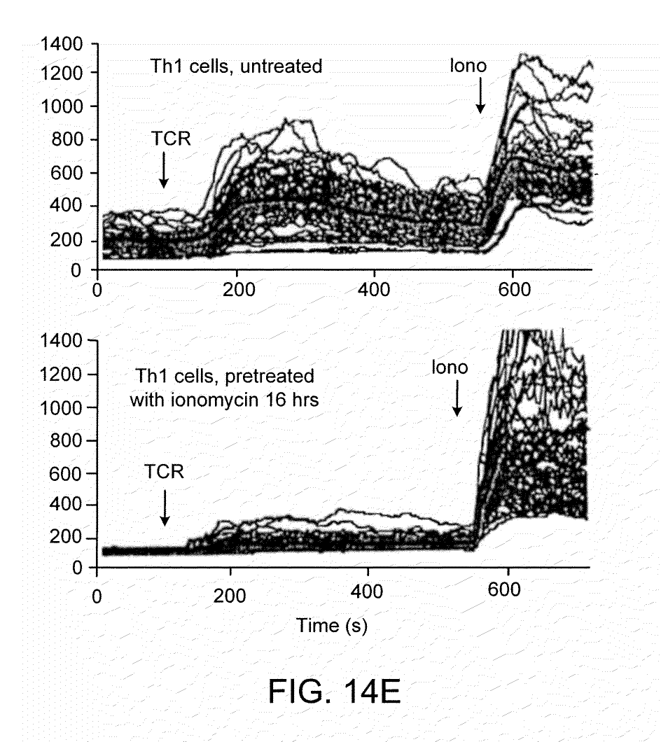

The murine D5 (Ar-5) Th1 cell clone was grown as previously described (F. Macian et al., Cell 109, 719-31. (2002). CD4+ cells were isolated from spleen and lymph nodes of DO11.10 or 2B4 TCR-transgenic mice using positive selection with anti-CD4 magnetic beads (Dynal), and differentiated into Th1 cells for 2 weeks using standard protocols (id.). Anergy was induced by treating primary Th1 cells or the D5 Th1 clone (106 cells/ml) with 1 μM ionomycin for 16 hours, Cyclosporin A was included in some experiments at a concentration of 2 μM. The cells were then washed to remove the ionomycin and incubated at higher cell density (˜3×10⁶ cells/ml) for 1-2 hours at 37 C. In the experiment of FIG. 14, a high-density incubation step was included. The extent of anergy induction was evaluated by intracellular cytokine staining or in standard proliferation assays (id.). Restimulation of D5 cells was done with 1 μg/ml anti-CD3 with or without 2.5 μg/ml anti-CD28 or with 20 nM PMA or 1 μM Ionomycin or both. HEK 293 cells were grown and transfected with Ca2+ phosphate using standard protocols.

### Antibodies and Expression Plasmids

Antibodies against Zap70, Lck, PKCθ, Itch and calcineurin were obtained from BD Transduction Labs. Antibodies to Fyn, RasGAP, SOS, Vav-1 and Nedd4 were purchased from Upstate Biotechnologies. Santa Cruz antibodies were used to detect CD3δ, Mekk-2, RasGRP, ubiquitin, PLC-γ2, Cbl-b, NFκB p65, NFκB p50, IKK_{γ}, Myc- and HA-tagged proteins. Antibody to the AU.1 epitope tag was purchased from Covance, anti-Akt from Cell signaling, anti-Tsg101 from Genetex and anti-IKKβ from Biosource. Antibodies against NFAT1 and NFAT5 were produced in the lab and antibodies against Gads, LAT, p85 PI3K, SHP-1, SHP-2, and PTP-1B were obtained. Endogenous PLC-γ1 was detected with a polyclonal antiserum that was raised against the epitope APRRTRVNGDNR (SEQ ID NO:31) representing the very C-terminal amino acids of the protein. Importantly the epitope does not contain any tyrosine residues and only one threonine residue, which is not part of any predictable phosphorylation motif as judged by the Scansite computer program. Furthermore a commercial antibody source, comprising a pool of 4 different monoclonal antibodies (Upstate Biotechnologies), also allowed visualization of the differences in PLC-γ1 protein levels in untreated and anergic T cells, when the antibody was used at a 5 fold higher dilution than recommended.

### Expression Plasmids

Nedd4 (KIAA0093) and Itch cDNAs were inserted via SalI/NotI into pRK5 vectors containing an amino-terminal sequence coding for the myc epitope.

### Cell Extracts, Immunoprecipitations and Immunoblots

D5 cells were extracted at 106 cells/10 μl in RIPA buffer (20 mM Tris pH 7.5, 250 mM NaCl, 1 mM DTT, 10 mM MgCl2, 1% Nonidet P-40, 0.1% SDS, 0.5% sodium deoxycholate) supplemented with protease and phosphatase inhibitors (1 mM PMSF, 25 μg/ml aprotinin, 25 μg/ml leupeptin, 10 mM NaF, 8 mM βglycerophosphate, 0.1 mM sodium ortho vanadate). For assessing protein levels in cell extracts, 5-30 μl of RIPA extracts were separated on 9-12% SDS-polyacrylamide gels, and proteins were electrotransferred onto nitrocellulose membranes. For immunoprecipitations, 500-1000 μl of RIPA cell extracts were used. For coimmunoprecipitations from lysates of transfected HEK 293 cells, cells from one 10 cm dish were lysed in 50 mM Hepes pH 7.5, 100 mM NaCl, 1 mM EDTA, 0.5% NP-40 and 10% glycerol including phosphatase and protease inhibitors. Lysates were precleared with either protein A- or protein G-Sepharose, immunoprecipitations were performed for 4 hrs and the resulting precipitates were washed 3-4 times with the buffer used for cell extraction. Immunoblots were performed with antibody solutions in 5% milk and TBS (10 mM TrisCl (pH 8.0), 150 mM NaCl) and washes were done in TBS containing 0.05% Tween-20.

### Metabolic Labeling and Pulse Chase Experiments

CD4 cells were isolated via dynal beads selection, cells were starved for 1 hr in cysteine/methionine free media and incubated for 2 hrs with 100 μCi/ml 35S-cysteine and -methionine. Cells were washed, resuspended in complete media and stimulated with 2 μg/ml anti-CD3 on crosslinking antibody coated plates. Cells were extracted in RIPA buffer and immunoprecipitations performed as described above. Immunoprecipitates were resolved on SDS-PAGE, that were treated with En3hance solution (NEN), dried and used for autoradiographs. Densitometric analysis was performed using IQ-Mac vs 1.2 software.

### Cell Fractionation

Cell fractionation was performed essentially as described (Khoshnan et al. J. Immunol. 165, 6933-40 (2000)) using 3×10⁷ D5 cells. Cells were swollen for 15 min in hypotonic buffer E (10 mM Tris pH 7.4, 10 mM KCl, 1.5 mM MgCl2, 1 mM DTT supplemented with protease and phosphatase inhibitors) and lysed by douncing. Lysates were centrifuged at 100,000 g for 30 min yielding a supernatant (“cytosol”) and a pellet that was resuspended in buffer E containing 1% NP-40 and recentrifuged at 100,000 g for 30 min to separate the detergent-soluble fraction in the supernatant from the detergent-insoluble fraction (pellet). The pellet was resuspended by sonication in RIPA buffer and cleared by centrifugation before analysis of all fractions by immunoblotting.

### [Ca]ᵢ Imaging and Immunocytochemistry

Intracellular calcium measurements were performed on primary Th1 cells from 2B4 mice or on CD4+ T cells isolated by negative selection using separation columns (RnD systems) from spleen and lymph nodes of DO11.10 TCR transgenic mice, that were either left untreated or rendered tolerant by gastric injections of high doses of ovalbumin. Cells were loaded with 1 μM fura-2 AM (Molecular Probes) for 30 min at room temperature, washed and resuspended in loading medium (RPMI+10% FCS), incubated with 2.5 μg/ml biotinylated anti-CD3 (2C11, Pharmingen) for 15 min at room temperature and attached to poly-L-lysine coated coverslips mounted in a RC-20 closed bath chamber (Warner Instrument Corp., Hamden, Conn.). The fura-2-loaded cells were perfused in Ringer solution containing 2 mM calcium (155 mM NaCl, 4.5 mM KCl, 10 mM D-glucose, 5 mM Hepes (pH 7.4), 1 mM MgCl₂, 2 mM CaCl₂) and stimulated by crosslinking the surface-bound biotinylated anti-CD3 with 2.5 μg/ml streptavidin (Pierce), following which healthy cells were identified by their responsiveness to 1 μM ionomycin (Calbiochem). Single cell video imaging was performed on an Zeiss Axiovert 5200 epifluorescence microscope using OpenLab imaging software (Improvision). Fura-2 emission was detected at 510 nm following excitation at 340 and 380 nm, respectively. 340/380 ratio images were acquired every 5 seconds after background subtraction. Calibration values (Rmin, Rmax, Sf) were derived from cuvette measurements using a calcium calibration buffer kit (Molecular Probes) and as previously described (Grynkiewicz et al. J Biol Chem 260, 3440-50 (1985)).

### Real-Time PCR Analysis

Total RNA was prepared from untreated or ionomycin-pretreated D5 cells using Ultraspec reagent (Biotecx). cDNAs were synthesized from 2 μg of total RNA as template, using a cDNA synthesis kit (Invitrogen). Quantitative real time-PCR was performed in an I-Cycler (BioRad) using a SYBR Green PCR kit (Applied Biosystems). The sequences of the primer pairs are as follows:

| | |
|---|---|
| L32 sense | |
| 5′-CGTCTCAGGCCTTCAGTGAG-3′; | (SEQ ID NO: 30) |
| L32 anti-sense | |
| 5′-CAAGAGGGAGAGCAAGCCTA-3′; | (SEQ ID NO: 21) |
| PLC-γ1 sense | |
| 5′-AAGCCTTTGACCCCTTTGAT-3′; | (SEQ ID NO: 22) |
| PLC-γ1 anti-sense | |
| 5′-GGTTCAGTCCGTTGTCCACT-3′; | (SEQ ID NO: 23) |
| Itch sense | |
| 5′-GTGTGGAGTCACCAGACCCT-3′; | (SEQ ID NO: 24) |
| Itch anti-sense | |
| 5′-GCTTCTACTTGCAGCCCATC-3′; | (SEQ ID NO: 25) |
| Cbl-b sense | |
| 5′-CTTAAATGGGAGGCACAGTAGAAT-3′; | (SEQ ID NO: 26) |
| Cbl-b anti-sense | |
| 5′-CAGTACACTTTATGCTTGGGAGAA-3′; | (SEQ ID NO: 27) |
| Grail sense | |
| 5′GTAACCCGCACACCAATTTC-3′; | (SEQ ID NO: 28) |
| and | |
| Grail anti-sense- | |
| 5′GTGAGACATGGGGATGACCT3′. | (SEQ ID NO: 29) |

Thermal cycling conditions were 95° C. for 5 min, then 40 cycles of 95° C., 65° C., and 72° C. for 30 sec each, terminating with a single cycle at 72° C. for 5 min. Signals were captured during the polymerization step (72° C.). A threshold was set in the linear part of the amplification curve, and the number of cycles needed to reach it was calculated for each gene. Melting curve analysis and agarose gel electrophoresis were performed to test the purity of the amplified bands. Normalization was performed by using L32 levels as an internal control for each sample. The ratio of mRNA levels in ionomycin-treated or ionomycin/CsA treated to untreated samples were determined.

### Formation of Immunological Synapses in Lipid Bilayers

Planar bilayers were prepared essentially as described in (Grakoui et al., Science 285, 221-7 (1999)), except that the MCC88-103 peptide was loaded on the GPI-IEk for 24 hours. Bilayers were prepared using Oregon green labeled GPI-IEk and Cy5 labeled GPI-ICAM-1 in parallel plate flow cells (Bioptechs). Control and ionomycin treated cells were injected into the flow cell at a density of 10⁶ cells/ml. Areas of bilayers where cells were forming synapses were imaged using FITC and Cy5 optics on an Olympus IX-70 inverted microscope equipped with a amamtsu ORCA-ER digital camera and a Xenon-arc lamp as a light source for fluorescence microscopy. The filter wheels, shutters and the camera were controlled using the IPLAB software on a Macintosh platform. Bright field, interference reflection (IRM) and fluorescence images were collected and processed using the Metamorph software. The background from the fluorescence images was subtracted using the produce background correction image function which is based on median filtering to subtract background that is nonuniform. Percentage of cells adhering were analyzed by comparing bright field and IRM images.

Experiments using phospholipase inhibitors were performed using AND T cell blasts (day 8). Cells were allowed to form immunological synapses on bilayers containing 80 molecules/μm² of Oregon green E^{k}-MCC 88-103 and 200 molecules/μm² of Cy5 ICAM-1 in the presence of 0.01% DMSO (the carrier concentration for 1 μM U73122 and U73343). After 60 minutes, fields containing stable immunological synapses with central MHC clusters (green) and complete ICAM-1 rings (red) were imaged and the locations recorded using an automated stage and IPLab software. The stable synapses were then treated sequentially with 1 μM U73343 and 1 μM U73122 (weak and strong PLC-γinhibitors, respectively). After each drug treatment the same fields were imaged within 10 minutes so that the effects of the drugs on many individual synapses could be determined. The quantitative data reflect the percentage of intact LFA-1/ICAM-1 rings after carrier or drug treatment on 103 contact areas. In separate experiments it was shown that the effects of U73343 and U73122 were stable for up to 1 hr and that U73122-dependent destruction of the LFA-1 adhesion ring was not dependent upon prior treatment with U73343. These effects were observed in 3 independent experiments with U73122 concentrations from 0.1-1 μM.

### Receptor Stimulation as an Inhibitor of T-Cell Signaling

Besides activating signaling pathways that have a positive effect, receptor stimulation induces negative feedback pathways that attenuate or terminate positive signaling, thus ensuring a balanced response to extracellular signals and protecting cells from the deleterious effects of chronic activation. In one well-documented mechanism, activated signal transducers are selectively targeted for degradation, terminating ongoing signals and also interfering with subsequent stimulation. Cytoplasmic signaling proteins and nuclear transcription factors tend to be polyubiquitinated and targeted for proteasomal degradation (Harris et al., Proc Natl Acad Sci USA 96, 13738-43. (1999), Lo et al. Nat Cell Biol 1, 472-8. (1999)), whereas ligand-activated surface receptors, including receptor tyrosine kinases, G protein-coupled receptors, and the T cell receptor (TCR) are more often degraded by tagging of receptor or adaptor proteins with mono-ubiquitin, followed by endocytosis, sorting into multivesicular bodies at the endosomal membrane and trafficking to the lysosome (Sorkin et al., Nat Rev Mol Cell Biol 3, 600-14 (2002); Valitutti et al., J Exp Med 185, 1859-64 (1997)). Preactivation of negative signaling can shift the temporal balance of positive activation, leading to blunted responses or even complete loss of signal transduction in response to a subsequent stimulus. Ca2+ signaling in the immune system, which has both positive and negative effects, provides an example. In T cells, sustained elevation of Ca2+ and activation of calcineurin are essential for persistent nuclear translocation of the transcription factor NFAT, which in turn induces a very large number of cytokine, chemokine and other genes important for the productive immune response (Macian et al., Oncogene 20, 2476-89 (2001), Feske et al., Nat Immunol 2, 316-24 (2001)). The same transcription factor, when preactivated in the absence of its transcriptional partner AP-1 (Fos-Jun), induces a different set of genes encoding known or presumed negative regulators of T cell signaling, thus mediating an opposing program of T cell anergy or tolerance (Macian et al., Cell 109, 719-31 (2002)).

### Alterations in Signalling Proteins in Anergized Immune Cells

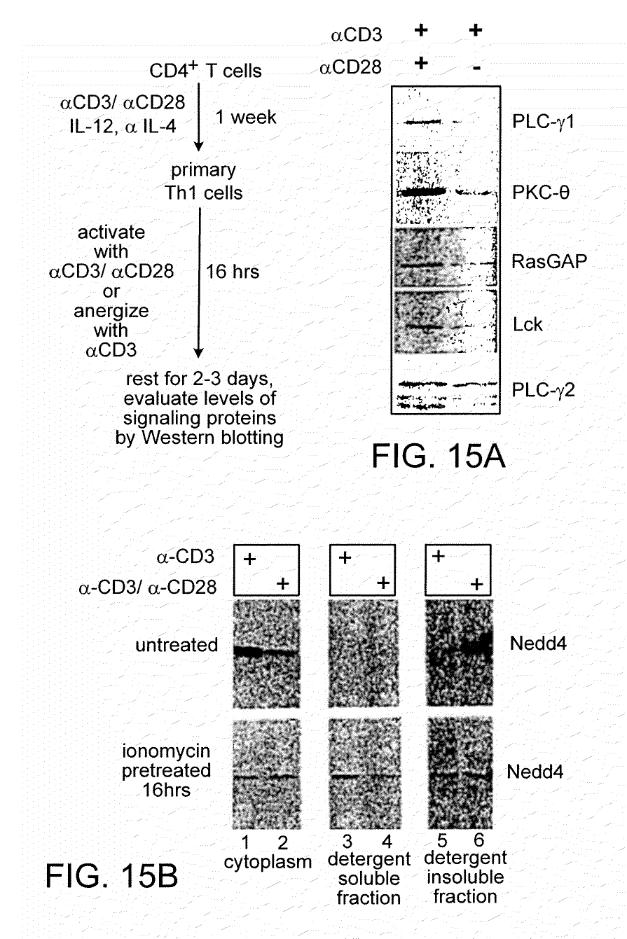

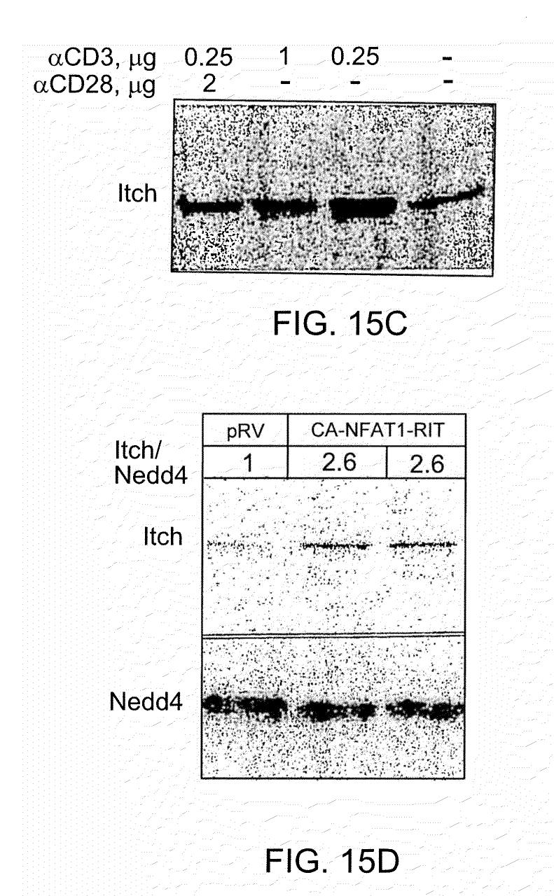

The levels of a large number of signaling proteins in cells anergized by sustained exposure to ionomycin or immobilized anti-CD3 was assessed (FIGS. 14A and 15A). A surprisingly limited number of changes was observed, among them a reproducible decrease in intensity of the PLC-γ1 band (FIGS. 14A and 15A). The decrease required not only ionomycin pretreatment, but also restimulation or formation of cell-cell contacts (FIGS. 14B, C, and D). Decreases of PLC-γ1 and other signaling proteins were also observed in primary T cells anergized with anti-CD3 (FIG. 15A).

The levels of most signaling proteins showed little or no alteration after ionomycin-pretreatment of the D5 Th1 clone: the most striking changes were an apparent protein modification occurring on MEKK-2 (FIG. 14A; column 1) and a clear decrease in protein levels of PLC-γ1 (FIG. 14A; column 2). Notably, there was no change in PLC-γ2, protein levels in the same cell extracts (FIG. 14A; column 2). A slight reduction of signal for the Lck protein was also observed in some experiments (FIG. 14A; column 1); this effect appeared more prominent in primary T cells than in D5 T cells (FIG. 15A). Focus was initially on the decrease in PLC-γ1 protein levels in anergic D5 T cells. The extent of decrease was variable in cells assayed directly after the period of ionomycin pretreatment, even though the cells could be shown to be markedly anergic in a parallel proliferation assay. Cells that were insufficiently anergized never showed a strong decrease. Cells in the ionomycin-treated cultures formed large, macroscopically visible aggregates, which developed slowly during the period of ionomycin treatment and were particularly obvious if the cells were centrifuged to wash away ionomycin and then incubated at high cell density. The aggregates were not observed with parallel cultures of untreated T cells, nor were they observed with cells treated with ionomycin in the presence of CsA, indicating that aggregate formation required calcineurin activity. It was noticed that formation of large cell aggregates correlated with the highest levels of anergy induction (i.e. the lowest responses in a subsequent stimulation step) and with the greatest decreases in PLC-γ1 levels, especially in cells incubated briefly at 37° C. before lysis.

These findings led us to suspicion that the major change in PLC-γ1 levels occurred not during ionomycin pretreatment, but rather during the subsequent period of cell incubation in the proliferation assay (see FIGS. 14B, 14C, and 14D). Decrease in PLC-γ1 levels was not due to cell death occurring under these conditions. It was also not due to downregulation of PLC-γ1 gene transcription, since PLC-γ1 mRNA levels were unaffected in anergic D5 T cells (see FIG. 18B). PLC-γ1 did not relocalize to a different intracellular compartment that was susceptible to detergent extraction: when the DNA-containing pellets remaining after cell lysis with RIPA buffer were re-extracted with SDS, no residual PLC-γ1 was detected in either untreated or anergic T cells (data not shown). Finally, the decrease did not reflect posttranslational modification and consequent loss of reactivity with the immunoblotting antibody, as previously postulated, since it was observed with two different antibodies to PLC-γ1 and PKCθ. It appears that anergic T cells degrade PLC-γ1 in two separable stages. A period of sustained Ca2+/calcineurin signaling is required to initiate the degradation program, but degradation is actually implemented during a subsequent step of TCR stimulation or the surrogate stimulus provided by homotypic cell adhesion. LFA-1/ICAM-1 interactions are implicated in both cases, but the independent role of TCR/MHC versus LFA-1/ICAM-1 interactions in promoting degradation of PLC-γ1, PKCθ or other signaling proteins, has not been examined.

### Anergy is Mediated Through Ca2+/Calcineurin-Dependent Degradation Program

In experiments performed under optimized conditions, there was a strong correlation between loss of PLC-γ1 and extent of anergy induction in a parallel proliferation assay (FIG. 14D). As expected from the central role of PLC-γ1 in Ca2+ mobilization and T cell activation, anergic T cells showed decreased Ca2+ fluxes in response to TCR stimulation (FIG. 14E). Thus, T cell anergy was strongly correlated with PLC-γ1 degradation; the degradation program was initiated by sustained Ca2+/calcineurin signaling, but degradation was actually implemented after formation of cell-cell contacts (T-T or T-APC).

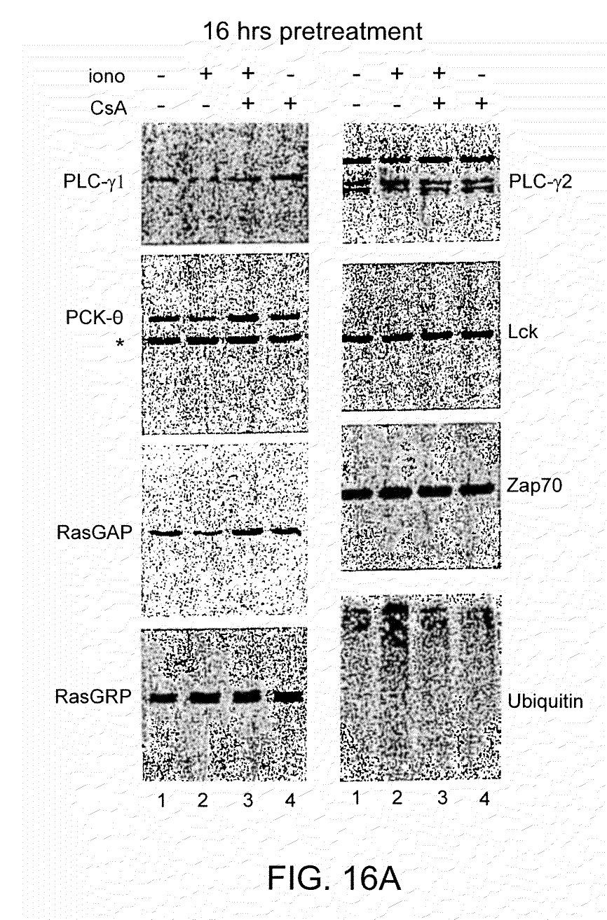

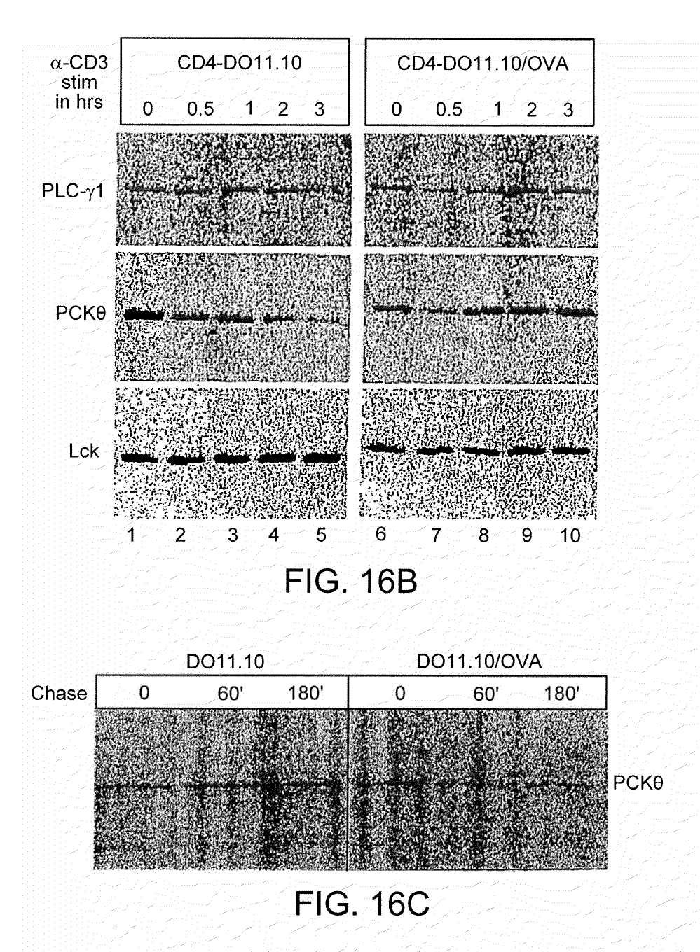

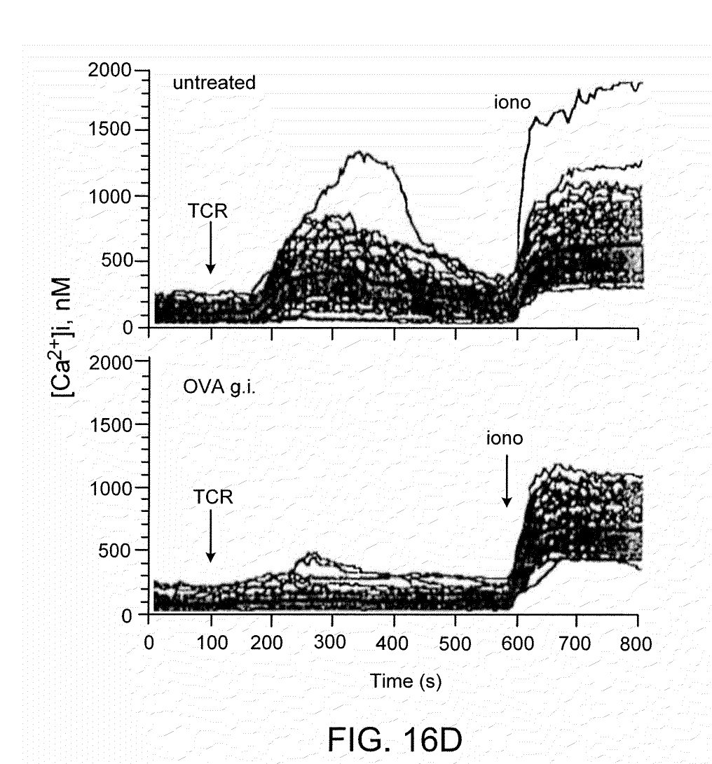

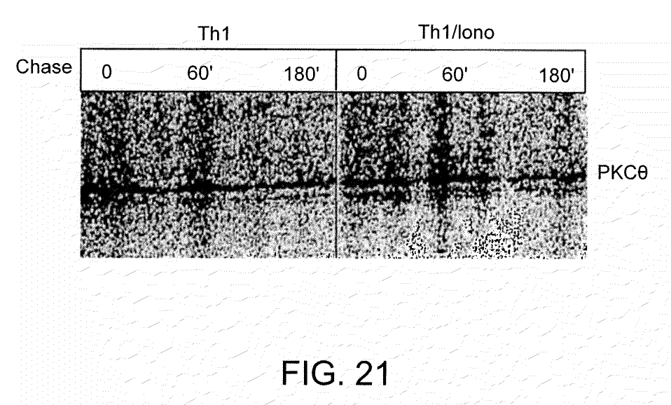

Since lymphocyte anergy and tolerance are imposed by Ca2+/calcineurin signaling, the role of calcineurin in PLC-γ1 degradation was evaluated (FIG. 16A). D5 T cells subjected to ionomycin pretreatment followed by cell-cell contact showed a pronounced decrease of PLC-γ1, PKCθ and RasGAP protein levels, but no change in the levels of several other signaling proteins, RasGRP, Lck, ZAP70, and PLC-γ2 (FIG. 16A). Degradation was completely blocked by including the calcineurin inhibitor cyclosporin A (CsA) during the ionomycin treatment step (FIG. 16A). Pulse-chase experiments showed that PKCθ from ionomycin-treated T cells turned over significantly more rapidly than PKCθ from mock-treated T cells (FIG. 21), demonstrating that decreased intensity in Western blots was due to accelerated degradation of the signaling proteins and not decreased gene transcription, epitope masking or altered compartmentalization. Ionomycin pretreatment also induced a ˜2-fold increase in total protein ubiquitination which was blocked by cyclosporin A, suggesting that Ca²⁺/calcineurin signaling activated ubiquitin dependent proteolytic pathways (FIG. 16A).

Whether loss of PLC-γ1 could also be observed in T cells anergized in vivo was also investigated (FIG. 16B). A model of oral tolerance to ovalbumin (OVA) was used, in which high antigen doses rapidly induce T cell anergy in DO11.10 TCR-transgenic mice; high dose antigen administered for short times results in T cell anergy whereas low dose antigen induces suppression via regulatory T cells. No difference could be detected in the levels of PLC-γ1 or PKCθ in unmanipulated CD4 T cells isolated from untreated and OVA-tolerized mice (FIG. 16B, lanes 1 and 6); in contrast, anti-CD3 stimulation induced an early (0.5-1 h) and selective decrease of PLC-γ1 and PKCθ levels in T cells from OVA-tolerized mice (FIG. 16B; lanes 7, 8) but not in T cells from untreated mice (FIG. 16B; lanes 2, 3). At later times (2-3 h), protein levels were restored in T cells from tolerant mice (FIG. 16B; lanes 9, 10), but declined in T cells from untreated mice, suggesting that the degradation observed in anergic cells was primarily associated with the initial phase of TCR stimulation and was counteracted by protein resynthesis at later times, and moreover that degradation could be an early manifestation of a downregulatory program normally turned on late in T cell activation. Pulse-chase experiments confirmed that PKCθ from in vivo-tolerized T cells had a significantly shorter half-life than observed in untreated T cells (FIG. 16C). Consistent with PLC-γ1 degradation, both ex vivo-anergized and in vivo-tolerized T cells displayed a marked impairment of Ca²⁺ mobilization in response to TCR crosslinking (FIGS. 14E and 16D).

To determine the time course of protein degradation, pulse-chase experiments were performed (FIG. 16C). PKCθ from in vivo-tolerized T cells indeed displayed a significantly shorter half-life, relative to PKCθ from untreated T cells (compare FIG. 16C lanes 4-6 with lanes 1-3). After 60 minutes of anti-CD3 stimulation, the levels of radiolabeled PKCθ showed a striking decline, to 58% of initial levels, in T cells from tolerized mice (FIG. 16C; lanes 4-6); in contrast, the level increased slightly, to 110% of initial levels, in T cells from untreated mice (FIG. 16C; lanes 1-3), presumably due to incorporation of residual labeled amino acids as a result of transcription/translation stimulated by anti-CD3. At 2-3 h, PLC-γ1 and PKCθ levels declined slightly even in T cells from untreated mice as judged by western blotting (FIG. 16B, lanes 4, 5), suggesting that the degradation observed in anergic T cells might be an early manifestation of a downregulatory program that is normally turned on late in T cell activation.

These results (FIGS. 16 and 14) again emphasize that although tolerant cells are primed to initiate a limited program of protein degradation, degradation only occurs when the primed cells are subsequently stimulated. The effect on signaling is rapid and pronounced, however: like T cells anergized in vitro (FIG. 14E), in vivo-tolerized T cells displayed a marked impairment of Ca2+ mobilization in response to TCR crosslinking (FIG. 16D). The data indicate that the active, membrane-proximal pool of signaling proteins is rapidly and preferentially degraded in anergic T cells, while the inactive fraction is spared.

### Ubiquitin Ligases Mediate the Degradation of Signaling Proteins in Anergized Immune Cells

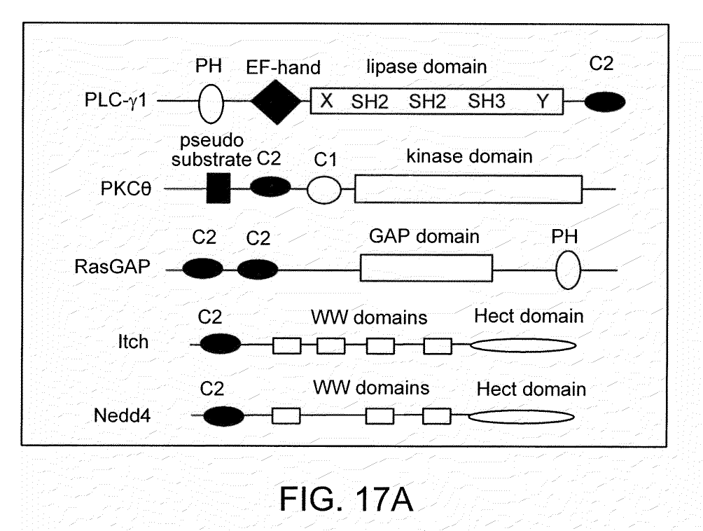

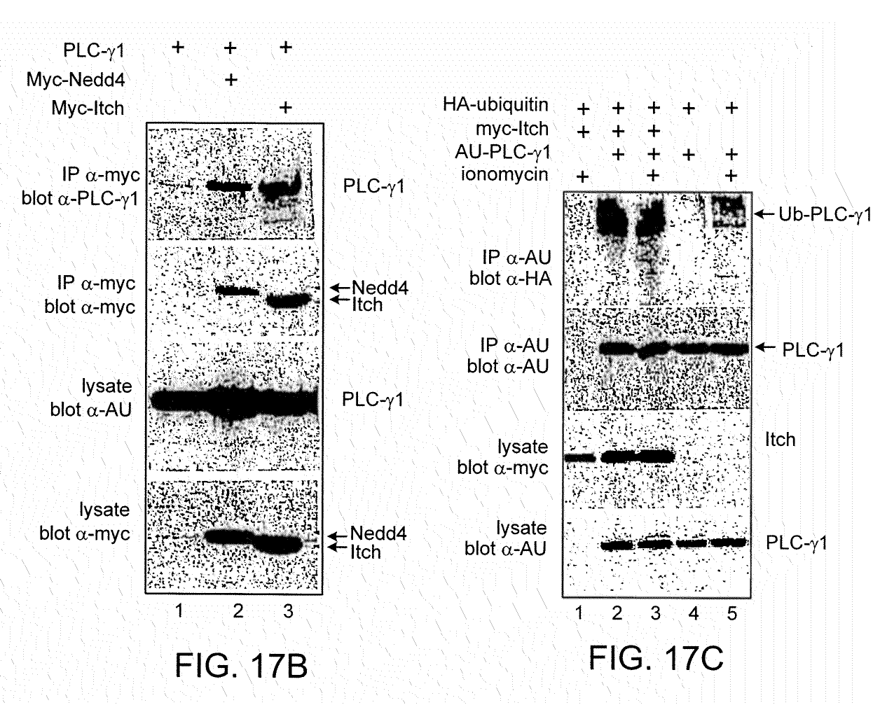

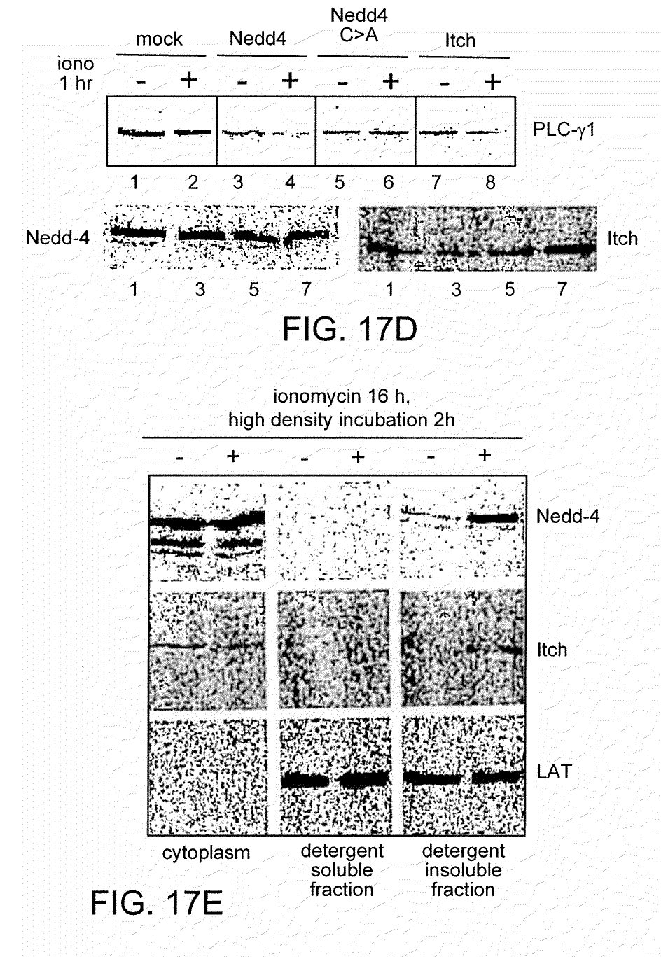

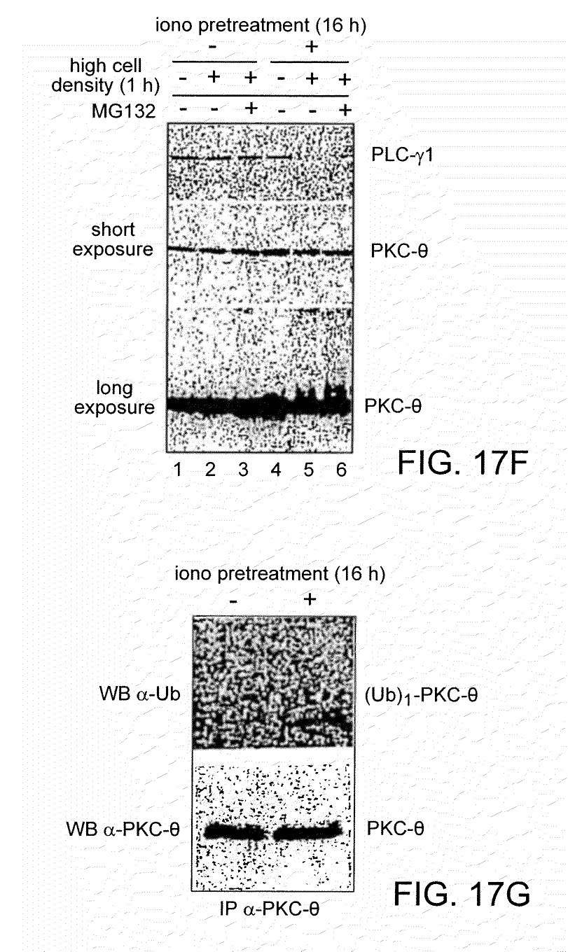

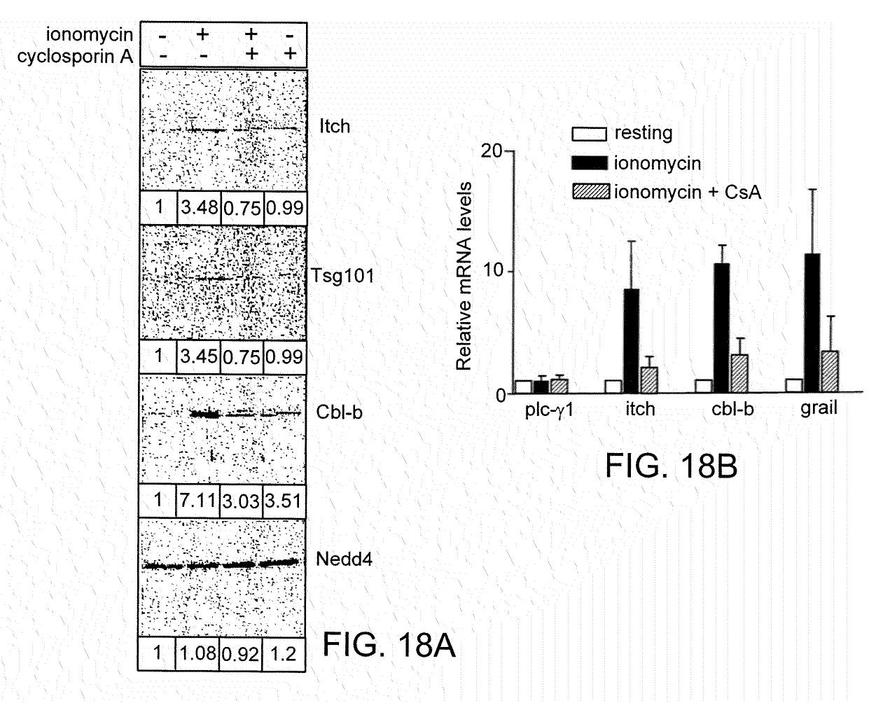

Intriguingly, all three targets of the Ca2+/calcineurin-dependent degradation program, PLC-γ1, PKCθ, and RasGAP, possess C2 domains (FIG. 17A) which mediate Ca2+-dependent phospholipid binding or promote protein-protein interactions that may or may not be Ca2+-dependent. C2 domains are also found in the Itch/Nedd4 family of E3 ubiquitin ligases (FIG. 17A). Whether these E3 ligases were involved in PLC-γ1 degradation was investigated. PLC-γ1 co-immunoprecipitated with both Nedd4 and Itch (FIG. 17B) and was a substrate for ubiquitination by Itch (FIG. 17C). In 293 cells, ionomycin treatment induced PLC-γ1 ubiquitination (FIG. 17C, lanes 4, 5), and a substantial fraction of the ubiquitinated PLC-γ1 migrated as a doublet corresponding to mono- and di-ubiquitinated forms (arrows, upper two panels of FIG. 17C). Co-expression of Itch strongly enhanced PLC-γ1 ubiquitination, increasing the levels of mono-, di- and poly-ubiquitinated forms (FIG. 17C, lanes 2, 3); however the ionomycin dependence of ubiquitination was less striking under these overexpression conditions. Itch and Nedd4 both facilitated the ionomycin-dependent degradation of PLC-γ1 (FIG. 17D, top panel, lanes 3, 4 and 7, 8); the decrease was best observed at low levels of Itch/Nedd4 expression (<2-4 fold overexpression compared to endogenous protein levels; see lower panel of FIG. 17D). A catalytically inactive Nedd4 protein, bearing an alanine substitution at the active cysteine of the HECT domain, did not promote this decrease (FIG. 17D; lanes 5, 6), but prevented the small but significant decrease in PLC-γ1 levels observed in ionomycin-treated cells (compare lanes 1, 2 and 5, 6 of FIG. 17D). Furthermore, sustained Ca2+ signaling followed by homotypic cell adhesion altered the subcellular localization of Itch and Nedd4 proteins in anergic T cells, causing a strong translocation of both proteins to the detergent-insoluble membrane fraction (FIG. 17E, top two panels). Under the same conditions, the membrane adapter LAT localized to both detergent-soluble and -insoluble membrane fractions and was equally abundant in these fractions in resting and anergized cells (bottom panel of FIG. 17E). In untreated T cells, Nedd4 was depleted from the cytosolic fraction and translocated to the detergent-insoluble fraction only in response to combined stimulation with anti-CD3 and anti-CD28 (FIG. 15B, top panels, lanes 1, 2 and 5, 6), whereas in ionomycin-pretreated cells, stimulation with anti-CD3 was sufficient for full membrane association of Nedd4 (FIG. 15B; lower panels, compare lanes 3, 5 with lanes 4, 6). Thus the C2-domain-containing E3 ligases Itch and Nedd4 are strong candidates for mediating PLC-γ1 degradation in T cells anergized by sustained Ca2+ signaling.

Surprisingly, the proteasome inhibitor MG132 did not prevent PLC-γ1 degradation (FIG. 17F), nor did it inhibit the decline of PKCθ levels observed in ionomycin-pretreated D5 T cells subjected to homotypic adhesion (data not shown). Rather, MG132 increased the accumulation, only in anergized T cells, of a modified form of PKCθ visible in a long exposure (FIG. 17F, compare lanes 1-3 with lanes 4-6). This species migrated with an apparent molecular weight ˜10 kDa greater than that of PKCθitself, suggesting that it represented a mono-ubiquitinated form. PKCθ mono-ubiquitination was demonstrated by immunoprecipitating PKCθ from untreated and anergized T cells, followed by Western blotting with antiubiquitin antibodies (FIG. 17G): untreated T cells showed no ubiquitination (lane 1) while ionomycin-pretreated T cells that were allowed to interact homotypically displayed a distinct band at a molecular weight corresponding to mono-ubiquitinated PKCθ, with no apparent signal at higher molecular weights (lane 2).

These results suggested that degradation of signaling proteins in anergic T cells was accomplished not via the proteasome, which binds with high affinity only to proteins tagged with 4 or more ubiquitin moieties, but rather via the lysosomal pathway, in which mono-ubiquitination promotes sorting of proteins associated with the limiting membrane of endosomes into small internal vesicles that accumulate in the lumen as the endosomes mature. In yeast, sorting is accomplished by the endosome-associated ESCRT-1 complex, which binds mono- and di-ubiquitin-tagged transmembrane proteins and sorts them into the invaginating structures that form the internal vesicles; the resulting multivesicular bodies fuse with lysosomes and deliver their contents for degradation. The critical ubiquitin-binding component of the yeast ESCRT-1 complex is Vps23p, the mammalian homologue of which is Tsg101. Tsg101 is essential for downregulation of the activated EGF-receptor, which is ubiquitinated by the E3 ligase Cbl. In T cells, Cbl proteins are known to diminish proximal TCR transduction by downregulating the TCR as well as by ubiquitinating and inducing degradation of TCR-coupled tyrosine kinases.

Whether Itch, Nedd4, Tsg101 and Cbl-b, the major Cbl family member in mature T cells, were upregulated in a Ca2+/calcineurin-dependent fashion during the priming step of anergy was investigated (FIG. 18A). Itch and Tsg101 protein levels increased ˜3-fold in ionomycin-treated D5 cells and the increase was blocked by CsA (FIG. 18A, top two panels). Cbl-b was even more highly induced and its induction was partly blocked by CsA (FIG. 18A; third panel). There was no change in Nedd4 protein levels under these conditions (FIG. 18A; bottom panel), despite the membrane relocalization of Nedd4 protein shown in FIGS. 17E and 15B. Itch protein levels also increased after “anergic” stimulation of D5 T cells with low concentrations of plate-bound anti-CD3, but not after productive activation with anti-CD3/anti-CD28 (FIG. 15C). Upregulation of the E3 ligases reflected an anergy-associated transcriptional program: PLC-γ1 mRNA levels remained constant, but the levels of mRNAs encoding Itch, Cbl-b and GRAIL (a novel anergy-associated E3 ligase) increased by 8 to 11-fold in ionomycin-treated T cells, and this increase was largely blocked by CsA (FIG. 18B). Furthermore, ectopic expression of constitutively-active NFAT which bore the “RIT” mutation that prevented interaction with AP-1 (Fos-Jun), was sufficient to upregulate Itch protein levels in NIH 3T3 cells (FIG. 15D), suggesting strongly that Itch is a target of the AP-1-independent NFAT transcriptional program that have been described previously.

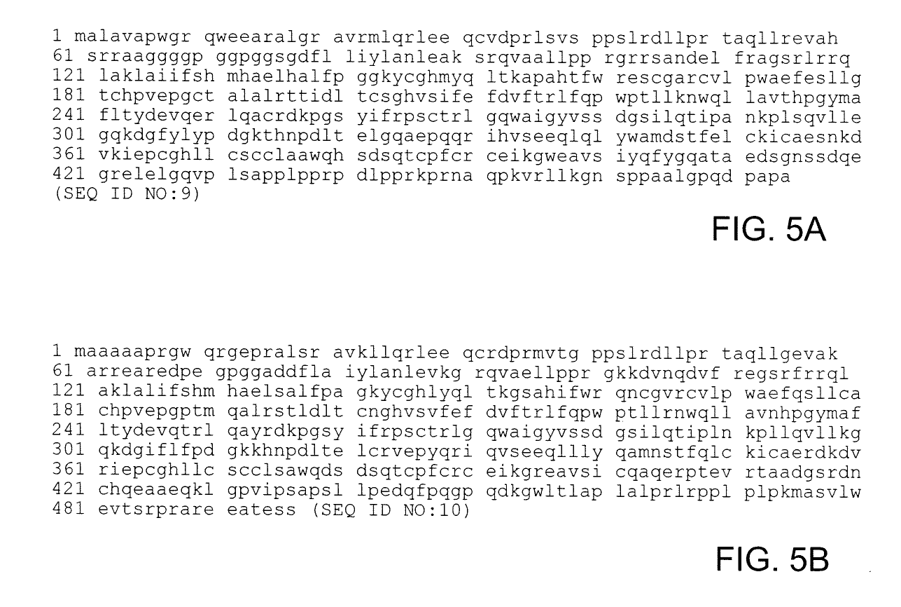

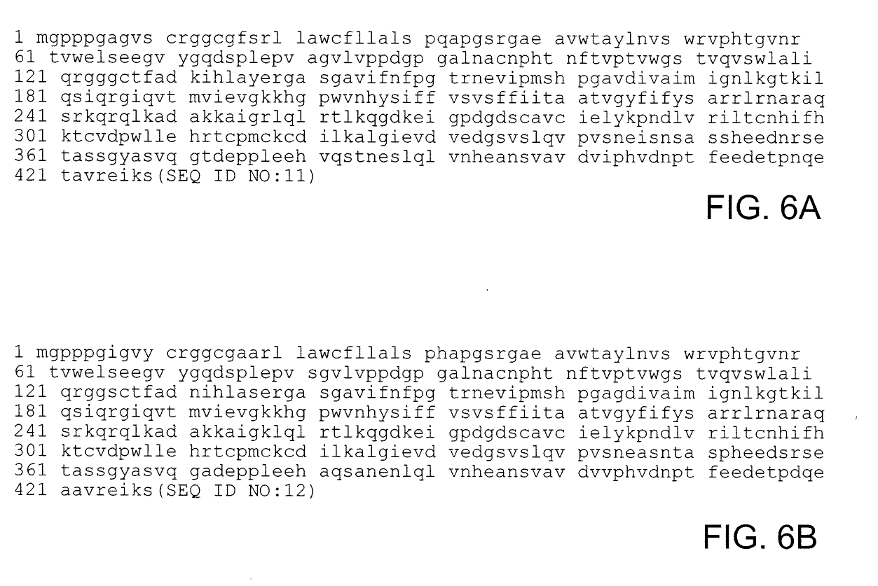

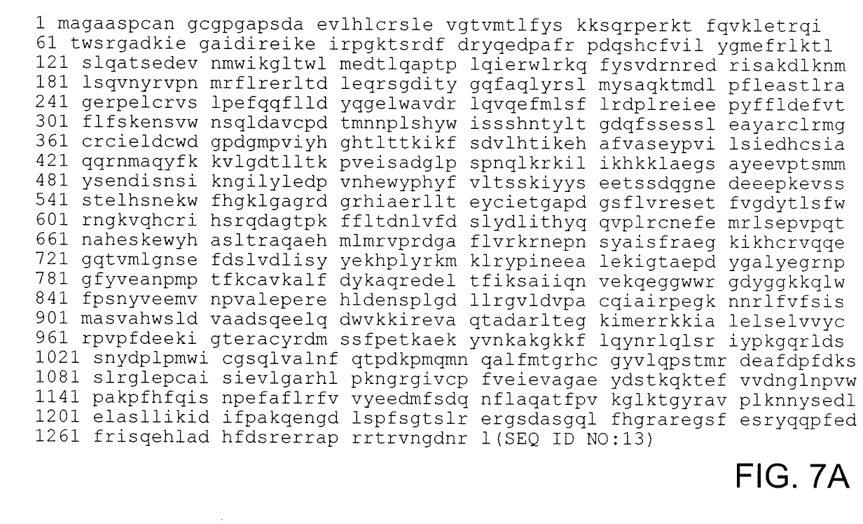

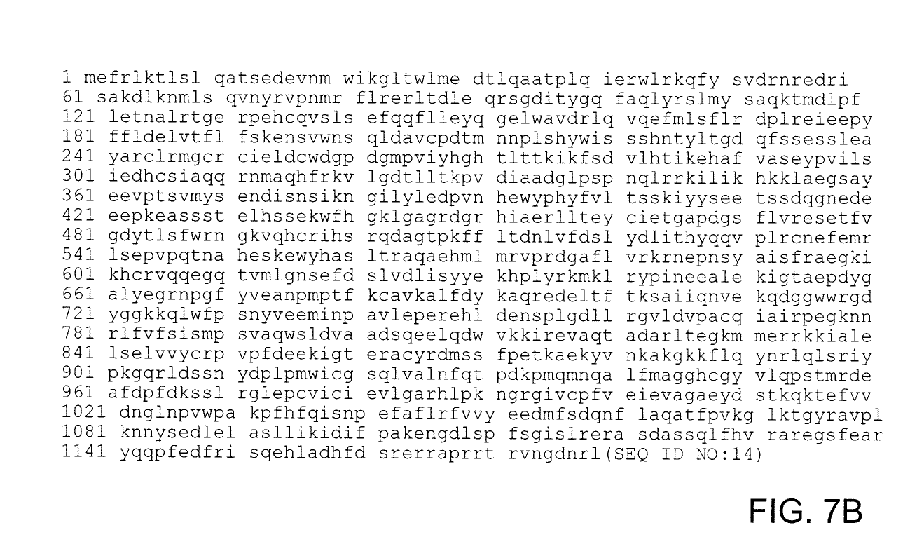

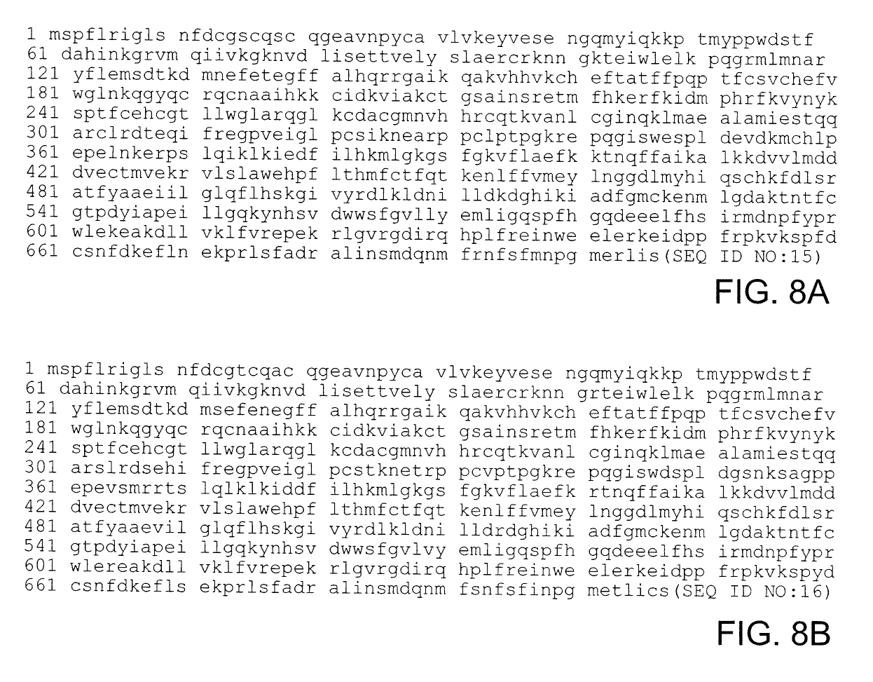

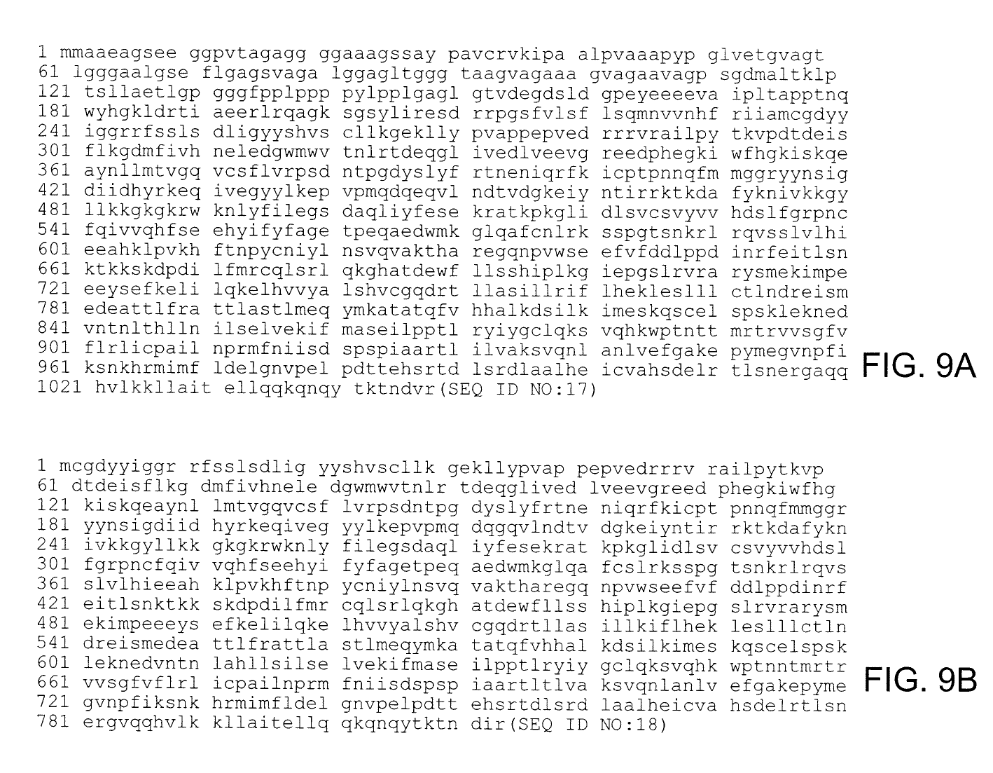

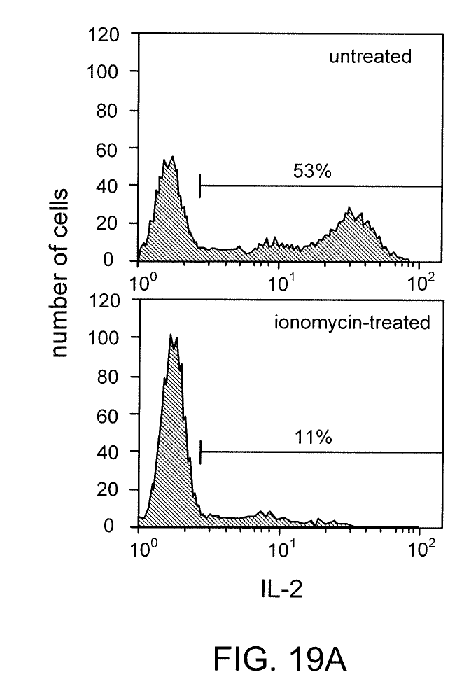

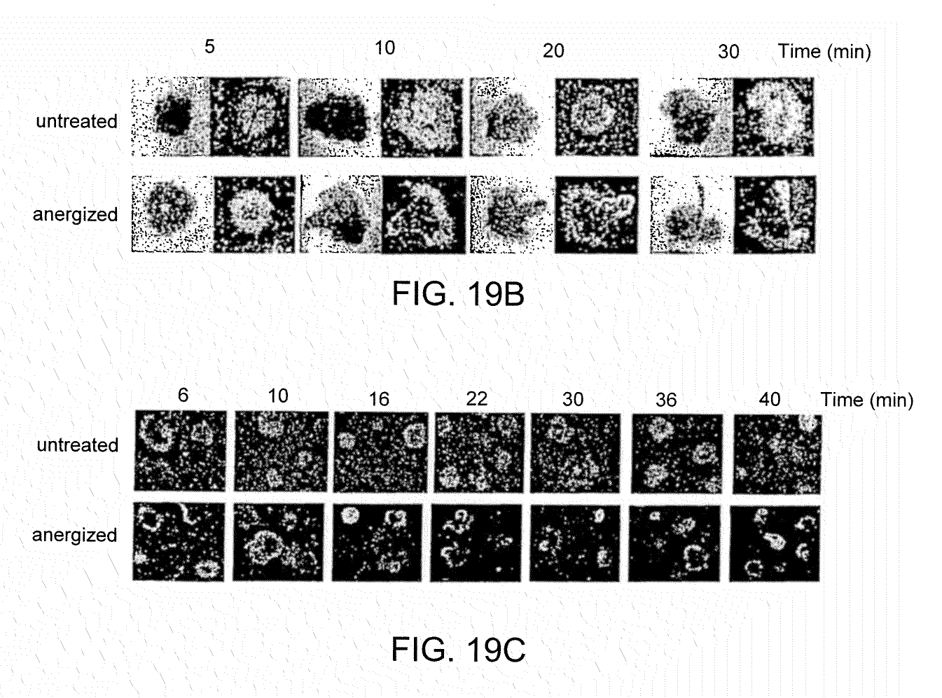

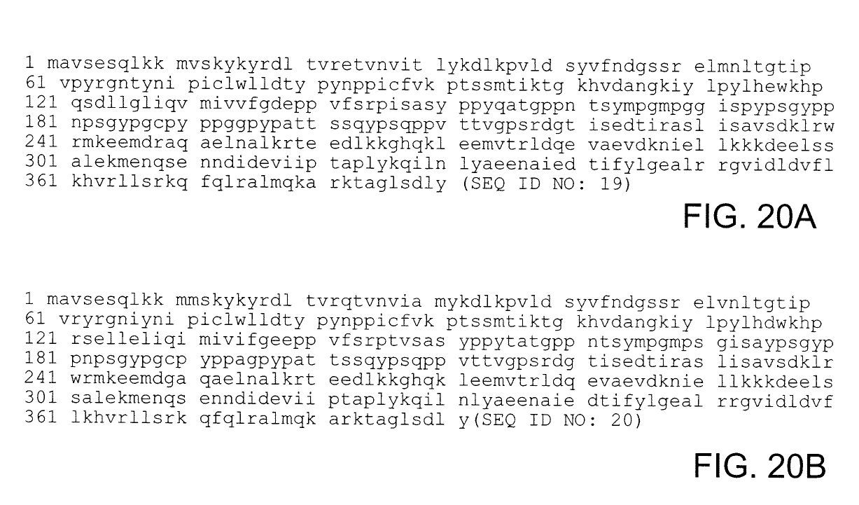

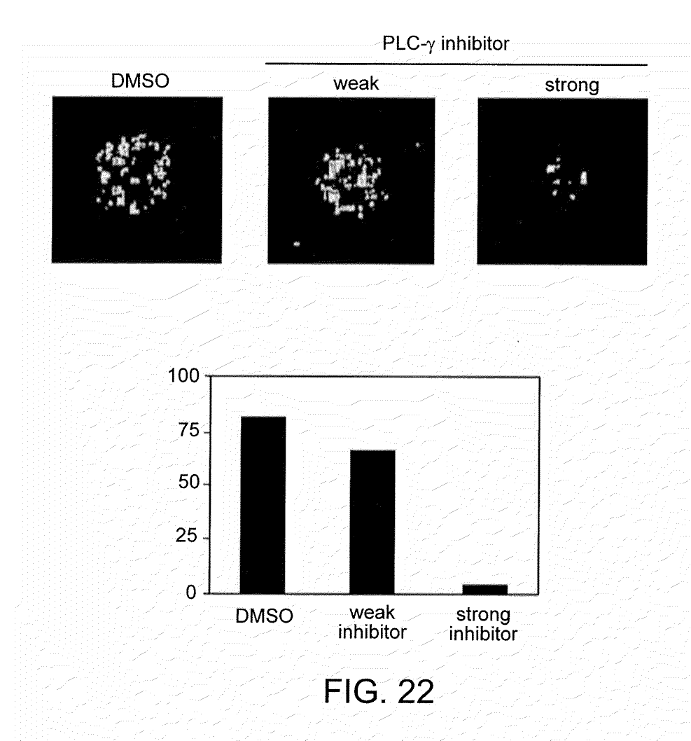

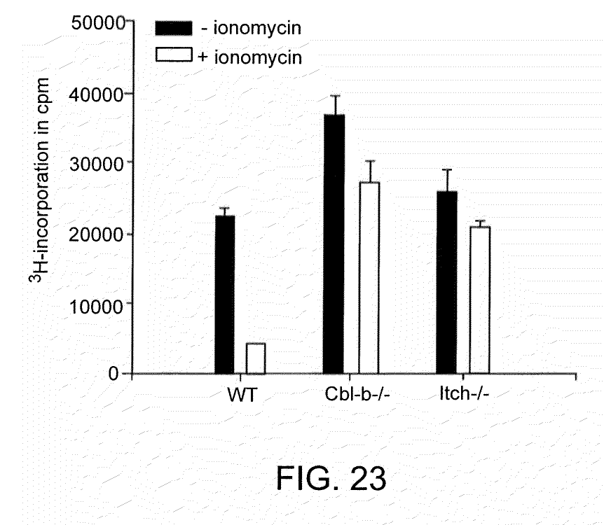

The interface (“immunological synapse”) between the T cell and the antigen-presenting cell (APC) is an important site for regulation of signaling. Formation of the immunological synapse in untreated and anergic T cells was monitored (FIGS. 19A-C). In both cases, the immature immunological synapse, characterized by peripheral TCR/MHC:peptide and central LFA-1/ICAM-1 contacts, developed quickly into the mature structure with a core TCR/MHC:peptide contact region and a peripheral LFA-1/ICAM-1 ring (FIGS. 19B and 19C, 5 and 6 min time points). The mature synapse persisted stably in the untreated T cells for at least an hour following initial contact; in contrast, anergic T cells showed partial or occasionally complete breakdown of the outer LFA-1 ring within 10-20 min after the mature synapse was established, and often also showed aberrant morphology of the inner TCR core (FIGS. 19B and 19C, 10 min and later). Parallel analysis of fluorescence and contact area patterns revealed that anergic T cells displayed a “migratory” phenotype, in which the LFA-1-ICAM-1 ring became disrupted and began to move away from the TCR-MHC clusters, which were dragged behind the moving T cells (FIG. 19B). To determine whether synapse instability was a direct consequence of the loss of PLC-γ1 function, T cells were allowed to establish mature synapses and then treated them with the strong phospholipase inhibitor U73122. This treatment evoked exactly the same phenotype of disintegration of the outer LFA-1 ring as observed in anergic T cells (FIG. 22). PKCθ has also been linked to efficient formation of the immunological synapse, since naïve PKCθ-deficient T cells are impaired in their ability to form synapses with dendritic cells, showing a reduced frequency of APC-T cell contact. Together, these data underscore the requirement for PLC-γ1 and PKCθ signaling in maintenance of the mature immunological synapse.

### Genetic Evidence for the Role of Itch and Cbl-b in the Induction of Anergy

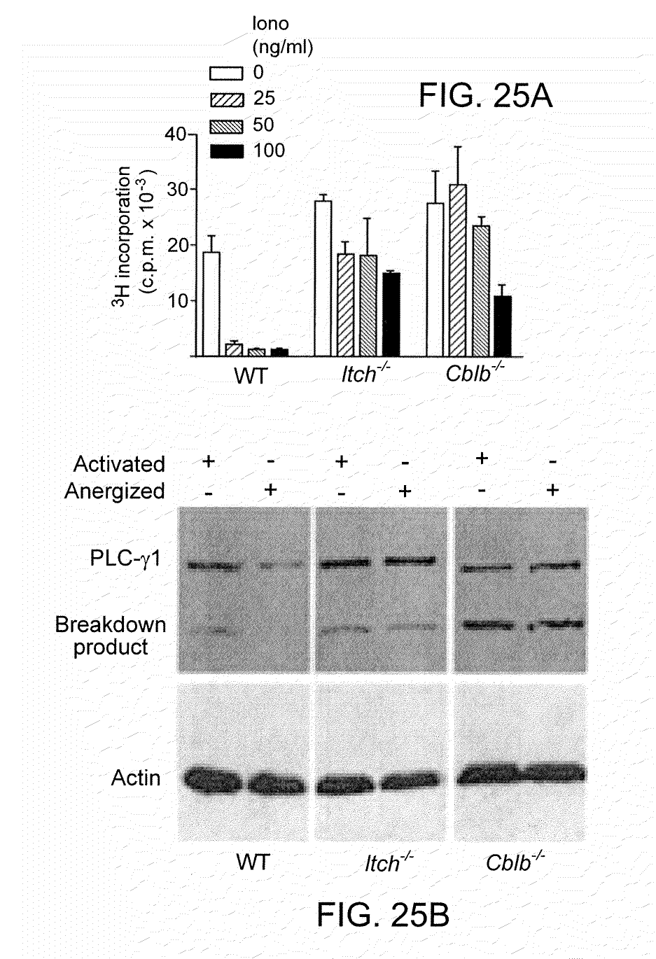

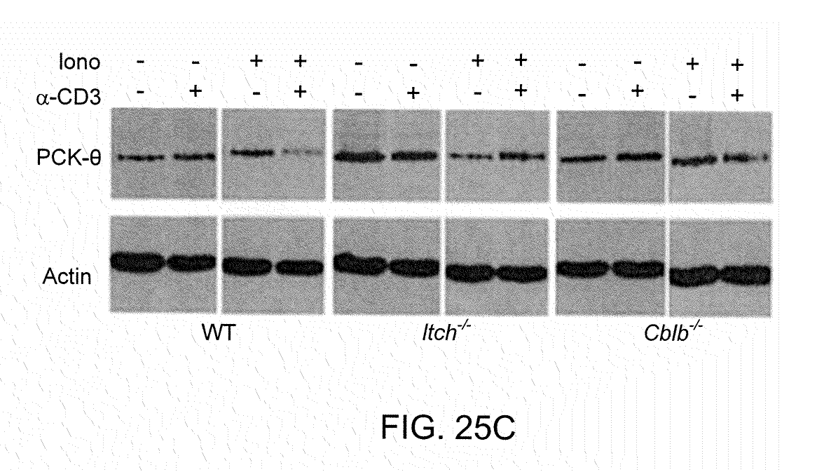

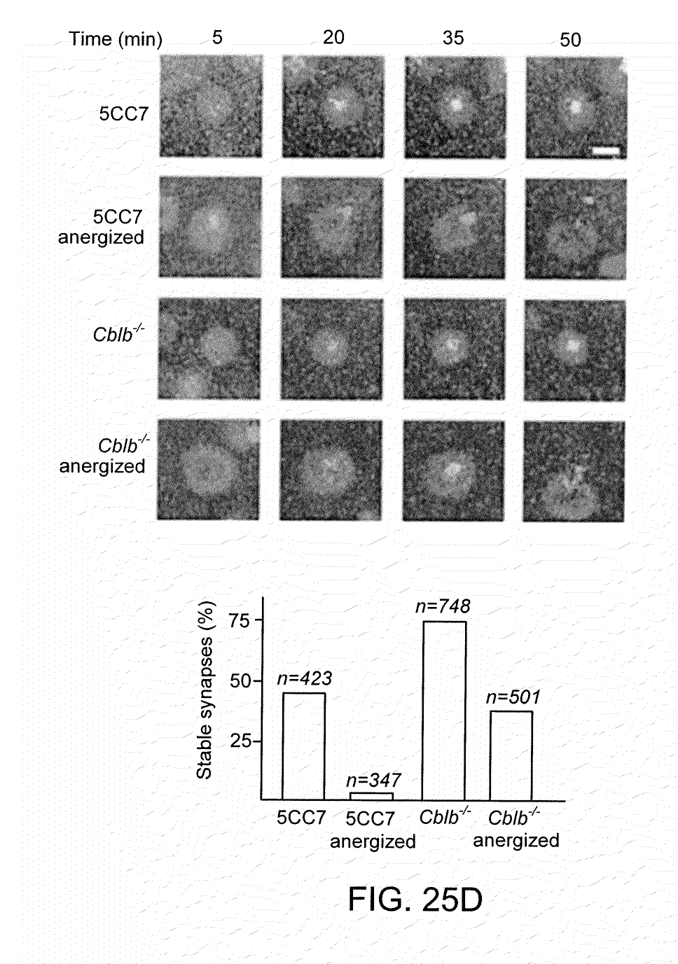

Mice deficient in either Itch or Cbl-b have autoimmune phenotypes (Fang et al. Nat. Immun. 3: 281-287 (2002) and Chiang et al., Nature 403:216-220 (2000), indicating that these E3 ligases are important in suppressing immune responses to self antigens. To evaluate the participation of Itch and Cbl-b in Ca²⁺-induced T cell anergy, we tested T cells from C57 BL/6 (WT), Itch^{-/-} (Itchy), and Cb/b^{-/-} mice. The results are shown in FIGS. 25A-D.

CD4 T cells from C57BL/6 (WT), Cblb−/− and Itch−/− mice were stimulated with anti-CD3 and anti-CD28 for 2 d and were left resting for 5 d. Cells were then left untreated or were treated for 16 h with 25-100 ng/ml of ionomycin (Iono), after which proliferative responses to anti-CD3 and anti-CD28 stimulation were measured by 3^{H} thymidine incorporation. FIG. 25A shows that Itch^{-/-} and Cb/b^{-/-} CD4 T cells were resistant to anergy induction at low doses of ionomycin, and this effect was partially overcome at higher doses of ionomycin.

The ability of Itch^{-/-} and Cb/b^{-/-} T cells to degrade PLC-γ1 and PKC-θ in response conditions that induce anergy in wild-type cells was assessed. TH1 cells from C57BL/6 (WT), Cblb−/− and Itch−/− mice were allowed to differentiate for 1 week, then were stimulated with plate-bound anti-CD3 in the presence of CTLA4-Ig (Anergized) or with anti-CD3 and anti-CD28 (Activated) for 2 d, then were allowed to ‘rest’ for 3 d in media without interleukin 2. Cell extracts were analyzed for PLC-1 and actin by immunoblotting, as shown in FIG. 25B. As expected, PLC-γ1 protein decreased in wild-type T cells after the cells were anergized with anti-CD3 stimulation in the absence of costimulation, but Itch^{-/-} and Cblb^{-/-} T cells did not show this decrease.

TH1 cells from C57BL/6 (WT), Itch−/− and Cblb−/− mice were left untreated (−) or were treated for 16 h with ionomycin (+), were washed, then were restimulated (+) or not (−) with plate-bound anti-CD3 (−CD3). Cell extracts were analyzed for PKC-θ and actin by immunoblotting, as shown in FIG. 25C. Wild-type T cells showed the expected decrease in PKC-θ protein after ionomycin pretreatment followed by restimulation with anti-CD3, but we did not find this effect in T cells from Itch^{-/-} and Cblb^{-/-} mice.

FIG. 25D shows a comparison of the kinetics of synapse disintegration in control and Cblb^{-/-} T cells that had been anergized by pretreatment with ionomycin. The formation of immune synapses was evaluated as described for the experiments shown in FIG. 19, with TH1 cells from wild-type or Cblb−/−5CC7 TCR-transgenic mice and lipid bilayers displaying ICAM-1 and I-Ek pigeon cytochrome C(PCC) molecules. Individual representative cells (genotypes, left margin) observed over a time course of 50 min are shown in the upper series of images. Below the image series is a histogram that quantifies the imaging results. The histogram shows the percentage of cells with stable synapses at 35 min after synapse formation was initiated. As expected, control 5CC7 TCR transgenic T cells exposed to peptide-loaded MHC and LFA-1 molecules in lipid bilayers formed synapses that were stable throughout the observation period of 50 min, whereas 5CC7 T cells that were pretreated with ionomycin for 16 h formed the mature synapse quickly (<5 min) on contact with the bilayer but then showed synapse disorganization and developed the migratory phenotype. Synapses formed by untreated Cblb^{-/-} T cells were as stable as those formed by wild-type T cells, but synapses formed by ionomycin-pretreated Cblb^{-/-} T cells were mostly protected from synapse disintegration, as judged by their stability for up to 35 min of observation. Thus, Cbl-b contributes substantially to the early disintegration of the immunological synapse in anergic T cells. However, the synapses break down at later times in ionomycin-pretreated Cb/b^{-/-} T cells (50 min), indicating that other factors are also involved.

These findings provide a plausible molecular mechanism for the autoimmune phenotypes of Cbl-b-deficient and Itchdeficient (Itchy) mice. Itchy mice display splenomegaly and lymphocyte infiltration in several tissues and chronic inflammation in the skin while cbl-b ablation is associated with spontaneous T cell activation and autoantibody production and enhanced experimental autoimmune encephalomyelitis (EAE); moreover, cbl-b is a major susceptibility gene for type I diabetes in rats.

The data appear to define a complex negative feedback program that implements T cell anergy. The program is initiated by Ca2+/calcineurin signaling and culminates in proteolytic degradation of several signaling proteins, among them PLC-γ1 and PKCθ, two central players in the TCR signaling cascade. The first step of the program requires sustained Ca2+/calcineurin signaling and results in upregulation of three E3 ligases Itch, Cbl-b and GRAIL, as well as the endosomal sorting receptor, Tsg101. As has been demonstrated for Itch, this upregulation is likely to be part of an AP-1-independent transcriptional program initiated by NFAT. Degradation is actually implemented during a second step of T cell-APC contact, during which the E3 ligases Itch, Nedd4 and Cbl-b move to detergent-insoluble membrane fractions where they may colocalize with activated substrate proteins. This membrane compartment may include endosomal membranes, consistent with previous findings that PLC-γ1, RasGAP, Tsg101 and GRAIL are all associated with endosomes. In the third step, it is possible that mono-ubiquitination of the signaling proteins promotes their stable interaction with proteins such as Tsg101 which contain ubiquitin-binding domains, resulting in their being sorted into multivesicular bodies and targeted for lysosomal degradation. The Nedd4/Itch family, Cbl proteins and Tsg101 are implicated in receptor endocytosis and lysosomal degradation in other systems; moreover there is considerable evidence that Nedd4 and Cbl proteins participate in the internalization process itself. The E3 ligase GRAIL, which resides in the endosomal membrane and is upregulated in anergic T cells, could synergize with these effectors to further enhance protein ubiquitination and degradation.

The genetic evidence indicates that both classes of E3 ligases, the Nedd4/Itch and Cbl/Cbl-b families, cooperate to induce T cell anergy. It is likely that Cbl proteins are needed to internalize the TCR, and that Itch and possibly GRAIL ubiquitinate receptor-associated proteins at the endosomal membrane. This process would be expected to occur mainly during the early stage of TCR activation when the immunological synapse matures and TCR internalization occurs. The attractive feature of this downregulatory program is that signaling molecules would be targets for degradation only when they are activated. In a normally-activated T cell, PLC-γ1-dependent production of second messengers will continue until PLC-Y1 is dephosphorylated or its substrate becomes limiting. In an anergic T cell in which the Itch, Cbl-b, Nedd4 and GRAIL E3 ligases are upregulated and/or preactivated for membrane localization, PLC-γ1 and PKCθ activation coincides with E3-mediated mono-ubiquitination which immediately, via Tsg101, would sequester the active enzymes within endosomes where it cannot be reactivated. Thus, anergy does not require massive depletion of cellular PLC-γ1; only the active PLC-γ1 signaling complexes coming to the membrane are rapidly eliminated. Consistent with this hypothesis, anergic T cells showed no appreciable downregulation of PLC-γ2, which has the same domain organization as PLC-γ1 but is not critical for T cell signaling.

The T cell anergy program resembles neuronal long-term depression, in which Ca2+/calcineurin signals downregulate synaptic activity and establish a hypo-responsive state. In T cells, anergy is imposed by the calcineurin-regulated transcription factor NFAT, while in neurons, LTD is mediated in part through acute changes in signaling that do not involve transcription. Recent evidence suggests that in Aplysia, synaptic plasticity related to long-term memory is associated with transcriptional and chromatin changes in the promoter regions of relevant genes. Notably, both neuronal and immune cells process information via close (“synaptic”) contacts with other cells, and both need to retain a memory of their previous cellular and environmental experience.

A number of embodiments of the invention have been described. Nevertheless, it will be understood that various modifications may be made without departing from the spirit and scope of the invention. Accordingly, other embodiments are within the scope of the following claims.

1. A method of identifying an anergy modulating agent, comprising:
(a) providing an E3 ubiquitin ligase polypeptide, E3 ubiquitin ligase substrate polypeptide, and a test compound;
(b) contacting the test compound, the ligase polypeptide, and the ligase substrate polypeptide together under conditions that allow the ligase polypeptide to bind or ubiquitinate the substrate polypeptide; and
(c) determining whether the test compound decreases the level of binding or ubiquitination of the substrate polypeptide by the ligase polypeptide, relative to the level in the absence of the test compound, wherein a decrease indicates that the test compound is an anergy modulating agent.

2. The method of claim 1, wherein the ligase polypeptide is selected from the group consisting of Itch, GRAIL, Cbl, Cbl-b, Cbl-b3, Aip4, and Nedd4.

3. The method of claim 1, wherein the ligase polypeptide comprises an amino acid sequence selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

4. The method of claim 1, wherein the substrate polypeptide is selected from the group consisting of: PLC-γ, PKCθ, and RasGAP.

5. The method of claim 1, wherein the substrate polypeptide comprises an amino acid sequence selected from the group consisting of: SEQ ID NO:13, SEQ ID NO:14, SEQ ID NO:15, SEQ ID NO:16, SEQ ID NO:17, and SEQ ID NO:18.

6. The method of claim 1, further comprising: (d) determining whether the agent reduces anergy in an immune cell in vivo or in vitro.

7-9. (canceled)

10. The method of claim 1, wherein the ligase polypeptide is Itch and the substrate polypeptide is PLC-γ.

11. The method of claim 1, wherein the ligase polypeptide is Itch and the substrate polypeptide is PKCθ.

12. The method of claim 1, wherein the ligase polypeptide is Aip4 and the substrate polypeptide is PLC-γ.

13. The method of claim 1, wherein the ligase polypeptide is Aip4 and the substrate polypeptide is PKCθ.

14-16. (canceled)

17. A method of identifying an anergy modulating agent, comprising:
(a) providing a test compound and a polypeptide selected from the group consisting of: Itch, Aip4, GRAIL, Cbl, Cbl-b, Cbl-b3, Nedd4, PLC-γ and PLCθ, or a biologically active fragment thereof;
(b) contacting the test compound and the polypeptide or fragment thereof under conditions that allow the test compound to bind the polypeptide or fragment thereof;
(c) determining whether the test compound binds the polypeptide or fragment thereof; and
(d) determining whether the test compound reduces anergy in an immune cell in vivo or in vitro, wherein a test compound that reduces anergy is an anergy modulating agent.

18-19. (canceled)

20. A method of identifying an anergy modulating agent, comprising:
(a) providing a test compound and a polypeptide comprising Itch, Aip4, or a HECT fragment of Itch or Aip4;
(b) contacting the test compound and the polypeptide under conditions that allow the test compound to interact with the polypeptide;
(c) contacting the polypeptide with a reaction mix comprising E1, E2, tagged ubiquitin, and ATP; and
(d) determining whether the test compound prevents the autoubiquitination of the polypeptide in the presence of the reaction mix; wherein a test compound that prevents the autoubiquitination of the polypeptide is an anergy modulating agent.

21. The method of claim 20, further comprising: (e) determining whether the agent reduces anergy in an immune cell in vivo or in vitro.

22. (canceled)

23. The method of claim 20, wherein the E2 is UbCH7.

24-28. (canceled)

29. A method of identifying an anergy modulating agent, the method comprising:
(a) contacting a test compound and an E3 ubiquitin ligase polypeptide under conditions that allow the test compound to interact with the ligase polypeptide;
(b) contacting the ligase polypeptide with a reaction mix comprising E1, E2, tagged ubiquitin, ATP, and an E3 ubiquitin ligase substrate polypeptide; and
(c) determining whether the test compound inhibits the ligase polypeptide from transubiquitinating the substrate polypeptide in the presence of the reaction mix, wherein a test compound that inhibits transubiquitination is an anergy modulating agent.

30. The method of claim 29, wherein the E2 is UbCH7.

31. The method of claim 29, further comprising: (d) determining whether the agent reduces anergy in an immune cell in vivo or in vitro.

32-38. (canceled)

39. A method of identifying an agent that inhibits protein-protein interaction between an anergy associated E3 ubiquitin ligase and an E3 ubiquitin ligase substrate, the method comprising:
(a) providing an E3 ubiquitin ligase polypeptide, E3 ubiquitin ligase substrate polypeptide, and a test compound, wherein the ligase polypeptide or the substrate polypeptide is labeled;
(b) contacting the ligase polypeptide, the substrate polypeptide, and the test compound, with each other; and
(c) determining the amount of label bound to the unlabeled polypeptide, wherein a reduction in the amount of label that binds the unlabeled polypeptide indicates that the test compound is an agent that inhibits protein-protein interaction between an anergy associated E3 ubiquitin ligase and an E3 ubiquitin ligase substrate.

40. A method of identifying an agent that inhibits protein-protein interaction between an anergy associated E3 ubiquitin ligase and an E2 ubiquitin ligase, the method comprising:
(a) providing E3 ubiquitin ligase polypeptide, E2 ubiquitin ligase polypeptide, and a test compound, wherein the E3 ligase polypeptide or the E2 ubiquitin ligase polypeptide is labeled;
(b) contacting E3 ubiquitin ligase polypeptide, the E2 ubiquitin ligase polypeptide, and the test compound with each other; and
(c) determining the amount of label bound to the unlabeled ligase polypeptide, wherein a reduction in the amount of label that binds the unlabeled ligase indicates that the test compound is an agent that inhibits protein-protein interaction between an anergy associated E3 ubiquitin ligase and an E2 ubiquitin ligase.

41-54. (canceled)


SEQUENCE LISTING (31 sequences)

SEQ ID NO: 1 - protein, 862 aa, Homo sapiens
MSDSGSQLGSMGSLTMKSQLQITVISAKLKENKKNWFGPSPYVEVTVDGQSKKTEKCNNTNSPKWKQPLTVIVTPVSKLHFRVWSHQTLKSDVLLGTAALDIYETLKSNNMKLEEVVVTLQLGGDKEPTETIGDLSICLDGLQLESEVVTNGETTCSESASQNDDGSRSKDETRVSTNGSDDPEDAGAGENRRVSGNNSPSLSNGGFKPSRPPRPSRPPPPTPRRPASVNGSPSATSESDGSSTGSLPPTNTNTNTSEGATSGLIIPLTISGGSGPRPLNPVTQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECDLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYARTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 2 - protein, 854 aa, Mus musculus
MGSLTMKSQLQITVISAKLKENKKNWFGPSPYVEVTVDGQSKKTEKCNNTNSPKWKQPLTVIVTPTSKLCFRVWSHQTLKSDVLLGTAGLDIYETLKSNNMKLEEVVMTLQLVGDKEPTETMGDLSVCLDGLQVEAEVVTNGETSCSESTTQNDDGCRTRDDTRVSTNGSEDPEVAASGENKRANGNNSPSLSNGGFKPSRPPRPSRPPPPTPRRPASVNGSPSTNSDSDGSSTGSLPPTNTNVNTSTSEGATSGLIIPLTISGGSGPRPLNTVSQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQNKEFDPLGPLPPGWEKRTDSNGRVYFVNHNTRITQWEDPRSQGQLNEKPLPEGWEMRFTVDGIPYFVDHNRRATTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECGLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYTRTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 3 - protein, 900 aa, Homo sapiens
MATCAVEVFGLLEDEENSRIVRVRVIAGIGLAKKDILGASDPYVRVTLYDPMNGVLTSVQTKTIKKSLNPKWNEEILFRVHPQQHRLLFEVFDENRLTRDDFLGQVDVPLYPLPTENPRLERPYTFKDFVLHPRSHKSRVKGYLRLKMTYLPKTSGSEDDNAEQAEELEPGWVVLDQPDAACHLQQQQEPSPLPPGWEERQDILGRTYYVNHESRRTQWKRPTPQDNLTDAENGNIQLQAQRAFTTRRQISEETESVDNRESSENWEIIREDEATMYSNQAFPSPPPSSNLDVPTHLAEELNARLTIFGNSAVSQPASSSNHSSRRGSLQAYTFEEQPTLPVLLPTSSGLPPGWEEKQDERGRSYYVDHNSRTTTWTKPTVQATVETSQLTSSQSSAGPQSQASTSDSGQQVTQPSEIEQGFLPKGWEVRHAPNGRPFFIDHNTKTTTWEDPRLKIPAHLRGKTSLDTSNDLGPLPPGWEERTHTDGRIFYINHNIKRTQWEDPRLENVAITGPAVPYSRDYKRKYEFFRRKLKKQNDIPNKFEMKLRRATVLEDSYRRIMGVKRADFLKARLWIEFDGEKGLDYGGVAREWFFLISKEMFNPYYGLFEYSATDNYTLQINPNSGLCNEDHLSYFKFIGRVAGMAVYHGKLLDGFFIRPFYKMMLHKPITLHDMESVDSEYYNSLRWILENDPTELDLRFIIDEELFGQTHQHELKNGGSEIVVTNKNKKEYIYLVIQWRFVNRIQKQMAAFKEGFFELIPQDLIKIFDENELELLMCGLGDVDVNDWREHTKYKNGYSANHQVIQWFWKAVLMMDSEKRIRLLQFVTGTSRVPMNGFAELYGSNGPQSFTVEQWGTPEKLPRAHTCFNRLDLPPYESFEELWDKLQMAIENTQGFDGVD

SEQ ID NO: 4 - protein, 777 aa, Mus musculus
MSGILTSVQTKTIKKSLNPKWNEEILFRVLPQRHRILFEVFDENRLTRDDFLGQVDVPLYPLPTENPRMERPYTFKDFVLHPRSHKSRVKGYLRLKMTYLPKNGSEDENADQAEELEPGWVVLDQPDAATHLPHPPEPSPLPPGWEERQDVLGRTYYVNHESRRTQWKRPSPDDDLTDEDNDDMQLQAQRAFTTRRQISEDVDGPDNRESPENWEIVREDENTEYSGQAVQSPPSGHIDVQTHLAEEFNTRLAVCGNPATSQPVTSSNHSSRGGSLQTCIFEEQPTLPVLLPTSSGLPPGWEEKQDDRGRSYYVDHNSKTTTWSKPTMQDDPRSKIPAHLRGKTDSNDLGPLPPGWEERTHTDGRVFFINHNIKKTQWEDPRLQNVAITGPAVPYSRDYKRKYEFFRRKLKKQTDIPNKFEMKLRRANILEDSYRRIMGVKRADLLKARLWIEFDGEKGLDYGGVAREWFFLISKEMFNPYYGLFEYSATDNYTLQINPNSGLCNEDHLSYFKFIGRVAGMAVYHGKLLDGFFIRPFYKMMLQKLITLHDMESVDSEYYSSLRWILENDPTELDLRFIIDEELFGQTHQHELKTGGSEIVVTNKNKKEYIYLVIQWRFVNRIQKQMAAFKEGFFELIPQDLIKIFDENELELLMCGLGDVDVNDWREHTKYKNGYSMNHQVIHWFWKAVWMMDSEKRIRLLQFVTGTSRVPMNGFAELYGSNGPQSFTVEQWGTPDKLPRAHTCFNRLDLPPYESFDELWDKLQMAIENTQGFDGVD

SEQ ID NO: 5 - protein, 906 aa, Homo sapiens
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTYEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 6 - protein, 896 aa, Mus musculus
MAGNVKKSSGAGGGGSGGSGAGGLIGLMKDAFQPHHHHHHLSPHPPCTVDKKMVEKCWKLMDKVVRLCQNPNVALKNSPPYILDLLPDTYQHLRTVLSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQICAENDKDVKIEPCGHLMCTSCLTSWQESEGQGCPFCRCEIKGTEPIVVDPFDPRGSGSLLRQGAEGAPSPNYDDDDDERADDSLFMMKELAGAKVERPSSPFSMAPQASLPPVPPRLDLLQQRAPVPASTSVLGTASKAASGSLHKDKPLPIPPTLRDLPPPPPPDRPYSVGAETRPQRRPLPCTPGDCPSRDKLPPVPSSRPGDSWLSRTIPKVPVATPNPGDPWNGRELTNRHSLPFSLPSQMEPRADVPRLGSTFSLDTSMTMNSSPVAGPESEHPKIKPSSSANAIYSLAARPLPMPKLPPGEQGESEEDTEYMTPTSRPVGVQKPEPKRPLEATQSSRACDCDQQIDSCTYEAMYTIQSQALSVAENSASGEGNLATAHTSTGPEESENEDDGYDVPKPPVPAVLARRTLSDISNASSSFGWLSLDGDPTNFNEGSQVPERPPKPFPRRINSERKASSYQQGGGATANPVATAPSPQLSSEIERLMSQGYSYQDIQKALVIAHNNIEMAKNILREFVSISSPAHVAT

SEQ ID NO: 7 - protein, 982 aa, Homo sapiens [sequence omitted]

SEQ ID NO: 8 - protein, 547 aa, Mus musculus
MCTSCLTAWQESDGQGCPFCRCEIKGTEPIIVDPFDPRDEGSRCCSIIDPFSIPMLDLDDDDDREESLMMNRLASVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPERPPPIPPDNRLSRHFHHGESVPSRDQPMPLEAWCPRDAFGTNQVMGCRILGDGSPKPGVTANSSLNGRHSRMGSEQVLMRKHRRHDLPSEGAKVFSNGHLATEEYDVPPRLSPPPPVTTLLPSIKCTGPLANCLSEKTRDTVEDDDDEYKIPSSHPVSLNSQPSHCHNVKAPVRSCDNGHCILNGTHGAPSEMKKSNIPDLGIYLKGEDAFDALPPSLPPPPPPARHSLIEHSKPPGSSSRPSSGQDLFLLPSDPFFDPTSGQVPLPPARRAAGDSGKANRASQDYDQLPSSSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 9 - protein, 474 aa, Homo sapiens
MALAVAPWGRQWEEARALGRAVRMLQRLEEQCVDPRLSVSPPSLRDLLPRTAQLLREVAHSRRAAGGGGPGGPGGSGDFLLIYLANLEAKSRQVAALLPPRGRRSANDELFRAGSRLRRQLAKLAIIFSHMHAELHALFPGGKYCGHMYQLTKAPAHTFWRESCGARCVLPWAEFESLLGTCHPVEPGCTALALRTTIDLTCSGHVSIFEFDVFTRLFQPWPTLLKNWQLLAVTHPGYMAFLTYDEVQERLQACRDKPGSYIFRPSCTRLGQWAIGYVSSDGSILQTIPANKPLSQVLLEGQKDGFYLYPDGKTHNPDLTELGQAEPQQRIHVSEEQLQLYWAMDSTFELCKICAESNKDVKIEPCGHLLCSCCLAAWQHSDSQTCPFCRCEIKGWEAVSIYQFYGQATAEDSGNSSDQEGRELELGQVPLSAPPLPPRPDLPPRKPRNAQPKVRLLKGNSPPAALGPQDPAPA

SEQ ID NO: 10 - protein, 496 aa, Mus musculus
MAAAAAPRGWQRGEPRALSRAVKLLQRLEEQCRDPRMVTGPPSLRDLLPRTAQLLGEVAKARREAREDPEGPGGADDFLAIYLANLEVKGRQVAELLPPRGKKDVNQDVFREGSRFRRQLAKLALIFSHMHAELSALFPAGKYCGHLYQLTKGSAHIFWRQNCGVRCVLPWAEFQSLLCACHPVEPGPTMQALRSTLDLTCNGHVSVFEFDVFTRLFQPWPTLLRNWQLLAVNHPGYMAFLTYDEVQTRLQAYRDKPGSYIFRPSCTRLGQWAIGYVSSDGSILQTIPLNKPLLQVLLKGQKDGIFLFPDGKKHNPDLTELCRVEPYQRIQVSEEQLLLYQAMNSTFQLCKICAERDKDVRIEPCGHLLCSCCLSAWQDSDSQTCPFCRCEIKGREAVSICQAQERPTEVRTAADGSRDNCHQEAAEQKLGPVIPSAPSLLPEDQFPQGPQDKGWLTLAPLALPRLRPPLPLPKMASVLWEVTSRPRAREEATESS

SEQ ID NO: 11 - protein, 428 aa, Homo sapiens
MGPPPGAGVSCRGGCGFSRLLAWCFLLALSPQAPGSRGAEAVWTAYLNVSWRVPHTGVNRTVWELSEEGVYGQDSPLEPVAGVLVPPDGPGALNACNPHTNFTVPTVWGSTVQVSWLALIQRGGGCTFADKIHLAYERGASGAVIFNFPGTRNEVIPMSHPGAVDIVAIMIGNLKGTKILQSIQRGIQVTMVIEVGKKHGPWVNHYSIFFVSVSFFIITAATVGYFIFYSARRLRNARAQSRKQRQLKADAKKAIGRLQLRTLKQGDKEIGPDGDSCAVCIELYKPNDLVRILTCNHIFHKTCVDPWLLEHRTCPMCKCDILKALGIEVDVEDGSVSLQVPVSNEISNSASSHEEDNRSETASSGYASVQGTDEPPLEEHVQSTNESLQLVNHEANSVAVDVIPHVDNPTFEEDETPNQETAVREIKS

SEQ ID NO: 12 - protein, 428 aa, Mus musculus
MGPPPGIGVYCRGGCGAARLLAWCFLLALSPHAPGSRGAEAVWTAYLNVSWRVPHTGVNRTVWELSEEGVYGQDSPLEPVSGVLVPPDGPGALNACNPHTNFTVPTVWGSTVQVSWLALIQRGGSCTFADNIHLASERGASGAVIFNFPGTRNEVIPMSHPGAGDIVAIMIGNLKGTKILQSIQRGIQVTMVIEVGKKHGPWVNHYSIFFVSVSFFIITAATVGYFIFYSARRLRNARAQSRKQRQLKADAKKAIGKLQLRTLKQGDKEIGPDGDSCAVCIELYKPNDLVRILTCNHIFHKTCVDPWLLEHRTCPMCKCDILKALGIEVDVEDGSVSLQVPVSNEASNTASPHEEDSRSETASSGYASVQGADEPPLEEHAQSANENLQLVNHEANSVAVDVVPHVDNPTFEEDETPDQEAAVREIKS

SEQ ID NO: 13 - protein, 1291 aa, Homo sapiens
MAGAASPCANGCGPGAPSDAEVLHLCRSLEVGTVMTLFYSKKSQRPERKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPLQIERWLRKQFYSVDRNREDRISAKDLKNMLSQVNYRVPNMRFLRERLTDLEQRSGDITYGQFAQLYRSLMYSAQKTMDLPFLEASTLRAGERPELCRVSLPEFQQFLLDYQGELWAVDRLQVQEFMLSFLRDPLREIEEPYFFLDEFVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQYFKKVLGDTLLTKPVEISADGLPSPNQLKRKILIKHKKLAEGSAYEEVPTSMMYSENDISNSIKNGILYLEDPVNHEWYPHYFVLTSSKIYYSEETSSDQGNEDEEEPKEVSSSTELHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFIKSAIIQNVEKQEGGWWRGDYGGKKQLWFPSNYVEEMVNPVALEPEREHLDENSPLGDLLRGVLDVPACQIAIRPEGKNNRLFVFSISMASVAHWSLDVAADSQEELQDWVKKIREVAQTADARLTEGKIMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMTGRHCGYVLQPSTMRDEAFDPFDKSSLRGLEPCAISIEVLGARHLPKNGRGIVCPFVEIEVAGAEYDSTKQKTEFVVDNGLNPVWPAKPFHFQISNPEFAFLRFVVYEEDMFSDQNFLAQATFPVKGLKTGYRAVPLKNNYSEDLELASLLIKIDIFPAKQENGDLSPFSGTSLRERGSDASGQLFHGRAREGSFESRYQQPFEDFRISQEHLADHFDSRERRAPRRTRVNGDNRL

SEQ ID NO: 14 - protein, 1178 aa, Mus musculus
MEFRLKTLSLQATSEDEVNMWIKGLTWLMEDTLQAATPLQIERWLRKQFYSVDRNREDRISAKDLKNMLSQVNYRVPNMRFLRERLTDLEQRSGDITYGQFAQLYRSLMYSAQKTMDLPFLETNALRTGERPEHCQVSLSEFQQFLLEYQGELWAVDRLQVQEFMLSFLRDPLREIEEPYFFLDELVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQHFRKVLGDTLLTKPVDIAADGLPSPNQLRRKILIKHKKLAEGSAYEEVPTSVMYSENDISNSIKNGILYLEDPVNHEWYPHYFVLTSSKIYYSEETSSDQGNEDEEEPKEASSSTELHSSEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFTKSAIIQNVEKQDGGWWRGDYGGKKQLWFPSNYVEEMINPAVLEPEREHLDENSPLGDLLRGVLDVPACQIAIRPEGKNNRLFVFSISMPSVAQWSLDVAADSQEELQDWVKKIREVAQTADARLTEGKMMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMAGGHCGYVLQPSTMRDEAFDPFDKSSLRGLEPCVICIEVLGARHLPKNGRGIVCPFVEIEVAGAEYDSTKQKTEFVVDNGLNPVWPAKPFHFQISNPEFAFLRFVVYEEDMFSDQNFLAQATFPVKGLKTGYRAVPLKNNYSEDLELASLLIKIDIFPAKENGDLSPFSGISLRERASDASSQLFHVRAREGSFEARYQQPFEDFRISQEHLADHFDSRERRAPRRTRVNGDNRL

SEQ ID NO: 15 - protein, 706 aa, Homo sapiens
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 16 - protein, 707 aa, Mus musculus
MSPFLRIGLSNFDCGTCQACQGEAVNPYCAVLVKEYVESENGQMYIQKKPTMYPPWDSTFDAHINKGRVMQIIVKGKNVDLISETTVELYSLAERCRKNNGRTEIWLELKPQGRMLMNARYFLEMSDTKDMSEFENEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARSLRDSEHIFREGPVEIGLPCSTKNETRPPCVPTPGKREPQGISWDSPLDGSNKSAGPPEPEVSMRRTSLQLKLKIDDFILHKMLGKGSFGKVFLAEFKRTNQFFAIKALKKDVVLMDDDVECTMVEKRVLSLAWEHPFLTHMFCTFQTKENLFFVMEYLNGGDLMYHIQSCHKFDLSRATFYAAEVILGLQFLHSKGIVYRDLKLDNILLDRDGHIKIADFGMCKENMLGDAKTNTFCGTPDYIAPEILLGQKYNHSVDWWSFGVLVYEMLIGQSPFHGQDEEELFHSIRMDNPFYPRWLEREAKDLLVKLFVREPEKRLGVRGDIRQHPLFREINWEELERKEIDPPFRPKVKSPYDCSNFDKEFLSEKPRLSFADRALINSMDQNMFSNFSFINPGMETLICS

SEQ ID NO: 17 - protein, 1047 aa, Homo sapiens
MMAAEAGSEEGGPVTAGAGGGGAAAGSSAYPAVCRVKIPAALPVAAAPYPGLVETGVAGTLGGGAALGSEFLGAGSVAGALGGAGLTGGGTAAGVAGAAAGVAGAAVAGPSGDMALTKLPTSLLAETLGPGGGFPPLPPPPYLPPLGAGLGTVDEGDSLDGPEYEEEEVAIPLTAPPTNQWYHGKLDRTIAEERLRQAGKSGSYLIRESDRRPGSFVLSFLSQMNVVNHFRIIAMCGDYYIGGRRFSSLSDLIGYYSHVSCLLKGEKLLYPVAPPEPVEDRRRVRAILPYTKVPDTDEISFLKGDMFIVHNELEDGWMWVTNLRTDEQGLIVEDLVEEVGREEDPHEGKIWFHGKISKQEAYNLLMTVGQVCSFLVRPSDNTPGDYSLYFRTNENIQRFKICPTPNNQFMMGGRYYNSIGDIIDHYRKEQIVEGYYLKEPVPMQDQEQVLNDTVDGKEIYNTIRRKTKDAFYKNIVKKGYLLKKGKGKRWKNLYFILEGSDAQLIYFESEKRATKPKGLIDLSVCSVYVVHDSLFGRPNCFQIVVQHFSEEHYIFYFAGETPEQAEDWMKGLQAFCNLRKSSPGTSNKRLRQVSSLVLHIEEAHKLPVKHFTNPYCNIYLNSVQVAKTHAREGQNPVWSEEFVFDDLPPDINRFEITLSNKTKKSKDPDILFMRCQLSRLQKGHATDEWFLLSSHIPLKGIEPGSLRVRARYSMEKIMPEEEYSEFKELILQKELHVVYALSHVCGQDRTLLASILLRIFLHEKLESLLLCTLNDREISMEDEATTLFRATTLASTLMEQYMKATATQFVHHALKDSILKIMESKQSCELSPSKLEKNEDVNTNLTHLLNILSELVEKIFMASEILPPTLRYIYGCLQKSVQHKWPTNTTMRTRVVSGFVFLRLICPAILNPRMFNIISDSPSPIAARTLILVAKSVQNLANLVEFGAKEPYMEGVNPFIKSNKHRMIMFLDELGNVPELPDTTEHSRTDLSRDLAALHEICVAHSDELRTLSNERGAQQHVLKKLLAITELLQQKQNQYTKTNDVR

SEQ ID NO: 18 - protein, 813 aa, Mus musculus
MCGDYYIGGRRFSSLSDLIGYYSHVSCLLKGEKLLYPVAPPEPVEDRRRVRAILPYTKVPDTDEISFLKGDMFIVHNELEDGWMWVTNLRTDEQGLIVEDLVEEVGREEDPHEGKIWFHGKISKQEAYNLLMTVGQVCSFLVRPSDNTPGDYSLYFRTNENIQRFKICPTPNNQFMMGGRYYNSIGDIIDHYRKEQIVEGYYLKEPVPMQDQGQVLNDTVDGKEIYNTIRRKTKDAFYKNIVKKGYLLKKGKGKRWKNLYFILEGSDAQLIYFESEKRATKPKGLIDLSVCSVYVVHDSLFGRPNCFQIVVQHFSEEHYIFYFAGETPEQAEDWMKGLQAFCSLRKSSPGTSNKRLRQVSSLVLHIEEAHKLPVKHFTNPYCNIYLNSVQVAKTHAREGQNPVWSEEFVFDDLPPDINRFEITLSNKTKKSKDPDILFMRCQLSRLQKGHATDEWFLLSSHIPLKGIEPGSLRVRARYSMEKIMPEEEYSEFKELILQKELHVVYALSHVCGQDRTLLASILLKIFLHEKLESLLLCTLNDREISMEDEATTLFRATTLASTLMEQYMKATATQFVHHALKDSILKIMESKQSCELSPSKLEKNEDVNTNLAHLLSILSELVEKIFMASEILPPTLRYIYGCLQKSVQHKWPTNNTMRTRVVSGFVFLRLICPAILNPRMFNIISDSPSPIAARTLTLVAKSVQNLANLVEFGAKEPYMEGVNPFIKSNKHRMIMFLDELGNVPELPDTTEHSRTDLSRDLAALHEICVAHSDELRTLSNERGVQQHVLKKLLAITELLQQKQNQYTKTNDIR

SEQ ID NO: 19 - protein, 390 aa, Homo sapiens
MAVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRPISASYPPYQATGPPNTSYMPGMPGGISPYPSGYPPNPSGYPGCPYPPGGPYPATTSSQYPSQPPVTTVGPSRDGTISEDTIRASLISAVSDKLRWRMKEEMDRAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 20 - protein, 391 aa, Mus musculus
MAVSESQLKKMMSKYKYRDLTVRQTVNVIAMYKDLKPVLDSYVFNDGSSRELVNLTGTIPVRYRGNIYNIPICLWLLDTYPYNPPICFVKPTSSMTIKTGKHVDANGKIYLPYLHDWKHPRSELLELIQIMIVIFGEEPPVFSRPTVSASYPPYTATGPPNTSYMPGMPSGISAYPSGYPPNPSGYPGCPYPPAGPYPATTSSQYPSQPPVTTVGPSRDGTISEDTIRASLISAVSDKLRWRMKEEMDGAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 21 - DNA, 20 bp, Artificial Sequence
  Primer
caagagggagagcaagccta

SEQ ID NO: 22 - DNA, 20 bp, Artificial Sequence
  Primer
aagcctttgacccctttgat

SEQ ID NO: 23 - DNA, 20 bp, Artificial Sequence
  Primer
ggttcagtccgttgtccact

SEQ ID NO: 24 - DNA, 20 bp, Artificial Sequence
  Primer
gtgtggagtcaccagaccct

SEQ ID NO: 25 - DNA, 20 bp, Artificial Sequence
  Primer
gcttctacttgcagcccatc

SEQ ID NO: 26 - DNA, 24 bp, Artificial Sequence
  Primer
cttaaatgggaggcacagtagaat

SEQ ID NO: 27 - DNA, 24 bp, Artificial Sequence
  Primer
cagtacactttatgcttgggagaa

SEQ ID NO: 28 - DNA, 20 bp, Artificial Sequence
  Primer
gtaacccgcacaccaatttc

SEQ ID NO: 29 - DNA, 20 bp, Artificial Sequence
  Primer
gtgagacatggggatgacct

SEQ ID NO: 30 - DNA, 20 bp, Artificial Sequence
  Primer
cgtctcaggccttcagtgag

SEQ ID NO: 31 - protein, 12 aa, Artificial Sequence
  Synthetically generated peptide
APRRTRVNGDNR